(19) United States
(12) Patent Application Publication
(10) Pub. No.: US 2022/0177947 A1
(43) Pub. Date: Jun. 9, 2022

(54) Compounds to Identify Beta-Lactamases, and Methods of Use Thereof

(71) Applicant: The Regents of the University of California, Oakland, CA (US)

(72) Inventors: Tara Renee DeBoer, Berkeley, CA (US); Nicole Jeanne Tarlton, Berkeley, CA (US); Niren Murthy, Berkeley, CA (US); Lee W. Riley, Berkeley, CA (US); Angel Resendez, Berkeley, CA (US); Nicole Jackson, Berkeley, CA (US)

(73) Assignee: The Regents of the University of California, Oakland, CA (US)

(21) Appl. No.: 17/676,211

(22) Filed: Feb. 20, 2022

Related U.S. Application Data
(63) Continuation of application No. PCT/US20/48060, filed on Aug. 26, 2020.
(60) Provisional application No. 62/893,801, filed on Aug. 29, 2019.

(51) Int. Cl. C12Q 1/34; C07D 501/36; C07D 477/14

(52) U.S. Cl. CPC C12Q 1/34; C12Y 305/02006; C07D 477/14; C07D 501/36

(57) ABSTRACT

Provided herein are β-lactamase probes that can be used to identify specific types and classes of β-lactamases in a sample, and methods of use thereof.

### CROSS REFERENCE TO RELATED APPLICATIONS

This application claims priority under 35 U.S.C. § 119 from Provisional Application Ser. No. 62/893,801, filed Aug. 29, 2020, the disclosure of which is incorporated herein by reference.

### STATEMENT OF GOVERNMENT SUPPORT

This invention was made with government support under Grant Number AI117064 awarded by the National Institutes of Health. The government has certain rights in the invention.

### TECHNICAL FIELD

Provided herein are compounds that can be used to identify specific types and classes of β-lactamases in a sample, and methods of use thereof.

### INCORPORATION BY REFERENCE OF SEQUENCE LISTING

Accompanying this filing is a Sequence Listing entitled “Sequence_ST25.txt”, created on Aug. 26, 2020 and having 4,252 bytes of data, machine formatted on IBM-PC, MS-Windows operating system. The sequence listing is hereby incorporated herein by reference in its entirety for all purposes.

### BACKGROUND

β-lactamases represent an important diagnostic target because they direct resistance to β-lactam antibiotics and their presence in a patient sample can significantly influence clinical decision making. Efforts made for direct or indirect β-lactamase detection by biochemical assays have relied on chromogenic, fluorogenic, or chemiluminescent chemical probes, translation of these approaches to clinical settings have been limited due to poor sensitivity. This sensitivity remains to be an issue which stem from the number of bacteria required to induce conditions of infectious disease are low, ranging from 1 CFU/mL to 10,000 CFU/mL (CFU, colony forming units), detection of the enzymes expressed by these bacteria that confer antibiotic resistance require laborious and time-consuming culturing and/or expensive analytical instrumentation.

Advanced instrumentation such as PCR, matrix assisted laser desorption ionization mass spectrometry, and microscopy have been considered as an approach to enhance detection limits of pathogenic bacteria. However, this strategy is only practical for developed countries and there remains an unmet need of having a reliable diagnostic tool that can be utilized globally, particularly for low- and middle-income (LMIC) countries where resources can be limited.

### SUMMARY

The disclosure provides β-lactamase probes and methods and systems for using these probes in an amplification system to detect activity of β-lactamase variants. Also disclosed are methods of determining β-lactam resistance in a biological sample, the method comprises contacting a sample obtained from a subject with the β-lactamase probe and amplification assay mixture, where the colored or fluorescence product is measured; and correlating the extent of the colored or fluorescence product to β-lactam resistance in a sample that pertain to urinary tract infections. Also disclosed are methods of differentiating between β-lactamase variants that may be present in a biological sample; where the color or fluorescence product that is measured is altered by inhibition of a target β-lactamase by an inhibitor (e.g., include but not limited to clavulanic acid, sulbactam, tazobactam, or RPX7009). Also disclosed are methods for conducting antibiotic susceptibility testing in a biological sample obtained from a subject and contacting said sample with an antibiotic drug, β-lactamase probe, and amplification assay mixture, and measuring the colored or fluorescence product; correlating the extent of the colored or fluorescence product to drug susceptibility wherein a decrease or no optical signal output indicates susceptibility and an increase in signal output indicates resistance to the drug in question.

In a particular embodiment, the disclosure provides for a compound having the structure of Formula I or Formula II:

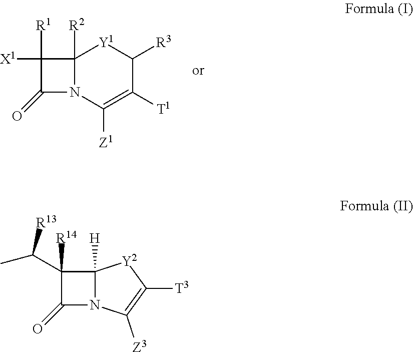

or a salt, stereoisomer, tautomer, polymorph, or solvate thereof, wherein: T¹ is a benzenethiol containing group or Z², wherein if T¹ is Z², then Z¹ is T²; Z¹ is a carboxylate, a carbonyl, an ester, an amide, a sulfone, a sulfonamide, a sulfonyl, —S(O)₂OH or T², wherein if Z¹ is T², then T¹ is Z²; T² is a benzenethiol containing group; T³ is a benzenethiol containing group; Z2 is a carboxylate, a carbonyl, an ester, an amide, a sulfone, a sulfonamide, a sulfonyl, or —S(O)₂OH; Z³ is a carboxylate, a carbonyl, an ester, an amide, a sulfone, a sulfonamide, a sulfonyl, or —S(O)₂OH; X¹ is

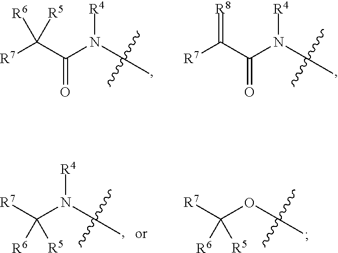

### Y¹ is

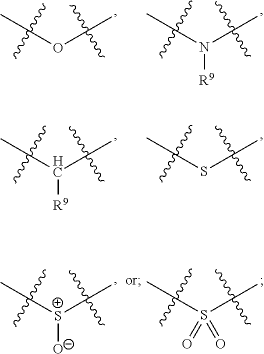

### Y² is

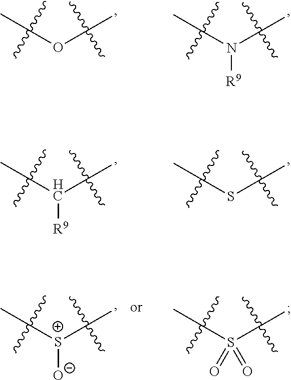

R¹-R⁶, R⁹-R¹¹, R¹³ and R¹⁴ are each independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle; R⁷ is an optionally substituted (C₅-C₇) cycloalkyl, optionally substituted aryl, optionally substituted benzyl, or optionally substituted heterocycle; and R⁸ is

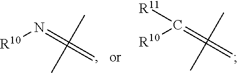

with the proviso that the compound does not have the structure of:

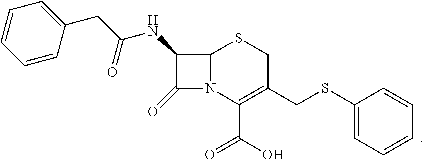 [SMILES: O=C(Cc1ccccc1)N[C@@H]1C(=O)N2C(C(=O)O)=C(CSc3ccccc3)CSC12]

In another embodiment or a further embodiment of any of the foregoing embodiments, T¹ or T² is a benzenethiol group selected from the group consisting of:

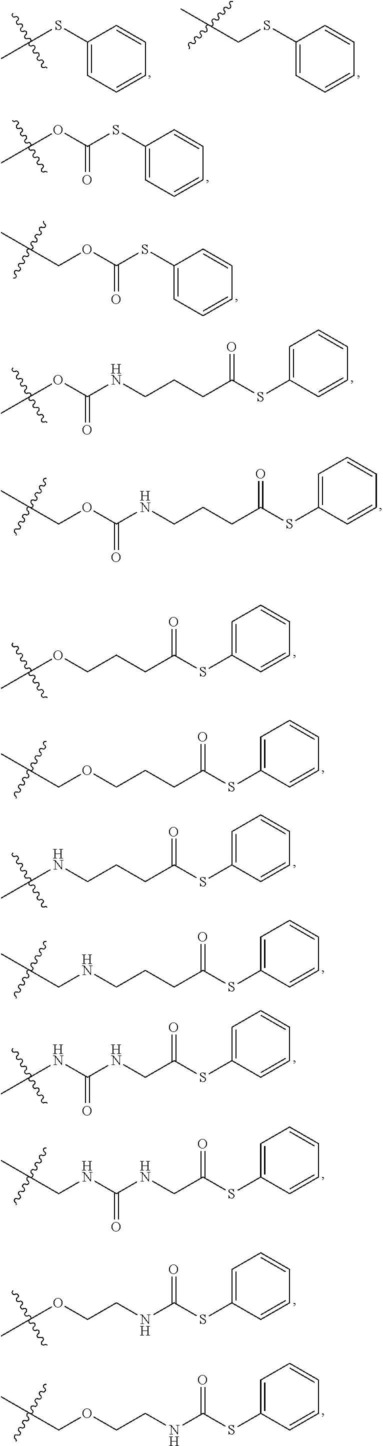

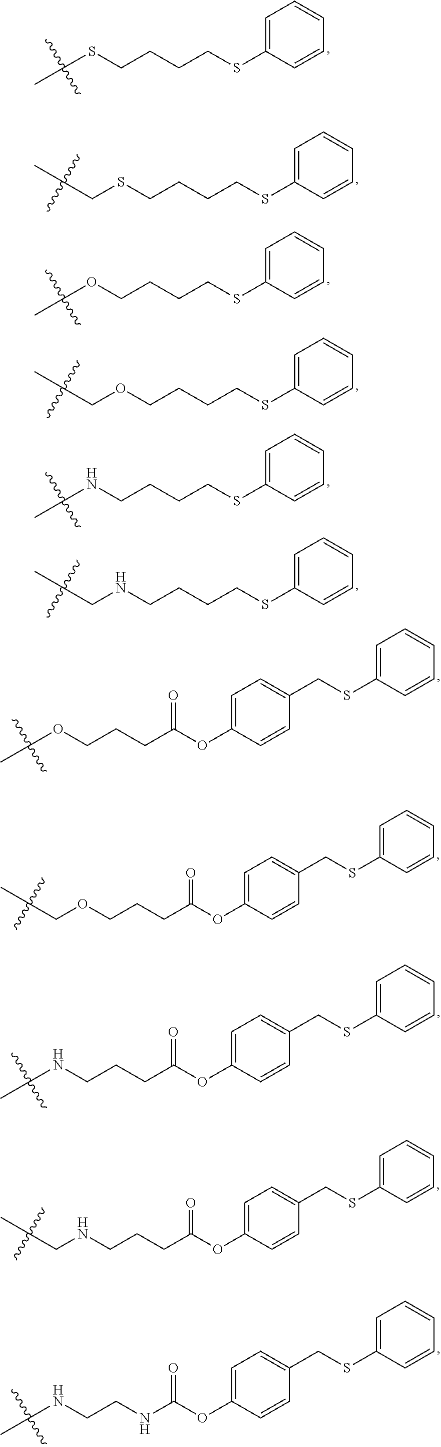

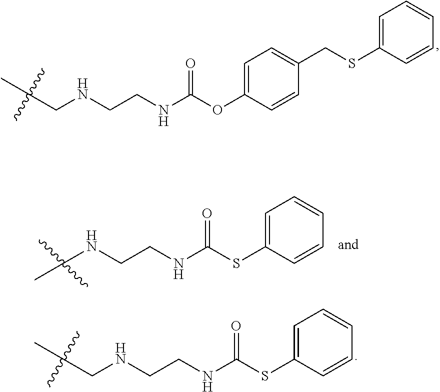 [SMILES: *CNCCNC(=O)Oc1ccc(CSc2ccccc2)cc1 ; *NCCNC(=O)Sc1ccccc1 ; *CNCCNC(=O)Sc1ccccc1]

In another embodiment or a further embodiment of any of the foregoing embodiments, R⁷ is selected from the group consisting of:

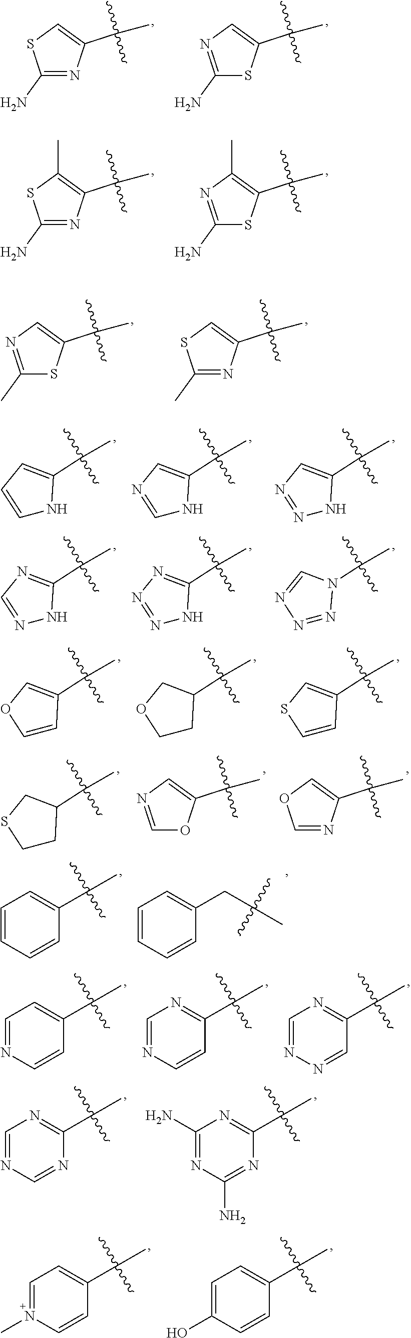

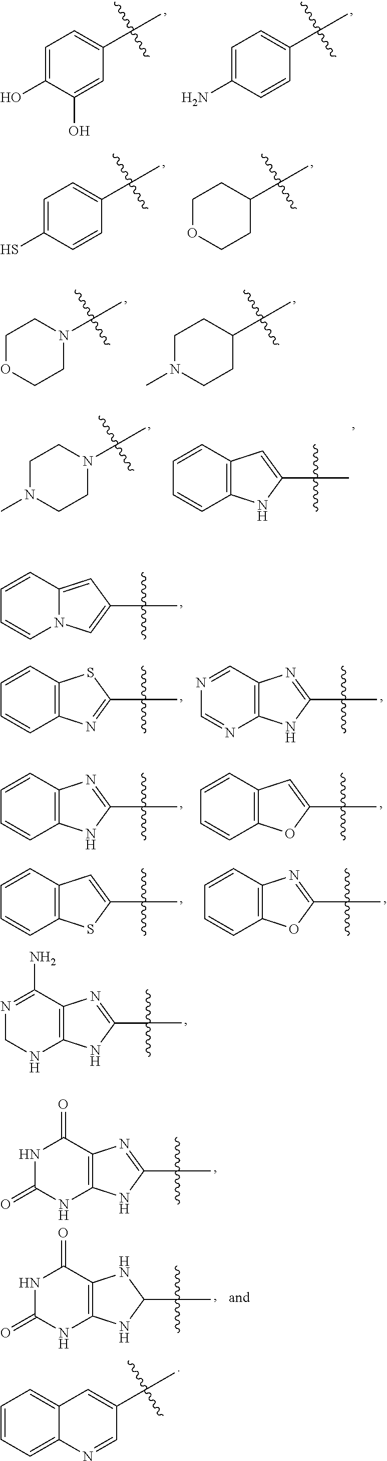

In another embodiment or a further embodiment of any of the foregoing embodiments, the compound has a structure of Formula I(a):

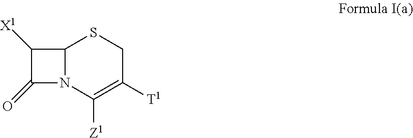

or a salt, stereoisomer, tautomer, polymorph, or solvate thereof, wherein: T¹ is a benzenethiol containing group or Z², wherein if T¹ is Z², then Z¹ is T²; Z¹ is a carboxylate, a carbonyl, an ester, an amide, a sulfone, a sulfonamide, a sulfonyl, —S(O)₂OH or T², wherein if Z is T², then T¹ is Z²; T² is a benzenethiol containing group; Z² is a carboxylate, a carbonyl, an ester, an amide, a sulfone, a sulfonamide, a sulfonyl, or —S(O)₂OH; X¹ is

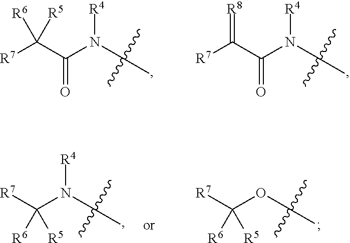

R⁴, R⁵, and R¹⁰ are independently an H or a (C₁-C₆)alkyl; R⁶ is an H, or an amine; R⁷ is an optionally substituted (C₅-C₇) cycloalkyl, optionally substituted aryl, optionally substituted benzyl, or optionally substituted heterocycle; R⁸ is

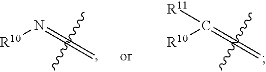

and R⁹ is a hydroxyl or an (C₁-C₃)alkoxy. In another embodiment or a further embodiment of any of the foregoing embodiments, T¹ or T² is a benzenethiol group selected from the group consisting of:

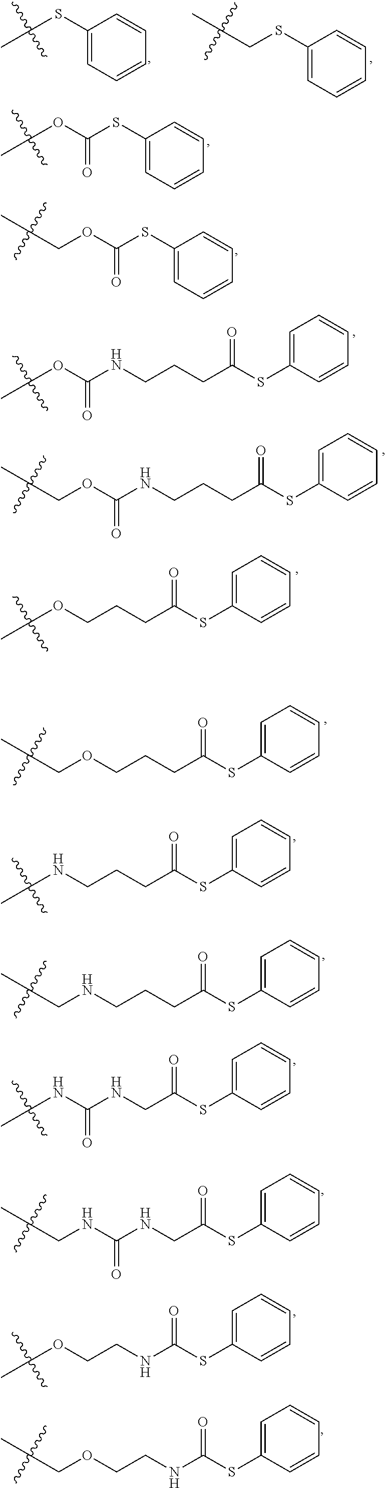

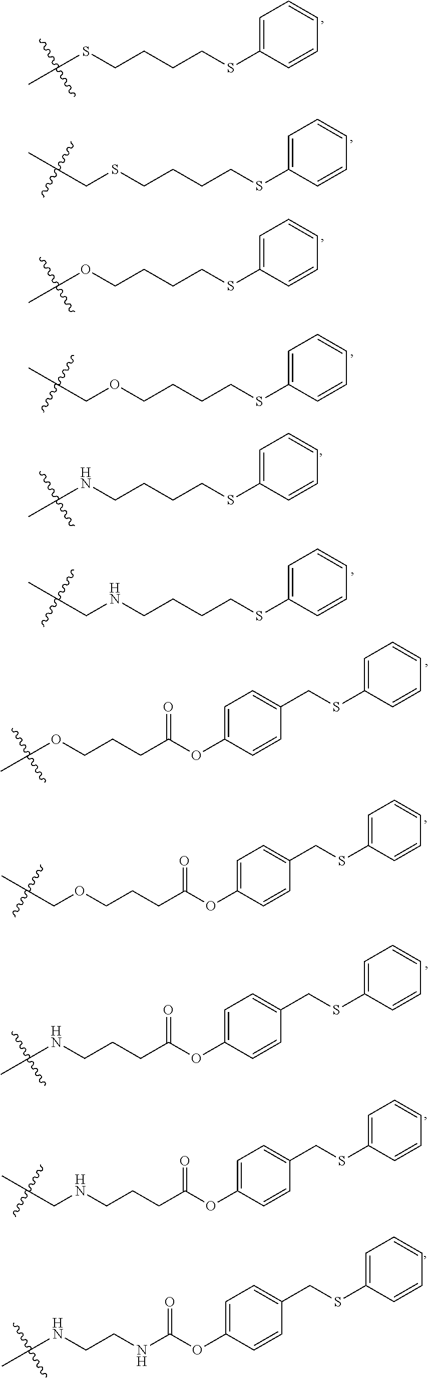

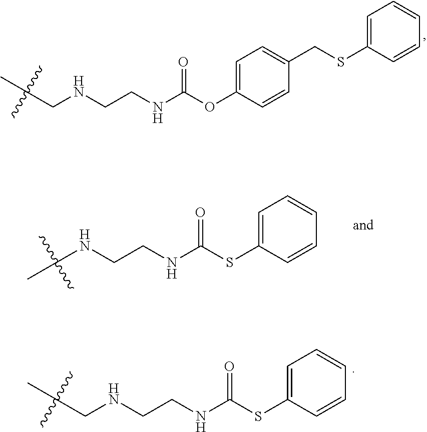 [SMILES: *CNCCNC(=O)Oc1ccc(CSc2ccccc2)cc1 ; *NCCNC(=O)Sc1ccccc1 ; *CNCCNC(=O)Sc1ccccc1]

In another embodiment or a further embodiment of any of the foregoing embodiments, R⁷ is selected from the group consisting of:

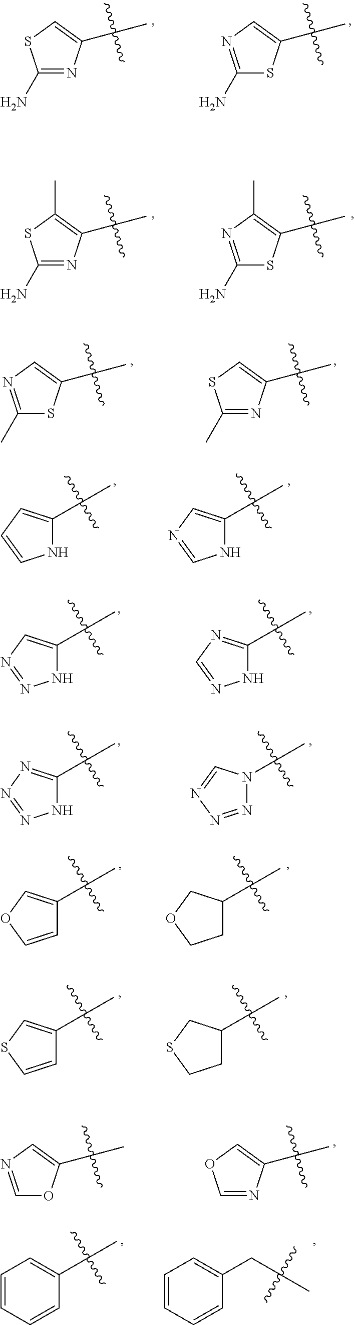

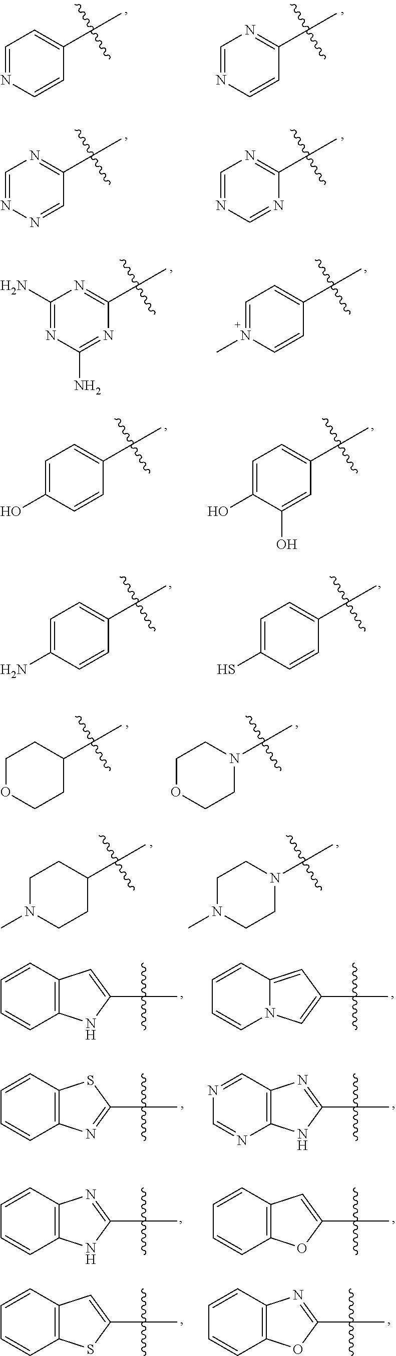

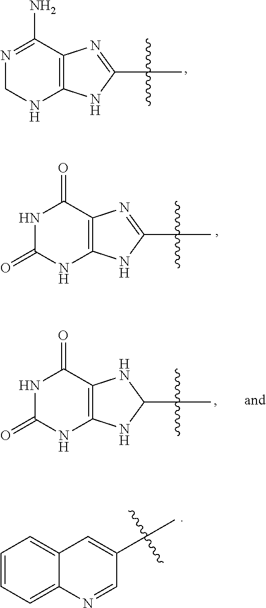 [SMILES: *c1nc2c([nH]1)NCN=C2N ; *c1nc2c(=O)[nH]c(=O)[nH]c2[nH]1 ; *C1Nc2[nH]c(=O)[nH]c(=O)c2N1 ; *c1cnc2ccccc2c1]

In another embodiment or a further embodiment of any of the foregoing embodiments, the compound has the structure of Formula I(b):

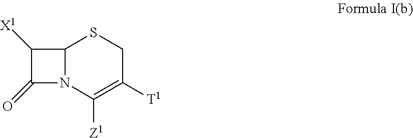

or a salt, stereoisomer, tautomer, polymorph, or solvate thereof, wherein: T¹ a benzenethiol containing group selected from the group consisting of:

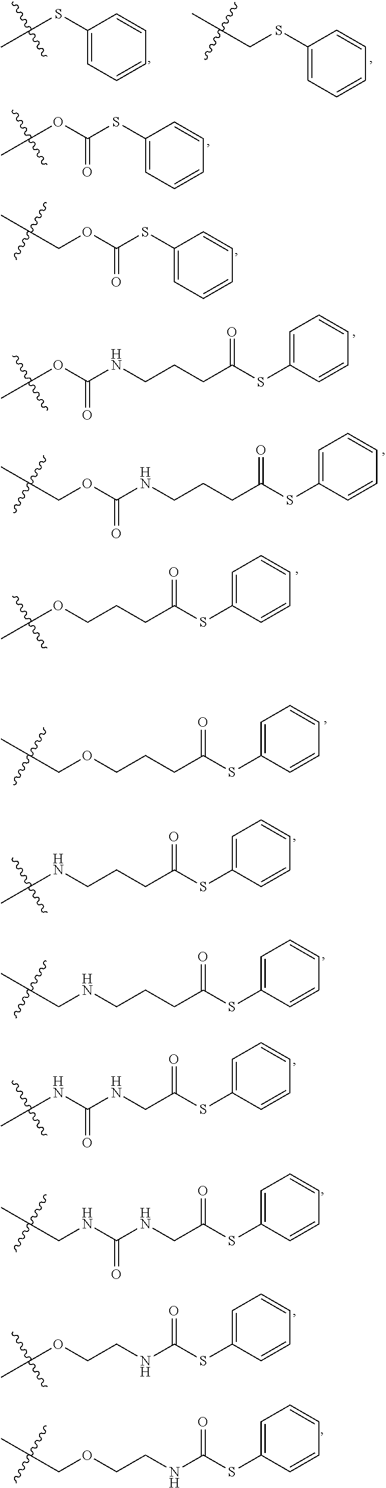

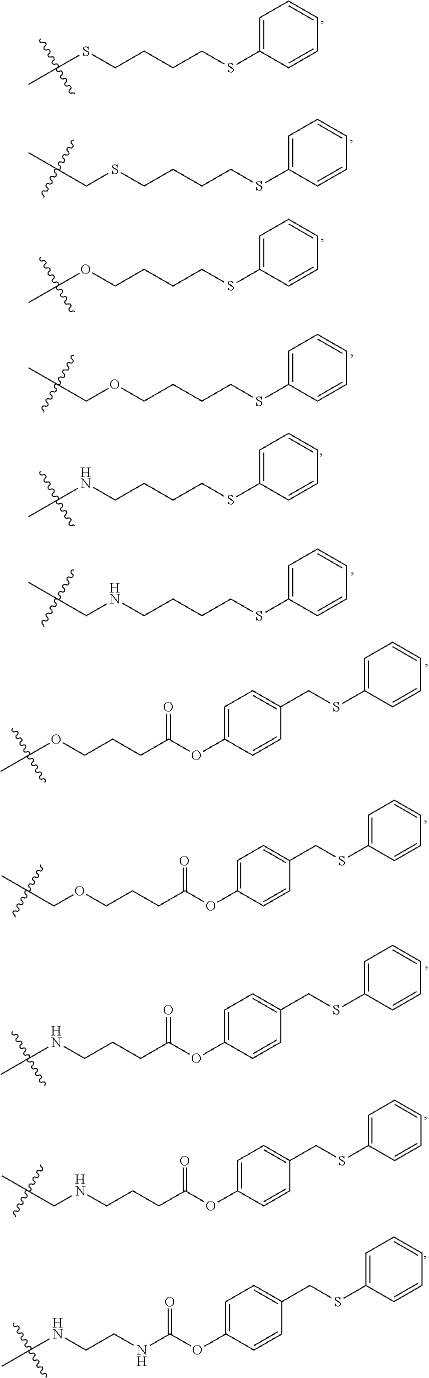

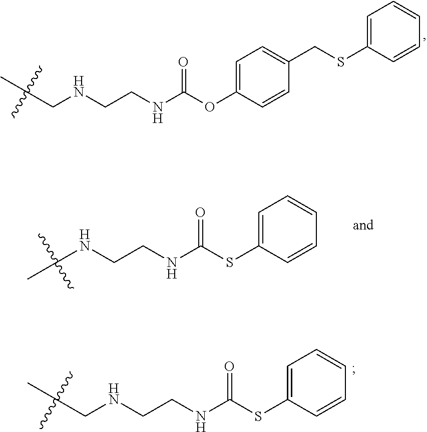 [SMILES: *CNCCNC(=O)Oc1ccc(CSc2ccccc2)cc1 ; *NCCNC(=O)Sc1ccccc1 ; *CNCCNC(=O)Sc1ccccc1]

Z¹ is a carboxylate, a carbonyl, an ester, an amide, a sulfone, a sulfonamide, a sulfonyl, —S(O)₂OH or T²; X¹ is

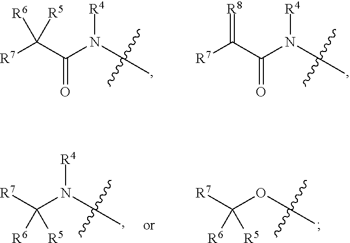

R⁴, R⁵, and R¹⁰ are independently an H or a (C₁-C₆)alkyl; R⁶ is an H, or an amine; R⁷ is an optionally substituted aryl, optionally substituted benzyl, or optionally substituted heterocycle; R⁸ is

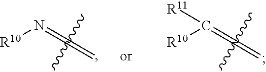

and R⁹ is a hydroxyl or an (C₁-C₃)alkoxy. In another embodiment or a further embodiment of any of the foregoing embodiments, R⁷ is selected from the group consisting of:

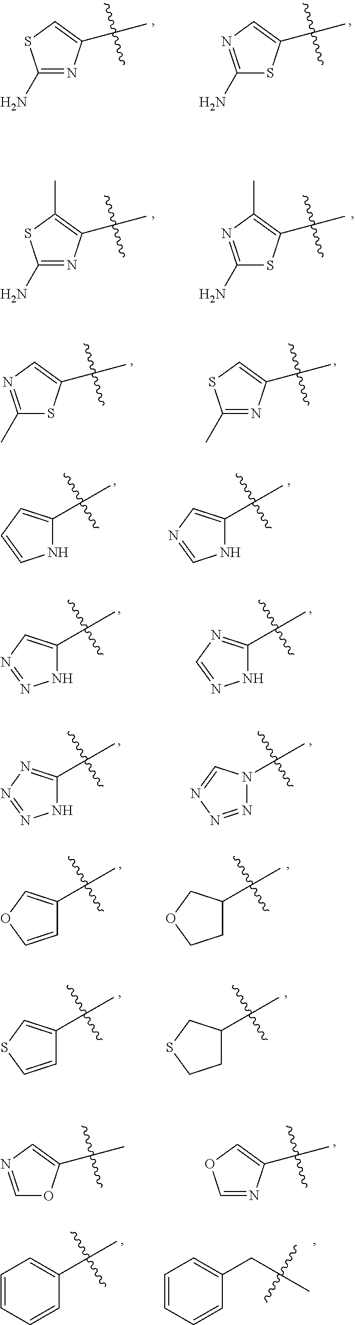

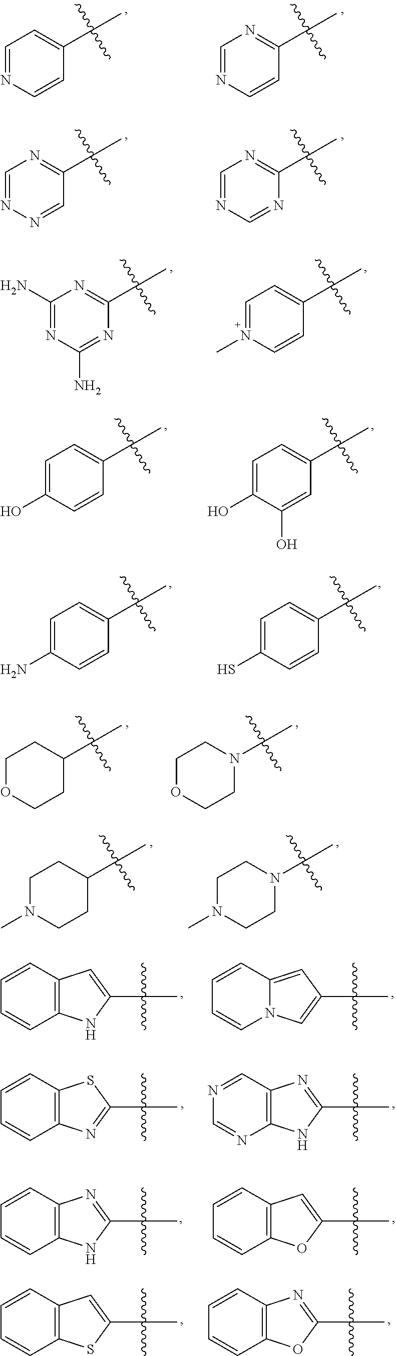

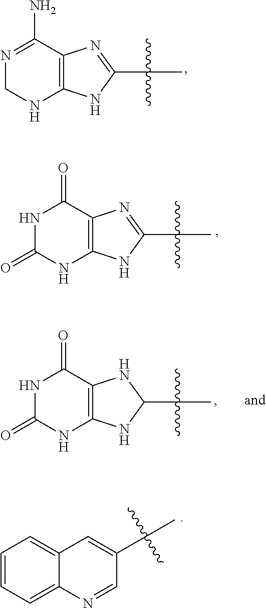 [SMILES: *c1nc2c([nH]1)NCN=C2N ; *c1nc2c(=O)[nH]c(=O)[nH]c2[nH]1 ; *C1Nc2[nH]c(=O)[nH]c(=O)c2N1 ; *c1cnc2ccccc2c1]

In another embodiment or a further embodiment of any of the foregoing embodiments, the compound has the structure of Formula I(c):

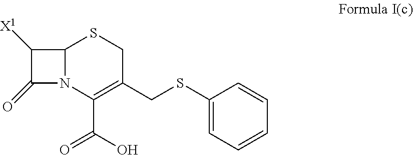

### X¹ is

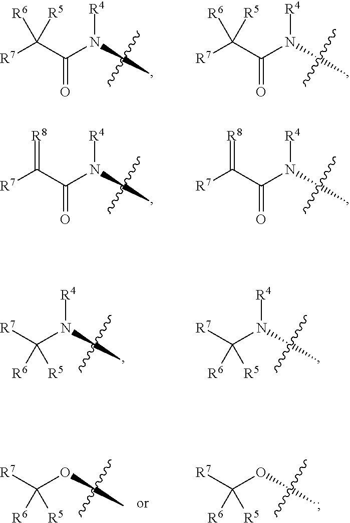

R⁴, R⁵, and R¹⁰ are independently an H or a (C₁-C₆)alkyl; R⁶ is an H, or an amine; R⁷ is selected from the group consisting of:

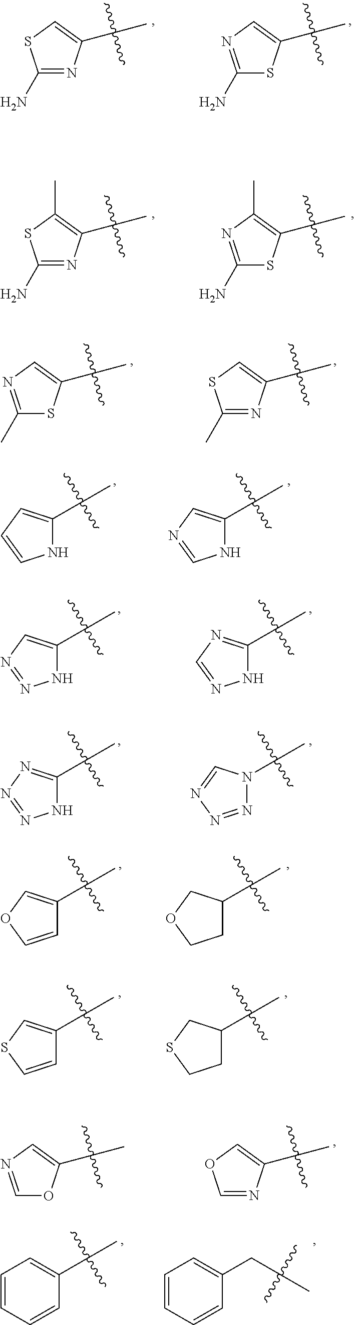

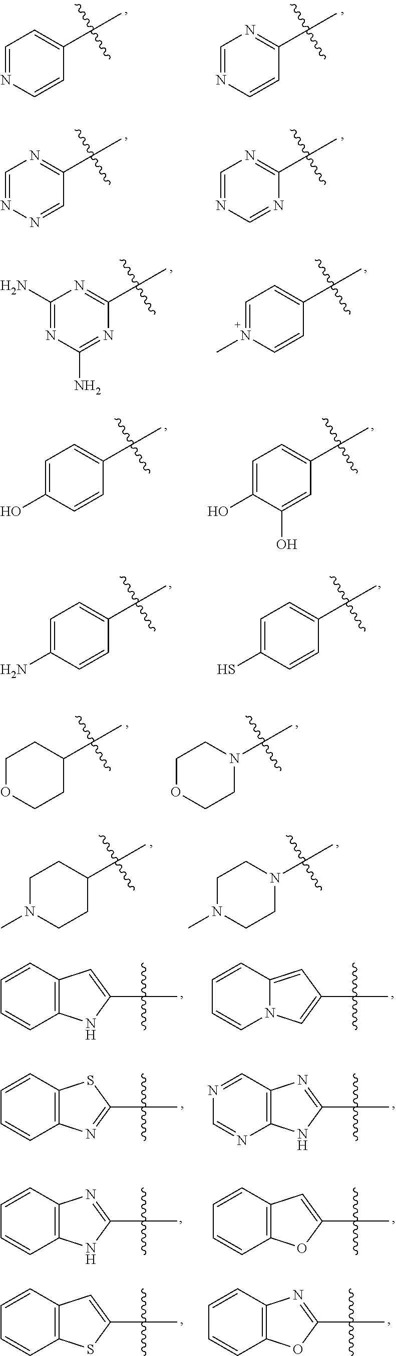

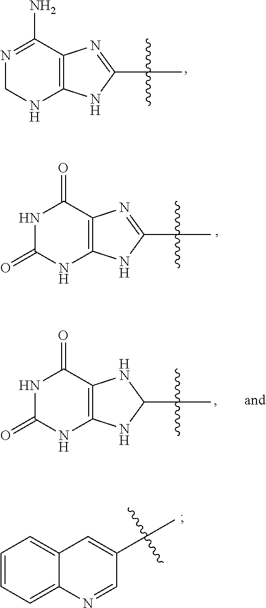 [SMILES: *c1nc2c([nH]1)NCN=C2N ; *c1nc2c(=O)[nH]c(=O)[nH]c2[nH]1 ; *C1Nc2[nH]c(=O)[nH]c(=O)c2N1 ; *c1cnc2ccccc2c1]

### R⁸ is

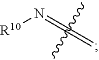

### and R⁹ is

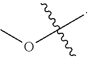

In another embodiment or a further embodiment of any of the foregoing embodiments, the compound is selected from the group consisting of:

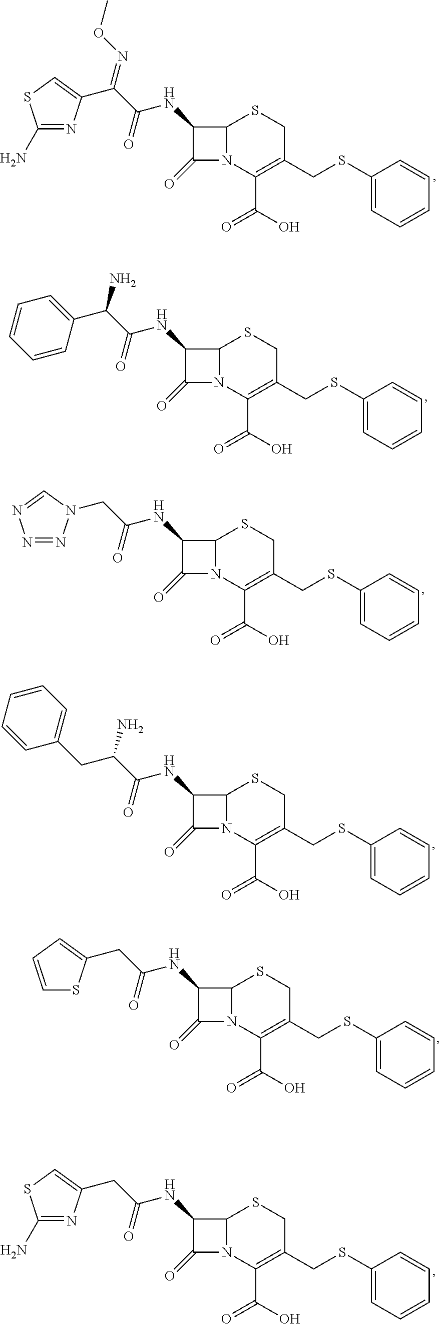 [SMILES: CO/N=C(/C(=O)N[C@@H]1C(=O)N2C(C(=O)O)=C(CSc3ccccc3)CSC12)c1csc(N)n1 ; N[C@@H](C(=O)N[C@@H]1C(=O)N2C(C(=O)O)=C(CSc3ccccc3)CSC12)c1ccccc1 ; O=C(Cn1cnnn1)N[C@@H]1C(=O)N2C(C(=O)O)=C(CSc3ccccc3)CSC12 ; N[C@@H](Cc1ccccc1)C(=O)N[C@@H]1C(=O)N2C(C(=O)O)=C(CSc3ccccc3)CSC12 ; O=C(Cc1cccs1)N[C@@H]1C(=O)N2C(C(=O)O)=C(CSc3ccccc3)CSC12 ; Nc1nc(CC(=O)N[C@@H]2C(=O)N3C(C(=O)O)=C(CSc4ccccc4)CSC23)cs1]

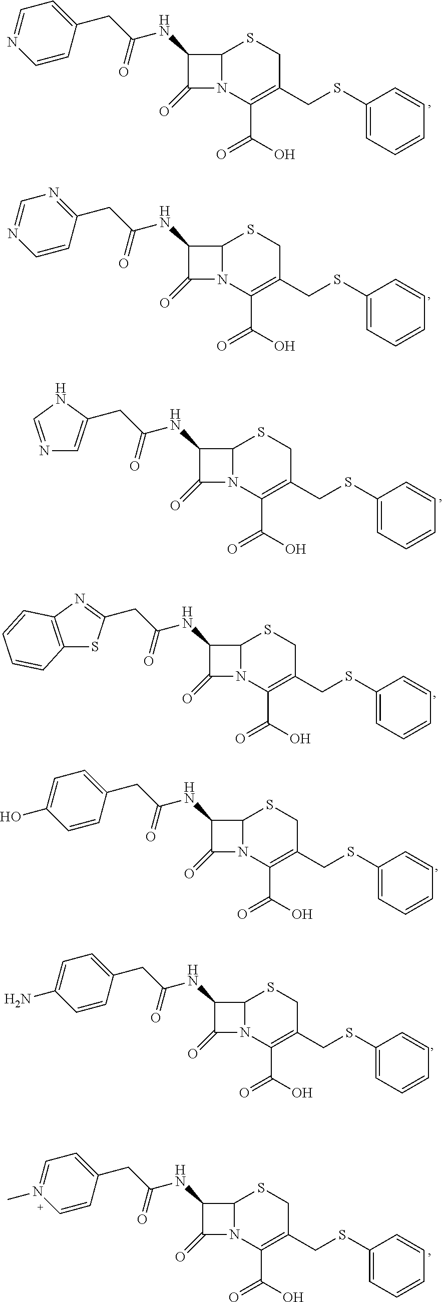

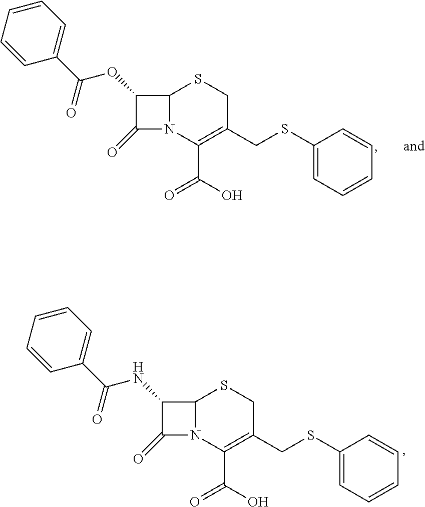 [SMILES: O=C(O)C1=C(CSc2ccccc2)CSC2[C@@H](OC(=O)c3ccccc3)C(=O)N12 ; O=C(O)C1=C(CSc2ccccc2)CSC2[C@@H](NC(=O)c3ccccc3)C(=O)N12]

or a salt, stereoisomer, tautomer, polymorph, or solvate thereof. In another embodiment or a further embodiment of any of the foregoing embodiments, the compound has the structure of:

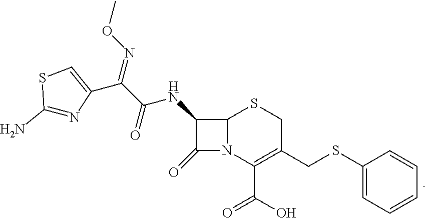 [SMILES: CO/N=C(/C(=O)N[C@@H]1C(=O)N2C(C(=O)O)=C(CSc3ccccc3)CSC12)c1csc(N)n1]

In another embodiment or a further embodiment of any of the foregoing embodiments, T³ is a benzenethiol containing group selected from the group consisting of:

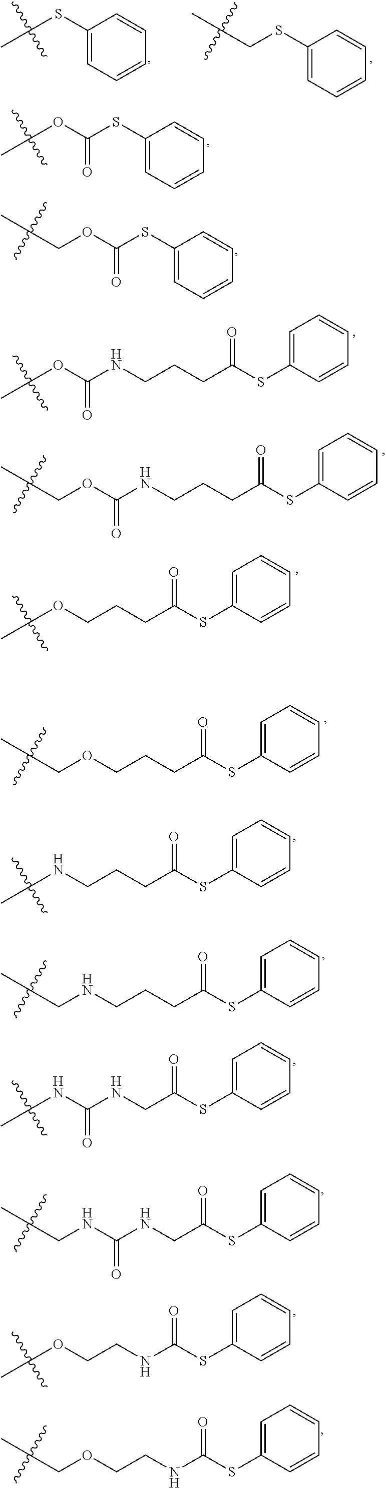

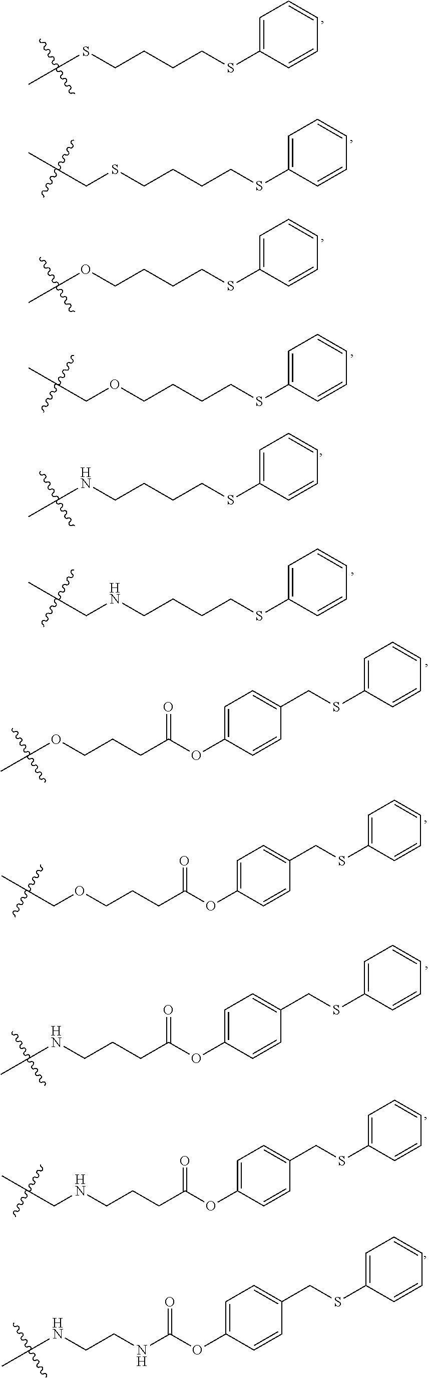

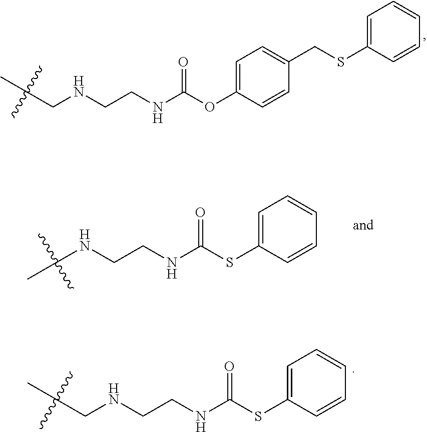 [SMILES: *CNCCNC(=O)Oc1ccc(CSc2ccccc2)cc1 ; *NCCNC(=O)Sc1ccccc1 ; *CNCCNC(=O)Sc1ccccc1]

In another embodiment or a further embodiment of any of the foregoing embodiments, the compound has the structure of Formula II(a):

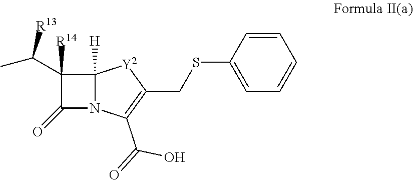

or a salt, stereoisomer, tautomer, polymorph, or solvate thereof, wherein: Y² is

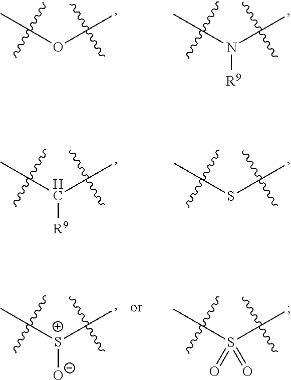

R⁹, R¹³ and R¹⁴ are independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle. In another embodiment or a further embodiment of any of the foregoing embodiments, the compound has the structure of Formula II(b):

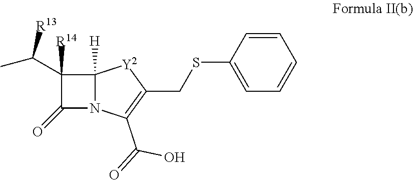

or a salt, stereoisomer, tautomer, polymorph, or solvate thereof, wherein: Y² is

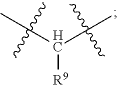

R⁹, R¹³ and R¹⁴ are independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, and optionally substituted (C₁-C₆)alkyl. In another embodiment or a further embodiment of any of the foregoing embodiments, the compound has a structure selected from:

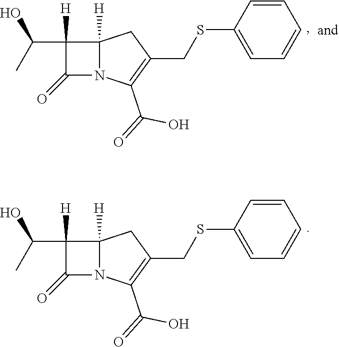 [SMILES: C[C@@H](O)[C@H]1C(=O)N2C(C(=O)O)=C(CSc3ccccc3)C[C@H]12 ; C[C@@H](O)[C@H]1C(=O)N2C(C(=O)O)=C(CSc3ccccc3)C[C@H]12]

In another embodiment or a further embodiment of any of the foregoing embodiments, the compound is substantially a single enantiomer or a single diastereomer, wherein the compound has an (R) stereocenter.

The disclosure also provides a method to detect the presence of one or more target β-lactamases in a sample, comprising: (1) adding reagents to a sample suspected of comprising one or more target β-lactamases, wherein the reagents comprise: (i) a compound of the disclosure; (ii) a chromogenic substrate for a cysteine protease; (iii) a caged/inactive cysteine protease; and (iv) optionally, an inhibitor to specific type(s) or class(es) of β-lactamases; (2) measuring the absorbance of the sample; (3) incubating the sample for at least 10 min and then re-measuring the absorbance of the sample; (4) calculating a score by subtracting the absorbance of the sample measured in step (2) from the absorbance of the sample measured in step (3); (5) comparing the score with an experimentally determined threshold value; wherein if the score exceeds a threshold value indicates that the sample comprises the one or more target β-lactamases; and wherein if the score is lower than the threshold value indicates the sample does not comprise the one or more target β-lactamases. In another embodiment or a further embodiment of any of the foregoing embodiments, for step (1), the sample is obtained from a subject. In another embodiment or a further embodiment of any of the foregoing embodiments, the subject is a human patient that has or is suspected of having a bacterial infection. In another embodiment or a further embodiment of any of the foregoing embodiments, the human patient has or is suspected of having a urinary tract infection. In another embodiment or a further embodiment of any of the foregoing embodiments, for step (1), the sample is a blood sample, a urine sample, a cerebrospinal fluid sample, a saliva sample, a rectal sample, a urethral sample, or an ocular sample. In another embodiment or a further embodiment of any of the foregoing embodiments, for step (1), the sample is a blood sample or urine sample. In another embodiment or a further embodiment of any of the foregoing embodiments, for step (1), the sample is a urine sample. In another embodiment or a further embodiment of any of the foregoing embodiments, for step (1), the one or more target β-lactamases are selected from penicillinases, extended-spectrum β-lactamases (ESBLs), inhibitor-resistant β-lactamases, AmpC-type β-lactamases, and carbapenemases. In another embodiment or a further embodiment of any of the foregoing embodiments, the ESBLs are selected from TEM β-lactamases, SHV β-lactamases, CTX-M β-lactamases, OXA β-lactamases, PER β-lactamases, VEB β-lactamases, GES β-lactamases, and IBC β-lactamase. In another embodiment or a further embodiment of any of the foregoing embodiments, the one or more target β-lactamases comprise CTX-M β-lactamases. In another embodiment or a further embodiment of any of the foregoing embodiments, the carbapenemases are selected from metallo-β-lactamases, KPC β-lactamases, Verona integron-encoded metallo-β-lactamases, oxacillinases, CMY β-lactamases, New Delhi metallo-β-lactamases, Serratia marcescens enzymes, IMIpenem-hydrolysing β-lactamases, NMC β-lactamases and CcrA β-lactamases. In another embodiment or a further embodiment of any of the foregoing embodiments, the one or more target β-lactamases comprise CMY β-lactamases and/or KPC β-lactamases. In another embodiment or a further embodiment of any of the foregoing embodiments, the one or more target β-lactamases further comprise CTX-M β-lactamases. In another embodiment or a further embodiment of any of the foregoing embodiments, for step (1)(ii), the chromogenic substrate for a cysteine protease is a chromogenic substrate for papain, bromelain, cathepsin K, calpain, caspase-1, galactosidase, seperase, adenain, pyroglutamyl-peptidase I, sortase A, hepatitis C virus peptidase, sindbis virus-type nsP2 peptidase, dipeptidyl-peptidase VI, deSI-1 peptidase, TEV protease, amidophosphoribosyl transferase precursor, gamma-glutamyl hydrolase, hedgehog protein, or dmpA aminopeptidase. In another embodiment or a further embodiment of any of the foregoing embodiments, the chromogenic substrate for a cysteine protease is a chromogenic substrate for papain. In another embodiment or a further embodiment of any of the foregoing embodiments, the chromogenic substrate for papain is selected from the group consisting of azocasein, L-pyroglutamyl-L-phenylalanyl-L-leucine-p-nitroanilide (PFLNA), Nα-benzoyl-L-arginine 4-nitroanilide hydrochloride (BAPA), pyroglutamyl-L-phenylalanyl-L-leucine-p-nitroanilide (Pyr-Phe-Leu-pNA), and Z-Phe-Arg-p-nitroanilide. In another embodiment or a further embodiment of any of the foregoing embodiments, the chromogenic substrate for papain is BAPA. In another embodiment or a further embodiment of any of the foregoing embodiments, for step (1)(iii), the caged/inactive cysteine protease comprises a cysteine protease selected from the group consisting of papain, bromelain, cathepsin K, calpain, caspase-1, galactosidase, seperase, adenain, pyroglutamyl-peptidase I, sortase A, hepatitis C virus peptidase, sindbis virus-type nsP2 peptidase, dipeptidyl-peptidase VI, deSI-1 peptidase, TEV protease, amidophosphoribosyl transferase precursor, gamma-glutamyl hydrolase, hedgehog protein, and dmpA aminopeptidase. In another embodiment or a further embodiment of any of the foregoing embodiments, the caged/inactive cysteine protease comprises papain. In another embodiment or a further embodiment of any of the foregoing embodiments, the caged/inactive cysteine protease is papapin-S—SCH₃. In another embodiment or a further embodiment of any of the foregoing embodiments, for step (1)(iii), the caged/inactive cysteine protease can be re-activated by reaction with low molecular weight thiolate anions or inorganic sulfides. In another embodiment or a further embodiment of any of the foregoing embodiments, the caged/inactive cysteine protease can be reactivated by reaction with a benzenethiolate anion. In another embodiment or a further embodiment of any of the foregoing embodiments, the one or more target β-lactamases react with the compound of (i) to produce a benzenethiolate anion. In another embodiment or a further embodiment of any of the foregoing embodiments, the benzenethiolate anion liberated from the compound of step (1)(i) reacts with the caged/inactive cysteine protease to reactivate the cysteine protease. In another embodiment or a further embodiment of any of the foregoing embodiments, the caged/inactive cysteine protease is papain-S—SCH₃. In another embodiment or a further embodiment of any of the foregoing embodiments, the chromogenic substrate for a cysteine protease is BAPA. In another embodiment or a further embodiment of any of the foregoing embodiments, for step (2), the absorbance of the sample is measured at 0 min. In another embodiment or a further embodiment of any of the foregoing embodiments, for step (3), the sample is incubated for 15 min to 60 min. In another embodiment or a further embodiment of any of the foregoing embodiments, the sample is incubated for 30 min. In another embodiment or a further embodiment of any of the foregoing embodiments, for steps (2) and (3), the absorbance of the sample is measured at a wavelength of 400 nm to 450 nm. In another embodiment or a further embodiment of any of the foregoing embodiments, for steps (2) and (3), the absorbance of the sample is measured at a wavelength of 405 nm. In another embodiment or a further embodiment of any of the foregoing embodiments, for steps (2) and (3), the absorbance of the sample is measured using a spectrophotometer, or a plate reader. In another embodiment or a further embodiment of any of the foregoing embodiments, for step (5), the experimentally determined threshold value was determined by analysis of a receiver operating characteristic (ROC) curve generated from an isolate panel of bacteria that produce β-lactamases, wherein the one of more target β-lactamases have the lowest limit of detection (LOD) in the isolate panel. In another embodiment or a further embodiment of any of the foregoing embodiments, the method is performed with and without the inhibitor to specific type(s) or class(es) of β-lactamase in step (lxiv). In another embodiment or a further embodiment of any of the foregoing embodiments, a measured change in the score of step (4), between the method performed without the inhibitor and the method performed with the inhibitor indicates that the specific type or class of β-lactamases is present in the sample. In another embodiment or a further embodiment of any of the foregoing embodiments, the inhibitor to specific type(s) or class(es) of β-lactamases is an inhibitor to class of β-lactamases selected from the group consisting of penicillinases, extended-spectrum β-lactamases (ESBLs), inhibitor-resistant β-lactamases, AmpC-type β-lactamases, and carbapenemases. In another embodiment or a further embodiment of any of the foregoing embodiments, the inhibitor to a specific type(s) or class(es) of β-lactamases inhibits ESBLs but does not inhibit AmpC-type β-lactamases. In another embodiment or a further embodiment of any of the foregoing embodiments, the inhibitor is clavulanic acid or sulbactam.

Additional enumerated aspects and embodiments of the invention include:

1. A method of using a trigger-releasing chemophore to detect resistant markers, comprising: (a) incubating a clinical sample comprising an extended-spectrum ?-lactamase (ESBL) with a promiscuous cephalosporin chemophore that is hydrolyzed by the lactamase to liberate a thiol trigger; (b) incubating the thiol trigger with a disulfide inactivated amplification enzyme to activate the amplification enzyme in an interchange reaction of the thiol and the disulfide; (c) incubating the activated amplification enzyme with an amplification enzyme substrate to generate an amplified signal; and (d) detecting the amplified signal as an indicator of an Extended-spectrum ?-lactamase (ESBL)-producing bacteria in the sample.

2. The method of aspect 1 wherein the amplification enzyme is a cysteine protease or a protease having cysteine protease activity.

3. The method of aspect 1 wherein the amplification enzyme is a cysteine protease selected from papain, bromelain, cathepsin K, and calpain, caspase-1 and separase, adenain, pyroglutamyl-peptidase I, sortase A, hepatitis C virus peptidase 2, sindbis virus-type nsP2 peptidase, dipeptidyl-peptidase VI, deSI-1 peptidase, TEV protease, amidophosphoribosyltransferase precursor, gamma-glutamyl hydrolase, hedgehog protein, and dmpA aminopeptidase.

4. The method of aspect 1 wherein the chemophore comprises a sulfenyl moiety, that is cleaved by the target enzyme to liberate a corresponding aromatic or alkyl thiol via an elimination mechanism.

5. The method of aspect 1 wherein the chemophore is a structure disclosed herein.

6. The method of aspect 1 wherein the amplification enzyme substrate generates a colored or fluorescent product.

7. The method of aspect 1 wherein the amplification enzyme substrate generates an autocatalytic secondary amplifier.

8. The method of aspect 1 wherein the amplification enzyme substrate generates an autocatalytic secondary amplifier, that is a peptide, which liberates a self-immolative chemical moiety upon hydrolytic cleavage of the backbone peptide, to undergo intramolecular cyclization or elimination mechanisms and evolve additional thiol species to trigger further cysteine protease molecules.

9. The method of aspect 1 wherein the amplification enzyme is papain, and the amplification enzyme substrate is a papain probe having a structure disclosed herein.

10. The method of aspect 1 wherein the amplification enzyme is papain, and the amplification enzyme substrate is a papain probe having a structure disclosed herein and the thiol-releasing chemophore has a structure disclosed herein.

11. The method of aspect 1 wherein the sample is unprocessed urine.

12. The method of aspect 1 wherein the sample is a patient sample, and the method further comprises treating the patient for an infection caused by a bacterial pathogen resistant to a ?-lactam antibiotic.

13. The method of aspect 1 wherein the sample is a patient unprocessed urine sample, and the method further comprises treating the patient for an urinary tract infection (UTI) of a bacterial pathogen resistant to a ?-lactam antibiotic.

The invention encompasses all combinations of the particular embodiments recited herein, as if each combination had been laboriously recited.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1 provides an overview of an embodiment of a DETECT assay that can be applied to reveal CTX-M β-lactamase activity directly in clinical urine samples. A representation of the experimental workflow applied to analyze a urine sample by DETECT. A small volume of urine is transferred into a well containing DETECT reagents (D; steps 1 and 2). The absorbance at 405 nm (A_{405 nm}) is recorded with a spectrophotometer at 0 min. If the target resistance marker is present (E1; a CTX-M ESBL enzyme) the targeting probe is hydrolyzed and the thiophenol trigger eliminates from the probe, subsequently activating the amplification and colorimetric signal output tier of DETECT (step 3). After 30 min of room temperature incubation an A_{405 nm} reading is again recorded, and the DETECT score is calculated (step 4; A_{405 nm} T30-T0). A DETECT score exceeding an experimentally determined threshold value indicates the sample contains the target CTX-M β-lactamase, and hence, an expanded-spectrum cephalosporin-resistant GNB is present in the urine sample (step 5). A DETECT score that is lower than the threshold value indicates the sample does not contain the target resistance marker. BAPA: Nα-Benzoyl-L-arginine 4-nitroanilide hydrochloride.

FIGS. 2A-2E demonstrates that the DETECT system is preferentially activated by CTX-M and CMY β-lactamases. (A) DETECT's LOD (in nM) at 20 min across diverse recombinant β-lactamases, where a lower bar and lower LOD indicates greater reactivity with the DETECT system. The OXA-1 LOD (not displayed) is >4 μM. (B) Average DETECT score at 30 min from clinical isolates of E. coli and K. pneumoniae. Isolates are grouped based on β-lactamase content in the cells, using the following placement scheme: CTX-M >CMY >KPC >ESBL SHV or ESBL TEM >TEM >SHV or OXA >β-lactam-susceptible. Numbers in square brackets [#] represent number of isolates in each group. Error bars represent standard deviation. Data were analyzed by two-tailed 1-test. P values for each group under the black or blue line were the same for each comparison, so only one P value is listed; **P<0.01, ****P<0.0001. The dotted green line represents the DETECT threshold value generated from ROC curve analyses (0.2806). (C) Expression of bla genes in isolates containing different β-lactamases. Fold-expression of bla genes was determined in comparison to the internal control rpoB, to assess β-lactamase expression across enzymes and isolates. Error bars represent the standard deviation from two biological replicates. Fold-expression of blaKPC-2 exceeds the bounds of the chart, so fold-expression and standard deviation are written in. The right axis illustrates DETECT Score; red-orange circles represent corresponding DETECT Score for each isolate. (D) Comparison of the times-change in DETECT Score at 30 min (DETECT Score divided by DETECT+inhibitor Score) in isolates with CMY or a CTX-M, when the β-lactamase inhibitor clavulanic acid is incorporated into the system. β-lactamase content of the E. coli and K. pneumoniae clinical isolates is indicated on the left axis. The dotted black line represents the positive threshold that is indicative of the presence of CTX-Ms (times-change >1.97×), calculated based on the average times-change in DETECT Score plus three-times its standard deviation in isolates that contain CMY (indicated by yellow bars). (E) Comparison of the average times-change in DETECT score at 30 min in isolates producing CMY or CTX-M, when the β-lactamase inhibitor clavulanic acid is incorporated into the system (times-change=DETECT score/DETECT+inhibitor score). The dotted green line represents the positive threshold that is indicative of the activity of CTX-Ms (times-change >1.97). ****P<0.0001.

FIG. 3 presents a schematic of a urine study workflow, demonstrating standard urine sample testing and testing with DETECT. Urine samples submitted to the clinical laboratory for standard urine culture (i.e., from patients with suspected UTI) were utilized in this study. (A) The top panel represents standard procedures performed by the clinical laboratory for workup of urine samples. Urine samples yielding significant colony counts (≥10⁴ CFU/mL cutoff applied) were further tested by the clinical laboratory. ID, identification; AST, antimicrobial susceptibility testing. (B) The middle panel depicts the microbiology and molecular biology procedures performed by study investigators, which were confirmed by comparison to the clinical laboratory's results (CFU/mL estimates), or guided by the clinical laboratory's ID and AST results. (C) The lower panel illustrates the DETECT testing workflow performed by study investigators. Colorimetric signal (A_{405 nm}) was recorded by a microplate reader.

FIG. 4 presents the profile of clinical urine samples tested with DETECT. (A) Breakdown of organisms causing UTI. While it is assumed that the majority of urine samples submitted to the clinical laboratory for urine culture were submitted from patients with symptoms suggestive of UTI, here “true” UTI was defined by colony counts >10⁴ CFU/mL, a standard microbiological cutoff indicative of UTI. Numbers in square brackets [#] represent number of UTIs caused by the indicated organism group. (B) Breakdown of significant GNB and GPB identified from urine samples. One-hundred and nine GNB were identified from 96 GNB UTIs. Numbers in square brackets [#] represent number of times a bacterial species was identified. (C) Pie chart demonstrating the proportion of ESBL UTIs identified in the total UTI population. (D) Distribution of ESBL-producing GNB and ESBL classes identified in ESBL-positive samples.

FIGS. 5A-5B demonstrates that the DETECT assay identifies UTIs caused by CTX-M-producing bacteria directly from unprocessed urine samples in 30 minutes. (A) Average DETECT score at 30 min from urine samples containing different types of bacteria. Groups include: urine samples that did not grow bacteria (no growth); urine samples that grew bacteria that were not indicative of UTI (no UTI); urine samples from UTIs caused by GPB or yeast (Gram-pos or Yeast UTI); and urine samples from UTIs caused by GNB that contained no β-lactamase detected (no β-lac detected), GNB with SHV (SHV), GNB with TEM (TEM), GNB with an SHV ESBL (SHV ESBL), GNB with a chromosomal AmpC (cAmpC), or GNB with a CTX-M (CTX-M). For group placement of GNB samples when more than one β-lactamase was identified: CTX-M >cAmpC >ESBL SHV or ESBL TEM >TEM >SHV >no β-lactamase detected. The chromosomal AmpC of E. coli was not considered, nor was the chromosomal β-lactamase of K. pneumoniae (unless it was SHV, or LEN variants identified with SHV primers). Thirty-one (89%) “no β-lactamase detected” samples yielded isolates that were susceptible to β-lactams. Numbers in square brackets [#] represent number of samples in each group. Error bars represent the standard deviation. Data were analyzed by two-tailed t-test. P values for each group under the black or blue line were the same for each comparison, so only one P value is listed; *P<0.05, **P<0.01, ***P<0.001. The dotted green line represents the threshold generated from ROC curve analysis (0.2588). (B) DETECT assay specifications for the ability to identify UTIs caused by CTX-M-producing third-generation cephalosporin-resistant GNB. The standard for comparison to DETECT included a phenotypic method for ESBLs (ESBL confirmatory testing) and a genotypic method (PCR with amplicon sequencing for CTX-M genes).

FIGS. 6A-6B shows that CTX-M-producing bacteria are associated with multidrug-resistance (MDR). (A) Antimicrobial resistance phenotypes of Enterobacterales cultured from UTI-positive urine samples, grouped based on CTX-M content. ^{♦}Intrinsic cefoxitin resistance was not included (E. aerogenes, E. hormaechei, C. freundii, and P. agglomerans). ^{⋄}Intrinsic nitrofurantoin and tigecycline resistance was not included (P. mirabilis and P. rettgeri). Data were analyzed by Fisher's exact test. The P value is for the comparison of resistance in CTX-M-producing isolates vs. isolates lacking CTX-Ms; **P<0.01, ***P<0.001, ****P<0.0001. (B) Distribution of multidrug resistance (MDR) in CTX-M-producing bacteria vs. bacteria that do not produce CTX-Ms.

FIGS. 7A-7B details urine sample appearance and pH. (A) Visual appearance of urine samples tested by DETECT, including clarity (turbidity) and color. (B) Urine pH, measured with pH strips. 471 samples are represented in both figures, since one sample did not have its appearance or pH recorded.

FIG. 8 illustrates an overview of the DETECT two-tiered amplification platform technology. DETECT amplification is initiated by a β-lactamase enzyme (e.g., CTXM-14 variant) that hydrolyses the β-lactam analogue substrate and releases the thiol containing trigger unit (T1). The released T1 activates the disulfide-protected papain via a disulfide interchange reaction, producing activated papain (Enzyme Amplifier II). A colorimetric signal is produced by hydrolysis of a peptidyl-indicator (BAPA, E2 substrate) by the activated papain. Analysis of a panel of β-lactamase variants with the DETECT platform provided a specific correlation between the presence of a β-lactamase variant CTXM-14. The β-lactamase probe that was utilized was highly specific for this variant and provided improved detection limits (10⁴ CFU/mL) compared to standard analysis (107 CFU/mL). The colorimetric output signal (the change in the 405 nm absorbance from time 0 to 1 h) resulted in a DETECT score where the threshold value is 3× standard deviation greater than the average DETECT score of control.

FIG. 9 illustrates the detection limits (1/LOD) threshold of the DETECT platform across a panel of purified recombinant β-lactamases (TEM-1, SHV-12, CTXM-14, SHV-1, TEM-20, CMY-2, and KPC-1) tested with each probe.

FIG. 10 illustrates the DETECT score (A of 405 nm absorbance from time 0 to 1 h) of AmpC producing clinical isolates using a β-lactamase probe in combination or absence of a β-lactamase inhibitor such as clavulanic acid and tazobactam.

### DETAILED DESCRIPTION

As used herein and in the appended claims, the singular forms “a,” “an,” and “the” include plural referents unless the context clearly dictates otherwise. Thus, for example, reference to “a β-lactamase substrate” includes a plurality of such substrates and reference to “the β-lactamase” includes reference to one or more-lactamases and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, for terms expressly defined in this disclosure, the definition of the term as expressly provided in this disclosure will control in all respects, even if the term has been given a different meaning in a publication, dictionary, treatise, and the like.

The term “a benzenethiol containing group” as used herein, refers to a group designated herein (e.g., T¹ or T² substituent) that comprises a terminal benzenethiol group which has the structure of:

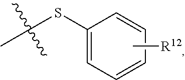

wherein R¹² is H, D, alkoxy, hydroxyl, ester, amide, aryl, heteroaryl, nitro, cyanate, nitrile, or halo. The terminal benzenethiol group of “a benezenethiol containing group” may be directly attached to a compound having a structure designated by Formulas presented herein. Alternatively, the terminal benzenethiol group of “a benezenethiol containing group” may be indirectly attached to a compound having a structure of Formulas I-III by a linker. The linker is either a (C₁-C₁₂)alkyl or a (C₁-C₁₂)heteroalkyl. Examples of “a benezenethiol containing group” for the purposes of this disclosure include, but are not limited to:

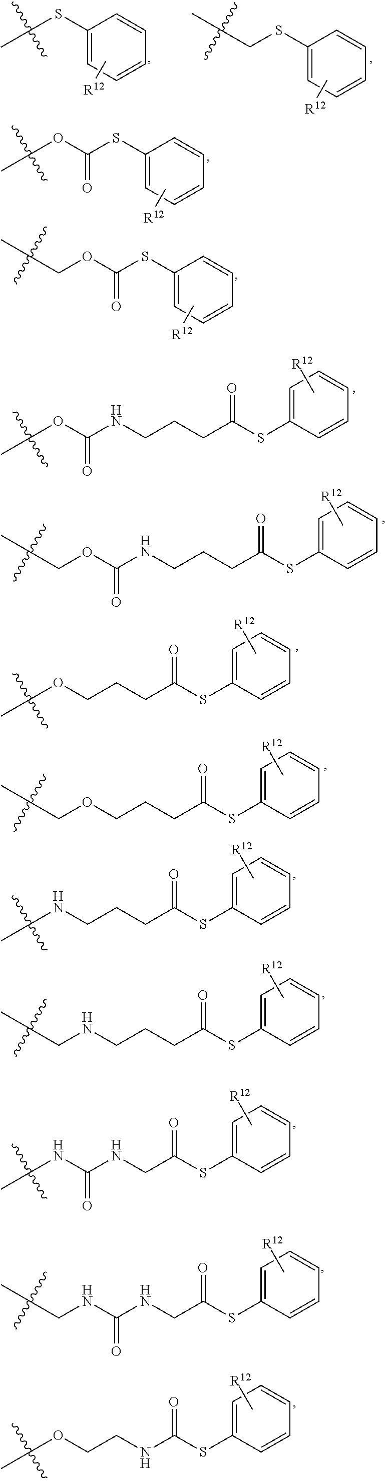

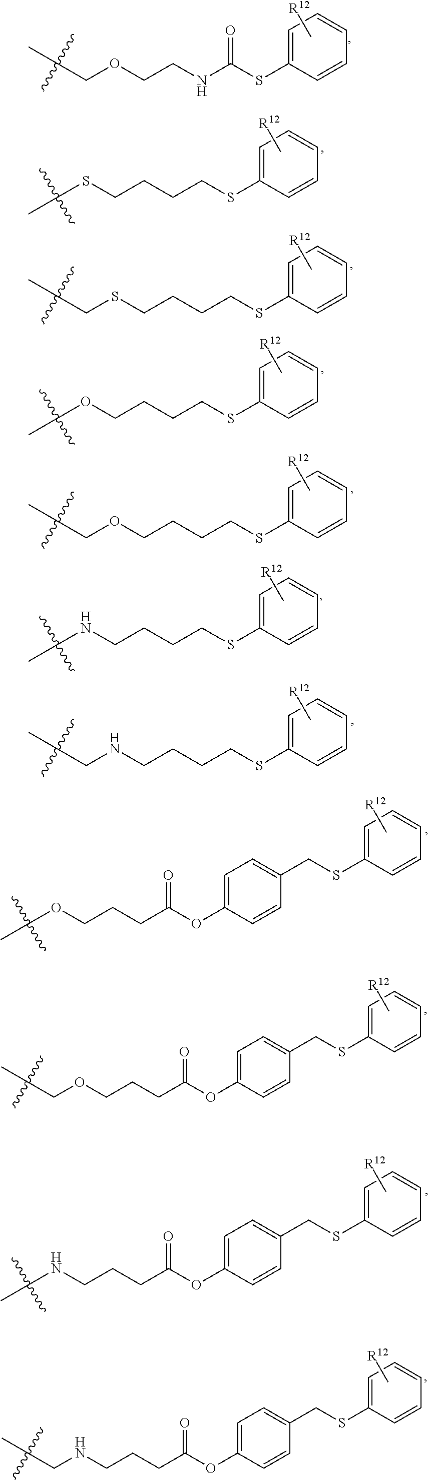

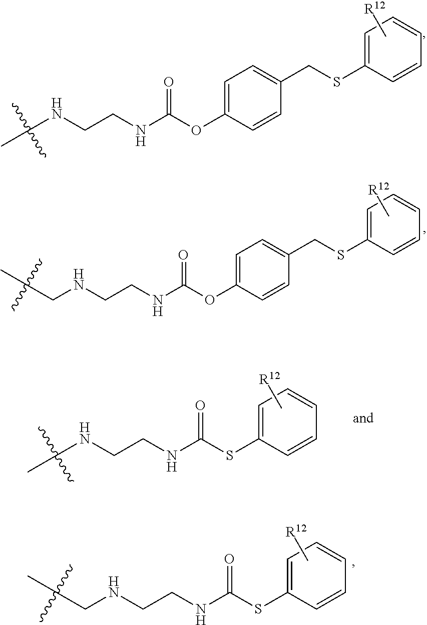

wherein R¹² is H, D, alkoxy, hydroxyl, ester, amide, aryl, heteroaryl, nitro, cyanate, nitrile, or halo. In a particular embodiment, R¹² is H.

The term “hetero-” when used as a prefix, such as, hetero-alkyl, hetero-alkenyl, hetero-alkynyl, or hetero-hydrocarbon, for the purpose of this disclosure refers to the specified hydrocarbon having one or more carbon atoms replaced by non-carbon atoms as part of the parent chain. Examples of such non-carbon atoms include, but are not limited to, N, O, S, Si, Al, B, and P. If there is more than one non-carbon atom in the hetero-based parent chain then this atom may be the same element or may be a combination of different elements, such as N and O. In a particular embodiment, a “heteroalkyl” comprises one or more copies of the following groups,

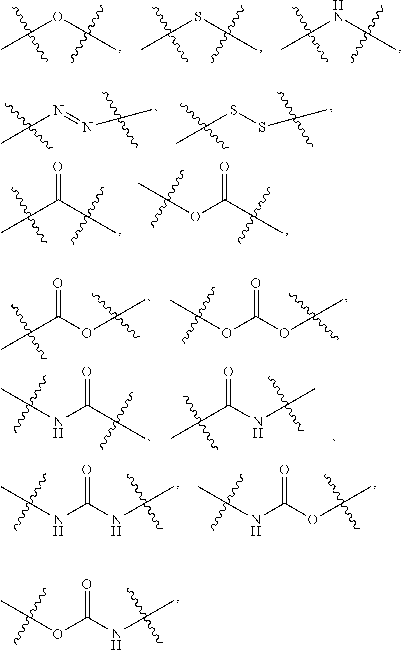

including combinations thereof.

The term “heterocycle,” as used herein, refers to ring structures that contain at least 1 noncarbon ring atom. A “heterocycle” for the purposes of this disclosure encompass from 1 to 4 heterocycle rings, wherein when the heterocycle is greater than 1 ring the heterocycle rings are joined so that they are linked, fused, or a combination thereof. A heterocycle may be aromatic or nonaromatic, or in the case of more than one heterocycle ring, one or more rings may be nonaromatic, one or more rings may be aromatic, or a combination thereof. A heterocycle may be substituted or unsubstituted, or in the case of more than one heterocycle ring one or more rings may be unsubstituted, one or more rings may be substituted, or a combination thereof. Typically, the noncarbon ring atom is N, O, S, Si, Al, B, or P. In the case where there is more than one noncarbon ring atom, these noncarbon ring atoms can either be the same element, or combination of different elements, such as N and O. Examples of heterocycles include, but are not limited to: a monocyclic heterocycle such as, aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide; and polycyclic heterocycles such as, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine. In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

The term “optionally substituted” refers to a functional group, typically a hydrocarbon or heterocycle, where one or more hydrogen atoms may be replaced with a substituent. Accordingly, “optionally substituted” refers to a functional group that is substituted, in that one or more hydrogen atoms are replaced with a substituent, or unsubstituted, in that the hydrogen atoms are not replaced with a substituent. For example, an optionally substituted hydrocarbon group refers to an unsubstituted hydrocarbon group or a substituted hydrocarbon group.

The term “substituent” refers to an atom or group of atoms substituted in place of a hydrogen atom. For purposes of this disclosure, a substituent would include deuterium atoms.

In general, “substitution” refers to an organic functional group defined below (e.g., an alkyl group) in which one or more bonds to a hydrogen atom contained therein are replaced by a bond to a non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise stated.

In some embodiments, a substituted group is substituted with one to six substituents. Examples of substituent groups include, but not limited to halogens (i.e. F, Cl, Br, and I), hydroxyls, alkoxy, alkenoxy, aryloxy, arylalkoxy, heterocyclyl, heterocyclylalkyl, heterocyclyloxy and heterocyclylalkoxy groups; carbonyls (oxo); carboxylates, esters, urethanes, oximes, hydroxylamines, alkoxyamines, aralkoxyamines, thiols, sulfides, sulfoxides, sulfones, sulfonyls, pentafluorosulfanyl (i.e. SF₅), sulfonamides, amines, N-oxides, hydrazines, hydrazides, hydrazones, azides, amides, ureas, amidines, guanidines, enamines, imides, isocyantes, isothiocyanates, cyanates, imines, nitro groups, nitriles, and the like.

The term “unsubstituted” with respect to hydrocarbons, heterocycles, and the like, refers to structures wherein the parent chain contains no substituents.

Extended-spectrum β-lactamase (ESBL)-producing Gram-negative bacteria (GNB) express enzymes that hydrolyze and inactivate most β-lactam antibiotics, including penicillins, cephalosporins, expanded-spectrum cephalosporins (including 3^{rd} and 4^{th}-generation agents), and monobactams. ESBL-producing Enterobacteriaceae were designated a “serious threat” by the Centers for Disease Control and Prevention (CDC) in their Antibiotic Resistance Threats report in 2013 and 2019, and a “critical priority” by the World Health Organization in their Global Priority List of Antibiotic-Resistant Bacteria in 2017. In 2017 there were an estimated 197,400 ESBL-producing Enterobacteriaceae infections in hospitalized patients in the United States, resulting in 9,100 deaths and $1.2 B in attributable healthcare costs. ESBL infections represent a major public health concern—infections occur in both healthcare and community settings, and their prevalence is increasing in the US and globally.

Urinary tract infections (UTIs) are one of the most common bacterial infections in community and healthcare settings, with a global incidence of roughly 150 million cases annually. UTIs caused by ESBL-producing GNB are a worldwide problem, with >20% prevalence in many regions around the world. Escherichia coli and Klebsiella pneumoniae from the family Enterobacteriaceae are the most common cause of UTIs, and the most prevalent ESBL-producing species. ESBL-producing E. coli and K. pneumoniae (ESBL-EK) are clinically problematic because they not only demonstrate resistance to most β-lactams, but are frequently multidrug-resistant. ESBL-EK are often co-resistant to fluoroquinolones, trimethoprim/sulfamethoxazole, and aminoglycosides, as well as β-lactams-antimicrobial agents which are used to empirically treat UTIs.⁷⁻¹¹ Once an ESBL-EK is identified as the etiologic pathogen of a UTI, only a limited number of treatment options remain; appropriate agents include carbapenems (currently only available as parenteral formulations in the US) and nitrofurantoin (only recommended for treatment of uncomplicated cystitis).

The rapid detection of ESBL-EK directly from urine samples of patients with UTIs remains an unmet clinical need. The current turnaround time for standard antimicrobial susceptibility testing methods that can identify these organisms is 2-3 days. Since there is no microbiological information available at the initial point of care to guide the selection of appropriate antimicrobial therapy, providers must rely on local empiric prescribing guidelines in conjunction with patient characteristics. In the case of complicated UTIs and pyelonephritis, empiric therapy guidelines typically do not specify agents effective against ESBL-producing GNB as first line therapy. As little as 24% of patients with ESBL-EK UTIs initially receive concordant antimicrobial therapy. On average, it takes two days longer to place patients with ESBL-EK UTIs on an appropriate drug compared to patients with non-ESBL-EK UTIs. In a study of hospitalized patients, ESBL-EK UTIs were associated with a longer length-of-stay (6 vs. 4 days) and a higher cost of care ($3658 more) than non-ESBL-EK UTIs. A diagnostic test that rapidly identifies UTIs caused by ESBL-producing GNB could provide clinicians with information that improves selection of effective initial therapy.

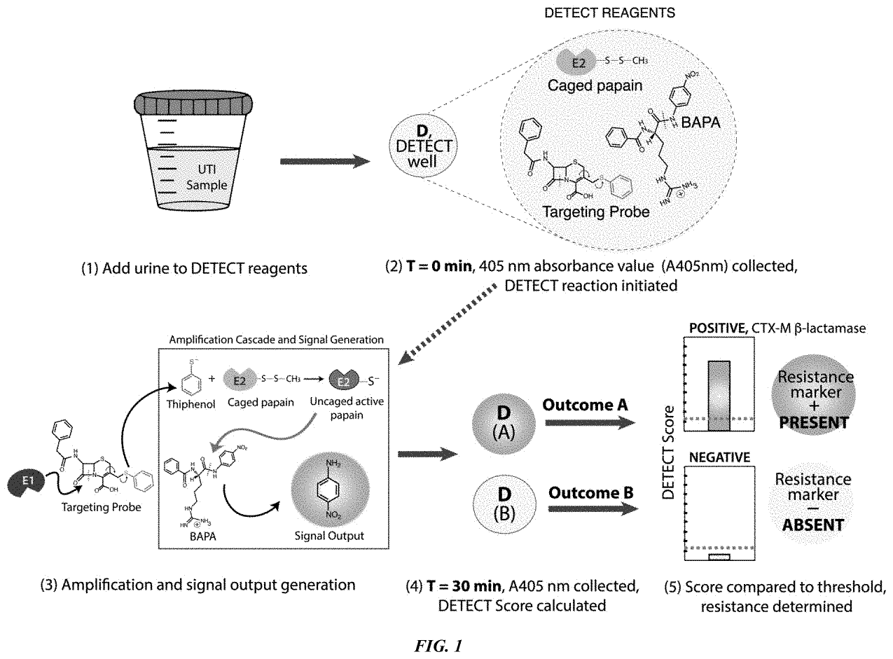

UTIs caused by ESBL-producing GNB cause significant clinical and economic burden, and there is an urgent need for rapid diagnostic tests that support the selection of appropriate therapy for treatment of these infections. A diagnostic test that rapidly identifies UTIs caused by ESBL-producing GNB directly from urine samples could provide clinicians with vital antimicrobial resistance information, allowing selection of appropriate antimicrobial therapy at the initial point of care. Such a test might improve patient outcomes and decrease the cost of care associated with these infections. Traditional PCR based tests have been challenging to develop for broad detection of ESBL-producing GNB, due to the sequence diversity exhibited by these β-lactamases. There are >150 CTX-M variants identified to date, that are subdivided into 5 groups based on sequence homology. Additionally, while all CTX-Ms are considered ESBLs, some enzyme families encompass sequence variants that mediate very different β-lactam resistance profiles. For example, the TEM and SHV β-lactamase families consist of ESBL and non-ESBL variants which may differ in sequence by as little as one amino acid. Therefore, technologies or testing methods that detect phenotypic (AST) or enzymatic activity of these β-lactamases should provide the greatest utility and versatility for detection of these diverse resistance enzymes. Biochemical-based diagnostic tests hold great promise in this regard, and can offer other advantages that make them suitable for widespread point-of-care clinical use, including simplicity, scalability, low cost, and even little to no instrumentation requirements. However, developing point of care tests that can identify ESBL producing GNB directly from patient samples is challenging because of the low number of bacteria and the complex milieu in urine samples. To overcome the sensitivity limitations of traditional biochemical-based approaches for β-lactamase detection, we developed a dual-enzyme trigger-enabled cascade technology. A method disclosed herein connects a target β-lactamase to a disulfide-caged enzyme amplifier (papain) via a compound of the disclosure that eliminates a triggering unit (thiophenol) upon b-lactamase-mediated hydrolysis, releasing the caged papain that then generates a colorimetric signal output (see FIG. 1). As shown herein, the amplification power of the methods disclosed herein relative to the standard chromogenic probe, nitrocefin, in side-by-side analyses of β-lactamase enzymes and β-lactam-resistant clinical isolates producing several common β-lactamases.

The compounds and methods disclosed herein allow for the identification of UTIs caused by CTX-M-producing GNB in as little as 30 min. The compounds and methods disclosed herein were used to identify UTIs in three systems with increasing complexity: first with purified recombinant β-lactamases, second with β-lactamase-producing clinical isolates, and third with clinical urine samples. The methods disclosed herein is composed of two tiers—a targeting tier and an amplification/signal output tier—which are connected in series via the trigger-releasing β-lactamase probe. In the studies presented herein, the selective hydrolysis of the β-lactamase probe by CTX-Ms was first explored with a panel of diverse recombinant β-lactamases. In contrast to traditional kinetic approaches that are performed using higher concentrations of enzyme and substrate, the LODs of the methods were defined for each β-lactamase as a measure of sensitivity towards a specific variant. LOD values of the compounds and methods disclosed herein revealed a strong proclivity of β-lactamase probe towards CTX-M β-lactamases, with the average LOD for the four tested CTX-M variants (0.041 nM) being 42-times lower than the average LOD of the non-CTX-M β-lactamases tested (excluding CMY and OXA). Similarly, the compounds and methods disclosed herein were found to be sensitive towards CMY (a chromosomal or plasmid-mediated AmpC), which generated the same LOD (0.041 nM) as the average of the CTX-M variants. The selectivity of the compounds and methods of the disclosure were further demonstrated in CTX-M and CMY-producing clinical isolates, which on average generated higher DETECT Scores than GNB producing other β-lactamases or GNB demonstrating susceptibility to β-lactams.

Clavulanic acid is a known β-lactamase inhibitor that typically inhibits the enzymatic activity of traditional ESBLs but not AmpC β-lactamases. As a means to resolve CTX-M from CMY-producing GNB, the use of a β-lactamase inhibitor with the compounds and methods disclosed herein were explored. The comparison of scores generated from the compounds and methods disclosed herein alone vs. compounds and methods disclosed herein with clavulanic acid, indicated that use of a β-lactamase inhibitor with the compounds and methods of the disclosure were an effective way to differentiate between bacteria producing these enzymes. Scores from CMY-producing isolates were minimally affected by addition of clavulanic acid, while scores from CTX-M-producing isolates were widely affected. It is envisioned that any number of known β-lactamase inhibitors can be used with the compounds and methods disclosed herein, as a means to enable further specificity or resolution of β-lactamases in the system.

In the clinical urine studies presented herein, the compounds and methods of the disclosure were found to be robust and maintained selectivity towards CTX-M-producing bacteria. Many of the false-positive results in urine could be attributed to a high CFU/mL of TEM-1-producing or AmpC-producing GNB. When tested as individual isolates using the compounds and methods disclosed herein (where number of CFU are controlled), the TEM-1 or cAmpC-producing GNB tested correctly negative. It is postulated herein that used of a CTX-M-specific inhibitor with the compounds and methods of the disclosure, as opposed to clavulanic acid, would have broader utility in the resolution of CTX-Ms from other β-lactamases. TEM-1 is also supposed to demonstrate susceptibility to the effects of clavulanic acid, so this inhibitor would likely not be effective at differentiating scores from TEM-1 vs. CTX-Ms. It is further postulated herein that cross-reactivity with other β-lactamases could be minimized by making various design changes in the β-lactamase-targeting probe as further described herein. For example, the β-lactamase-targeting probe can be modified so that it better resembles other β-lactam scaffolds that are preferentially hydrolyzed by target enzymes. Thus, it is expected that the various compounds described herein would have increase specificity towards the desired targeted β-lactamases than other compounds known in the art.

In the preliminary studies presented herein, the compounds and methods disclosed herein correctly identified at least 91% of the microbiologically-defined UTIs with CTX-M-producing GNB. It was found than only one reference-positive urine sample tested false-negative in the DETECT assay of the disclosure; this sample contained a CTX-M-15-producing K. pneumoniae at an estimated 10⁴-10⁵ CFU/mL. Since the clinical isolate itself tested correctly-positive in the methods disclosed herein, the CFU in the original urine sample was likely below the current LOD of the compounds and methods disclosed herein in urine. Based on the CFU/mL estimates in samples that were true-positives, and based on previous LOD experiments with a CTX-M-producing clinical isolate, it was estimated that the current assay has an average LOD concentration of 10⁶ CFU/mL of CTX-M-producing GNB in urine. The LOD is within a clinically relevant concentration range for UTI. It is expected that the LOD of the DETECT assay disclosed herein could be adjusted for synchronization with microbiological cutoffs, through different modifications of the compounds and methods disclosed herein. The disclosure provides in various embodiments disclosed herein, modification of the amplification/signal output tier of the compounds and methods of the disclosure; modification of the papain enzyme amplifier for greater catalytic efficiency; and/or modification of the colorimetric substrate to yield a higher turnover rate are viable options.

While none of the TEM and SHV ESBL-producing GNB identified in the urine study were MDR, 91% of the CTX-M-producing GNB were MDR, highlighting the importance of specific identification of CTX-M-producing bacteria. The CTX-M-producing isolates mainly demonstrated resistance to the following agents/classes (besides the β-lactams): ciprofloxacin and levofloxacin (fluoroquinolones), trimethoprim/sulfamethoxazole (folate-pathway inhibitors), and gentamicin and tobramycin (aminoglycosides). Six (60%) of 10 CTX-M-producing/MDR isolates were dually resistant to the fluoroquinolones and trimethoprim/sulfamethoxazole; both are important empirical agents for the treatment of complicated UTI and pyelonephritis (as are expanded-spectrum β-lactams) (cite).

The compounds and methods of the disclosure has been validated against a wide variety of ESBL-EK and non-ESBL-EK clinical isolates. Since other species of bacteria were also identified in urine samples—including an ESBL-producing P. mirabilis—the DETECT system requires further testing against these other species of bacteria (where possible with ESBL-producing and non-producing isolates) to establish common score trends. Likewise, additional β-lactamase variants (including cAmpC enzymes) commonly encountered in urine samples should be assessed for LOD in recombinant β-lactamase form. These experiments will further elucidate the selectivity the compounds and methods disclosed herein, and help define its limitations. While we predict that any GNB species producing a CTX-M will be identifiable by DETECT, further experiments are required to validate this theory.

The compounds and methods of the disclosure has the following features: the assay is easy to perform; urine sample processing is not needed; all reagents can be stored in liquid form, such that the only steps required to perform the assay in its current 96-well plate format including, but not limited to: pipetting reagents into wells, pipetting samples into wells, setting up the plate on a microplate reader for a 0 min and 30 min read, then calculating a score. In view of the following assay steps, it is clear that implementation of the method can be carried out by personnel at the bench, or be carried out using semi-automated or fully-automated devices. Being about to run the compounds and methods of the disclosure in a semi-automated or fully-automated fashion would mitigate operator error and inter-operator variability, limit test complexity, and limit the total hands-on time required to perform this test, which would encourage wider adoptability. The compounds and methods of the disclosure can be used at the point of care, thereby providing actionable results in a time-frame that positively impacts the identification of a therapeutically effective first antimicrobial agent that can be prescribed to a patient. For use of point of care applications, the device incorporating the compounds and methods disclosed herein would ideally need to be small, robust, and simple to use. The compounds and methods of the disclosure have a simple colorimetric output, which should make integration into a device more straightforward and enable flexible format options. The colorimetric output of the compounds and methods of the disclosure can be read by a microplate reader, but could also be read by other spectrophotometric devices or even by a device application (e.g., mobile phone app). Enhancement of the colorimetric signal can also enable accurate detection by eye.

The compounds disclosed herein were rapidly hydrolyzed by targeted β-lactamases studied herein. The results demonstrate significant preference of the compounds of the disclosure towards a subclass of ESBLs known as CTX-M-type-lactamases. For example, certain compounds of the disclosure were hydrolyzed by an ESBL to release a trigger unit that activates an enzymes amplifier, initiating an amplification cascade event that generates a colorimetric signal output indicating the presence of an ESBL. The ESBL-detecting compounds can be applied as a diagnostic reagent to detect ESBL-producing pathogens and direct care of patients.

In various aspects, the disclosure provides compounds and methods for detecting antimicrobial resistance via the identification of β-lactamase variants that are responsible for the enzyme mediated resistance mechanism present in gram-negative and gram-positive bacteria. The compounds provided herein can be formulated into an amplification assay composition that are useful in the disclosed methods. Also provided is the use of the compounds in preparing assay formulations for the amplification method.

In a particular embodiment, the disclosure provides for a compound that comprises a structure of Formula I:

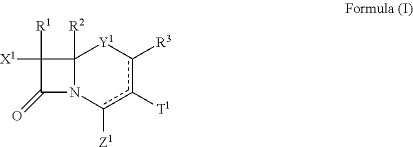

or a salt, stereoisomer, tautomer, polymorph, or solvate thereof, wherein:

T¹ is a benzenethiol containing group or Z², wherein if T¹ is Z², then Z¹ is T²;

Z¹ is a carboxylate, a carbonyl, an ester, an amide, a sulfone, a sulfonamide, a sulfonyl, —S(O)₂OH or T², wherein if Z¹ is T², then T¹ is Z²;

T² is a benzenethiol containing group;

Z² is a carboxylate, a carbonyl, an ester, an amide, a sulfone, a sulfonamide, a sulfonyl, or —S(O)₂OH;

X¹ is

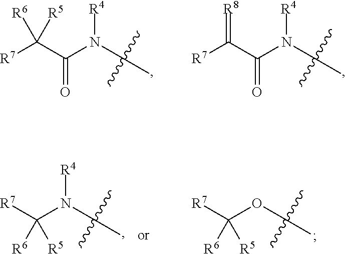

Y¹ is

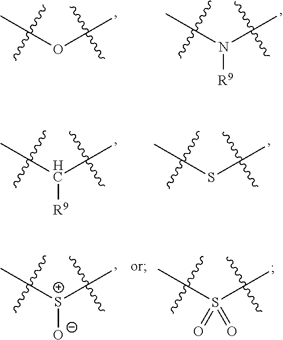

R¹-R⁶, and R⁹-R¹¹ are each independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle;

R⁷ is an optionally substituted (C₅-C₇) cycloalkyl, optionally substituted aryl, optionally substituted benzyl, or optionally substituted heterocycle; and

R⁸ is

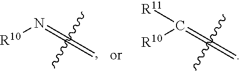

In a further embodiment, T¹ is Z² or a benzenethiol containing group selected from the group consisting of:

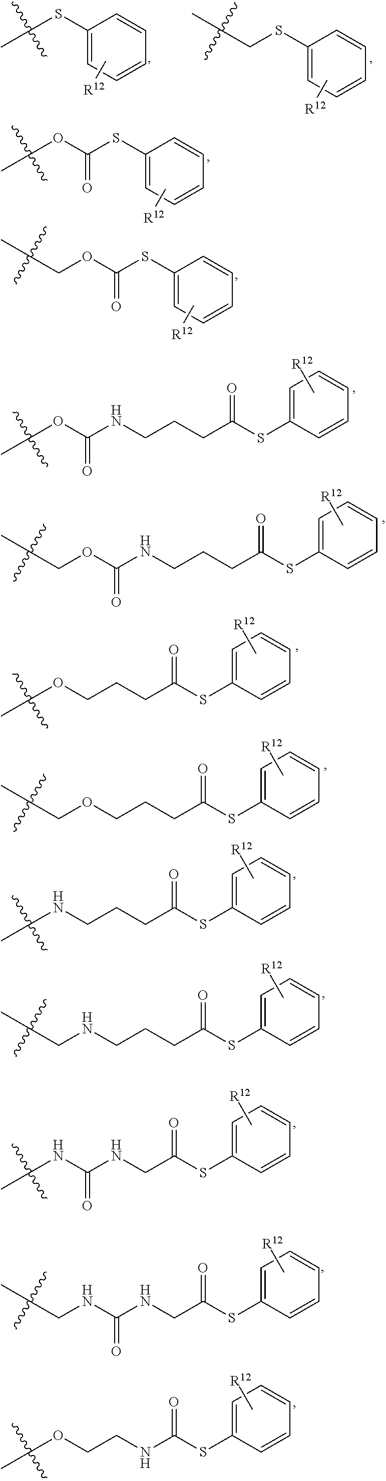

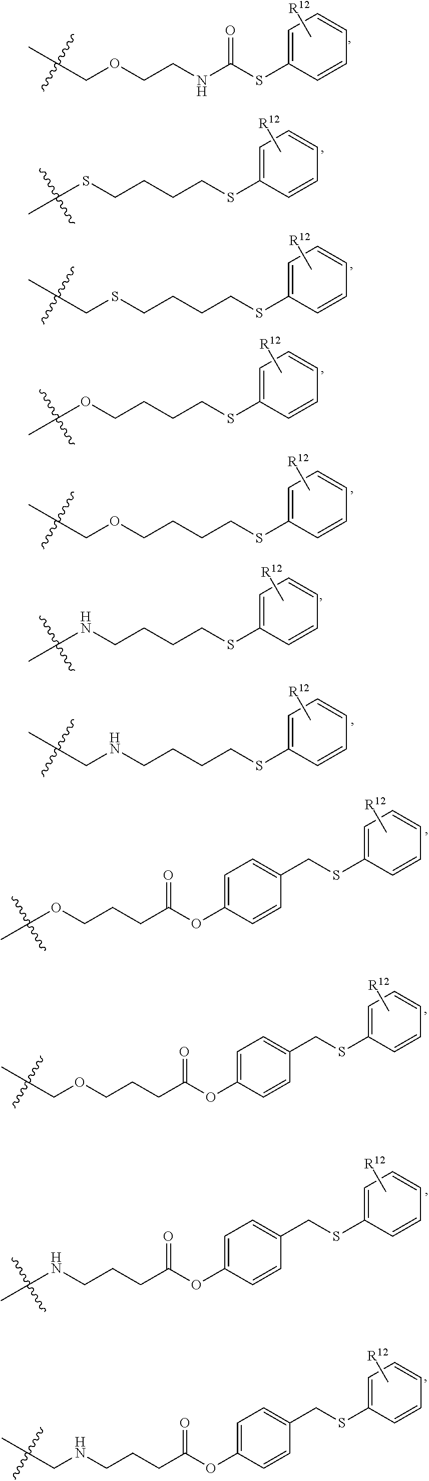

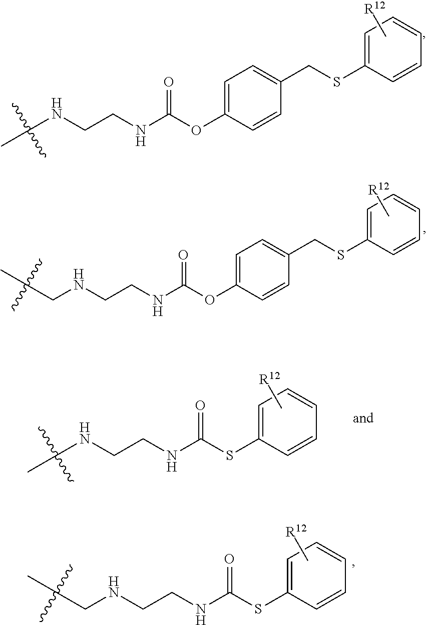

wherein R¹² is H D, alkoxy, hydroxyl, ester, amide, aryl, heteroaryl, nitro, cyanate, nitrile, or halo. In yet a further embodiment, T² is a benzenethiol containing group selected from the group consisting of:

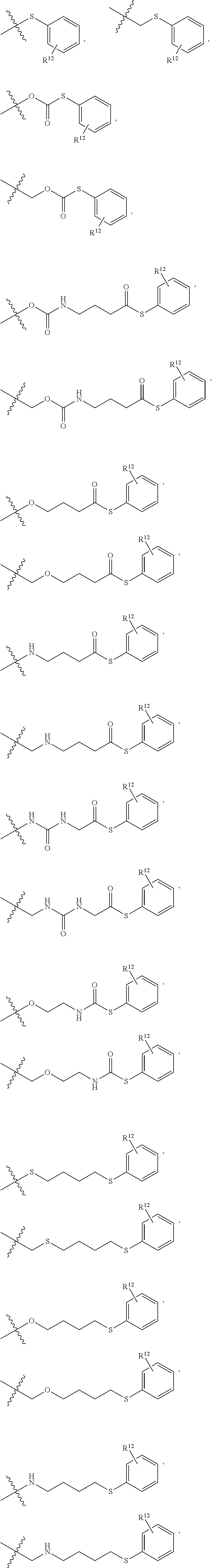

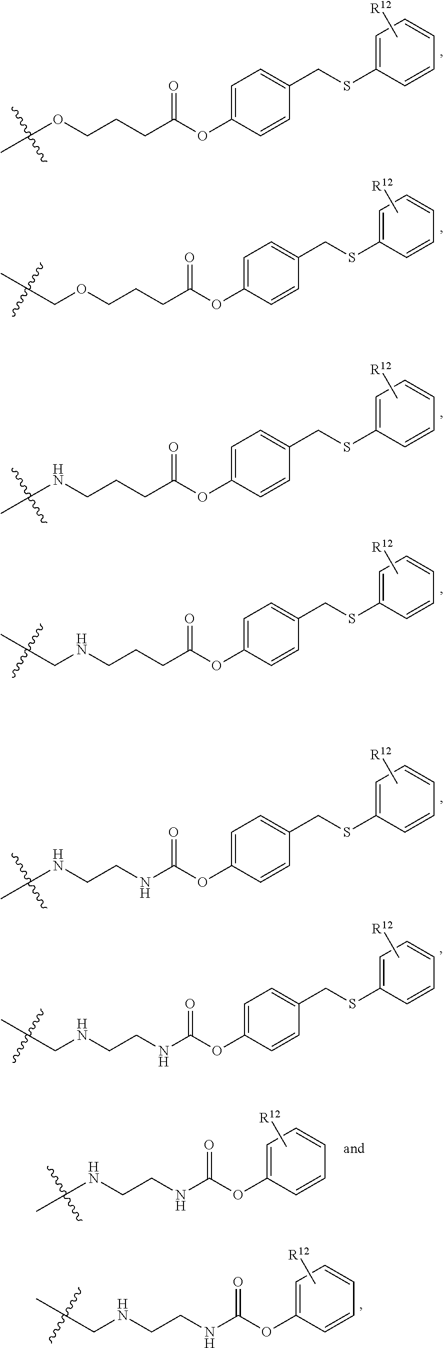

wherein R¹² is H, D, alkoxy, hydroxyl, ester, amide, aryl, heteroaryl, nitro, cyanate, nitrile, or halo. In another embodiment, R⁷ is selected from the group consisting of:

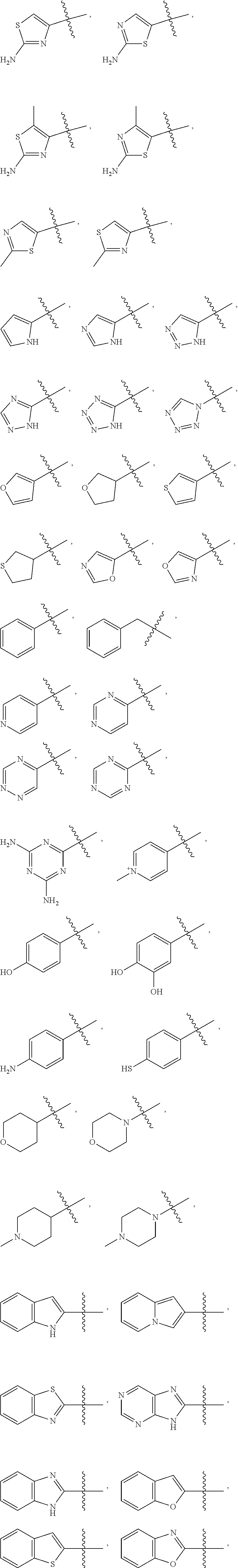

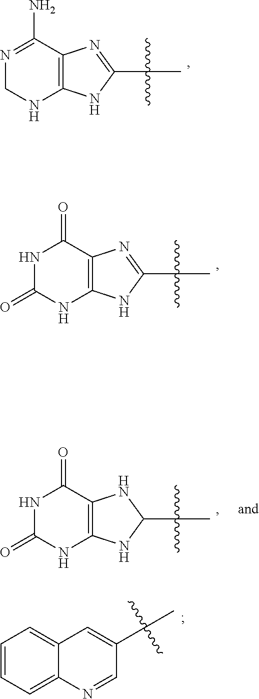 [SMILES: *c1nc2c([nH]1)NCN=C2N ; *c1nc2c(=O)[nH]c(=O)[nH]c2[nH]1 ; *C1Nc2[nH]c(=O)[nH]c(=O)c2N1 ; *c1cnc2ccccc2c1]

In a certain embodiment, the compound of Formula I does not have a structure of:

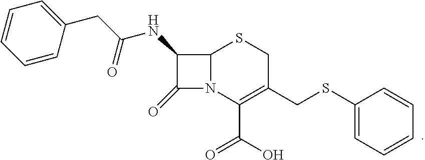 [SMILES: O=C(Cc1ccccc1)N[C@@H]1C(=O)N2C(C(=O)O)=C(CSc3ccccc3)CSC12]

In a further embodiment, the disclosure provides for a compound that comprises a structure of Formula I(a):

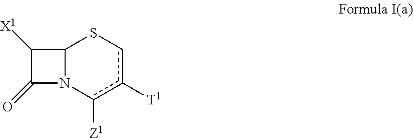

or a salt, stereoisomer, tautomer, polymorph, or solvate thereof, wherein:

T¹ is a benzenethiol containing group or Z², wherein if T¹ is Z², then Z¹ is T²;

Z¹ is a carboxylate, a carbonyl, an ester, an amide, a sulfone, a sulfonamide, a sulfonyl, —S(O)₂OH or T², wherein if Z¹ is T², then T¹ is Z²;

T² is a benzenethiol containing group;

Z² is a carboxylate, a carbonyl, an ester, an amide, a sulfone, a sulfonamide, a sulfonyl, or —S(O)₂OH;

X¹ is

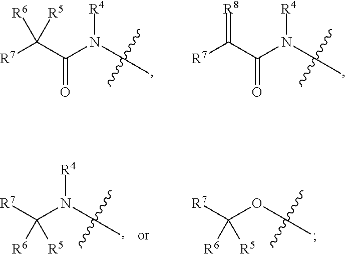

R⁴, R⁵, and R¹⁰ are independently an H or a (C₁-C₆)alkyl;

R⁶ is an H, or an amine;

R⁷ is an optionally substituted (C₅-C₇) cycloalkyl, optionally substituted aryl, optionally substituted benzyl, or optionally substituted heterocycle;

R⁸ is

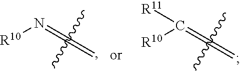

and

R⁹ is a hydroxyl or an (C₁-C₃)alkoxy. In a certain embodiment, the compound of Formula I(a) does not have a structure of:

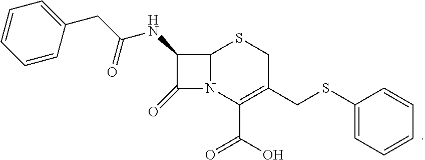 [SMILES: O=C(Cc1ccccc1)N[C@@H]1C(=O)N2C(C(=O)O)=C(CSc3ccccc3)CSC12]

In a particular embodiment, the disclosure provides a compound that comprises a structure of Formula I(b):

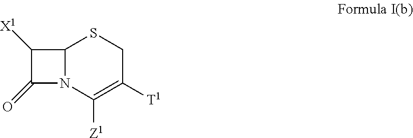

or a salt, stereoisomer, tautomer, polymorph, or solvate thereof, wherein:

T¹ a benzenethiol containing group selected from the group consisting of:

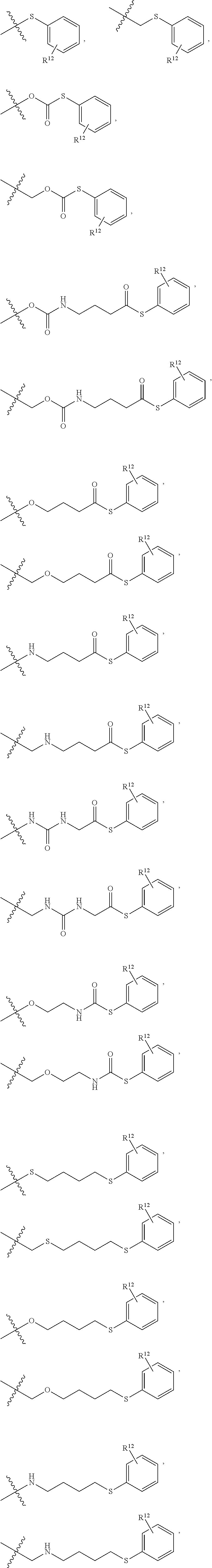

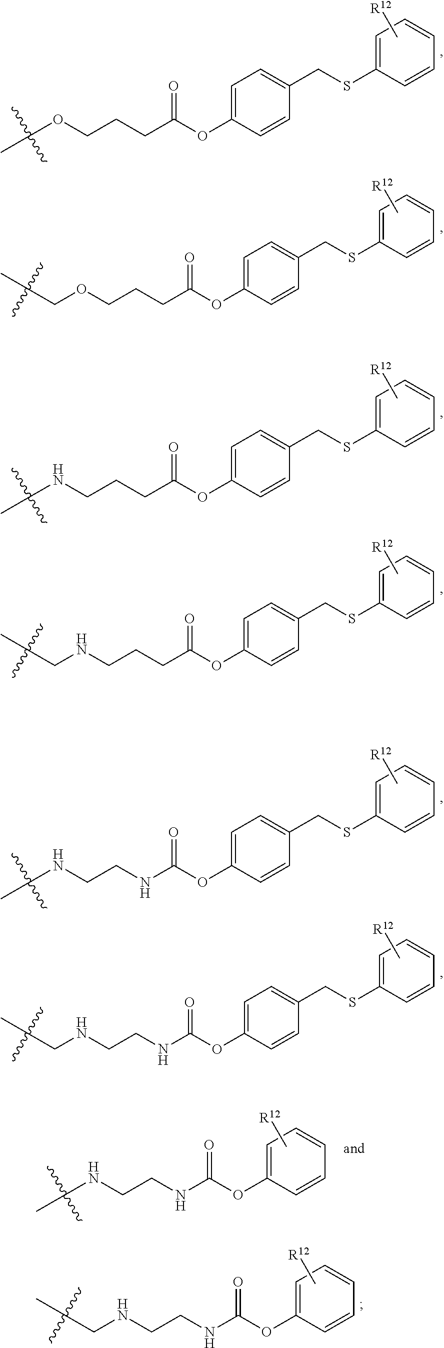

Z¹ is a carboxylate, a carbonyl, an ester, an amide, a sulfone, a sulfonamide, a sulfonyl, —S(O)₂OH or T²;

X¹ is

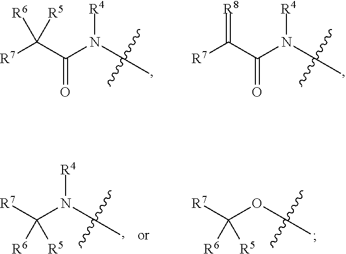

R⁴, R⁵, and R¹⁰ are independently an H or a (C₁-C₆)alkyl;

R⁶ is an H, or an amine;

R⁷ is an optionally substituted aryl, optionally substituted benzyl, or optionally substituted heterocycle;

R⁸ is

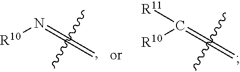

R⁹ is a hydroxyl or an (C₁-C₃)alkoxy;

R¹² is H, D, alkoxy, hydroxyl, ester, amide, aryl, heteroaryl, nitro, cyanate, nitrile, or halo.

In a further embodiment, R⁷ is selected from the group consisting of:

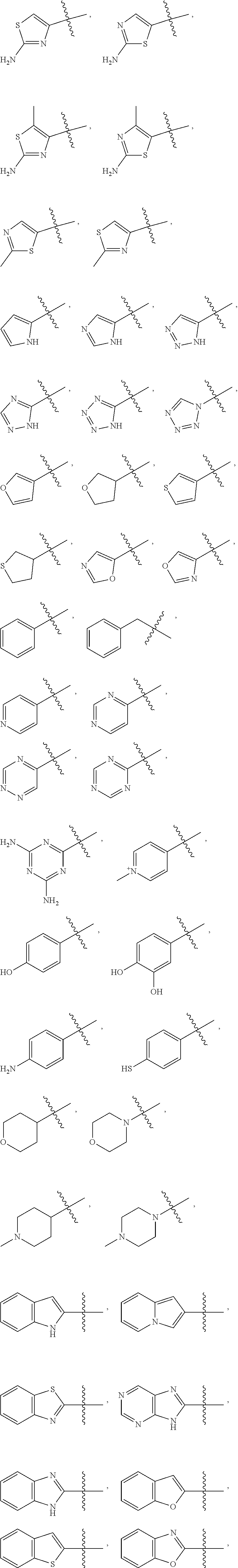

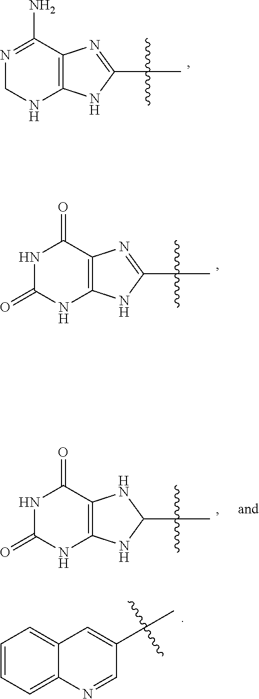 [SMILES: *c1nc2c([nH]1)NCN=C2N ; *c1nc2c(=O)[nH]c(=O)[nH]c2[nH]1 ; *C1Nc2[nH]c(=O)[nH]c(=O)c2N1 ; *c1cnc2ccccc2c1]

In a particular embodiment, the compound of Formula I(b) does not have a structure of:

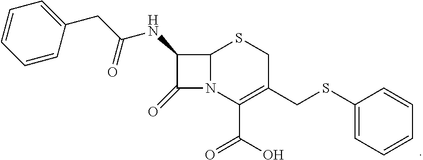 [SMILES: O=C(Cc1ccccc1)N[C@@H]1C(=O)N2C(C(=O)O)=C(CSc3ccccc3)CSC12]

In a further embodiment, the disclosure provides a compound that comprises a structure of Formula I(c):

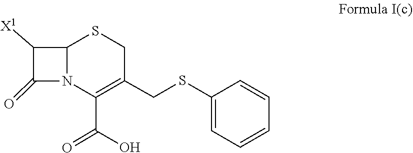

### X¹ is

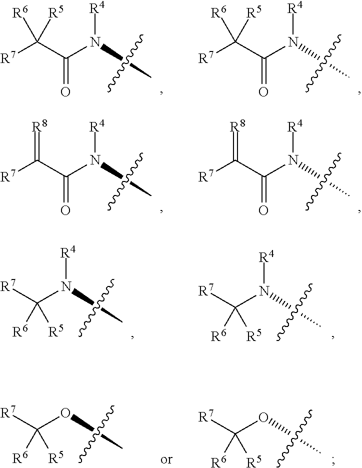

R⁴, R⁵, and R¹⁰ are independently an H or a (C₁-C₆)alkyl;

R⁶ is an H, or an amine;

R⁷ is selected from the group consisting of:

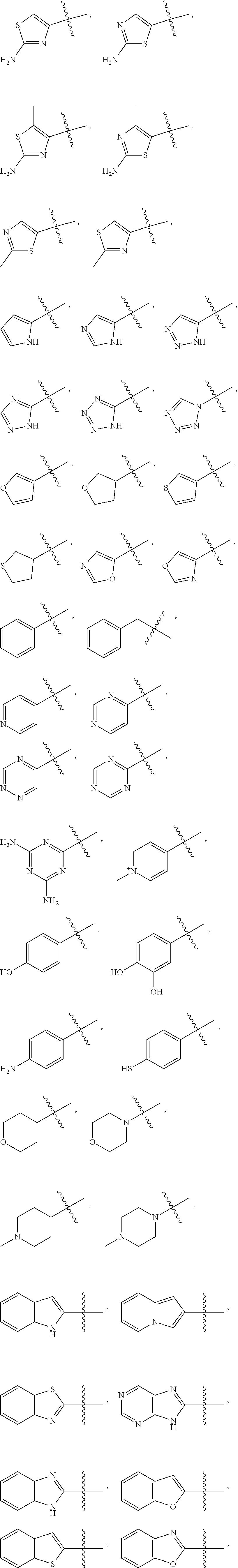

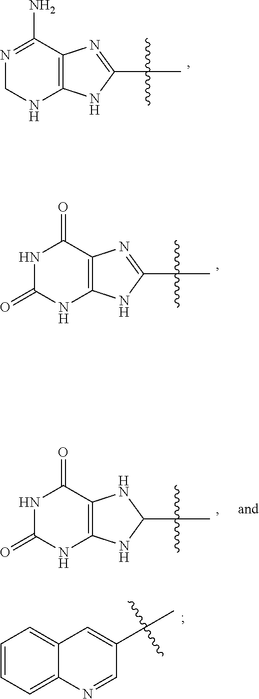 [SMILES: *c1nc2c([nH]1)NCN=C2N ; *c1nc2c(=O)[nH]c(=O)[nH]c2[nH]1 ; *C1Nc2[nH]c(=O)[nH]c(=O)c2N1 ; *c1cnc2ccccc2c1]

R⁸ is

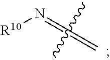

and

R⁹ is

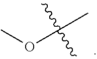 [SMILES: *OC]

In a certain embodiment, the compound of Formula I(c) does not have a structure of:

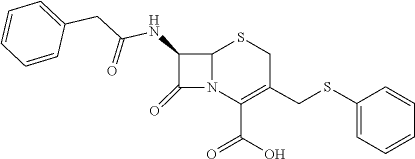 [SMILES: O=C(Cc1ccccc1)N[C@@H]1C(=O)N2C(C(=O)O)=C(CSc3ccccc3)CSC12]

### (i.e., if X¹ is

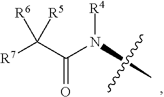

then R⁷ is not

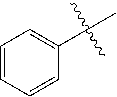 [SMILES: *c1ccccc1]

when R⁴-R⁶ are H).

In a further embodiment, the disclosure provides for a compound of Formula I having a structure selected from:

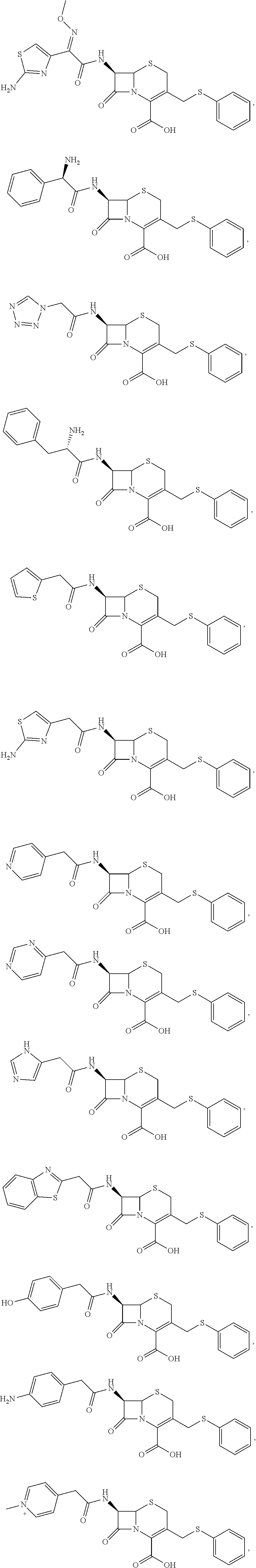

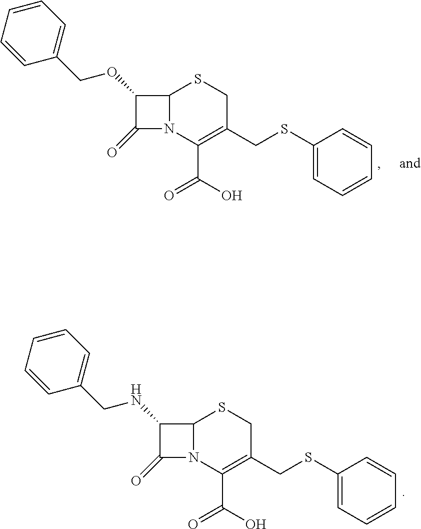 [SMILES: O=C(O)C1=C(CSc2ccccc2)CSC2[C@@H](OCc3ccccc3)C(=O)N12 ; O=C(O)C1=C(CSc2ccccc2)CSC2[C@@H](NCc3ccccc3)C(=O)N12]

In a particular embodiment, the disclosure provides a compound that comprises a structure of Formula II:

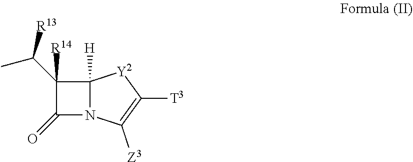

or a salt, stereoisomer, tautomer, polymorph, or solvate thereof, wherein:

Y² is

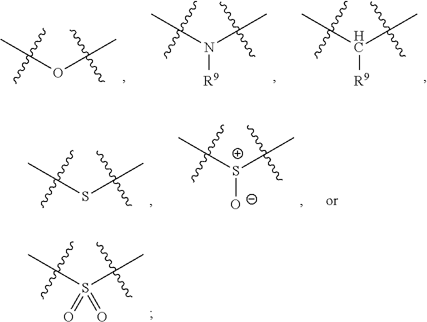

R⁹, R¹³ and R¹⁴ are independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle;

Z³ is a carboxylate, a carbonyl, an ester, an amide, a sulfone, a sulfonamide, a sulfonyl, or —S(O)₂OH; and

T³ is a benzenethiol containing group. In a further embodiment, T³ is a benzenethiol containing group selected from the group consisting of:

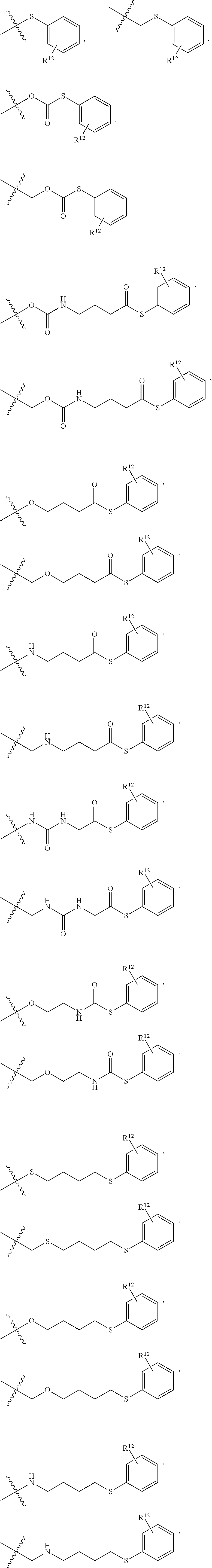

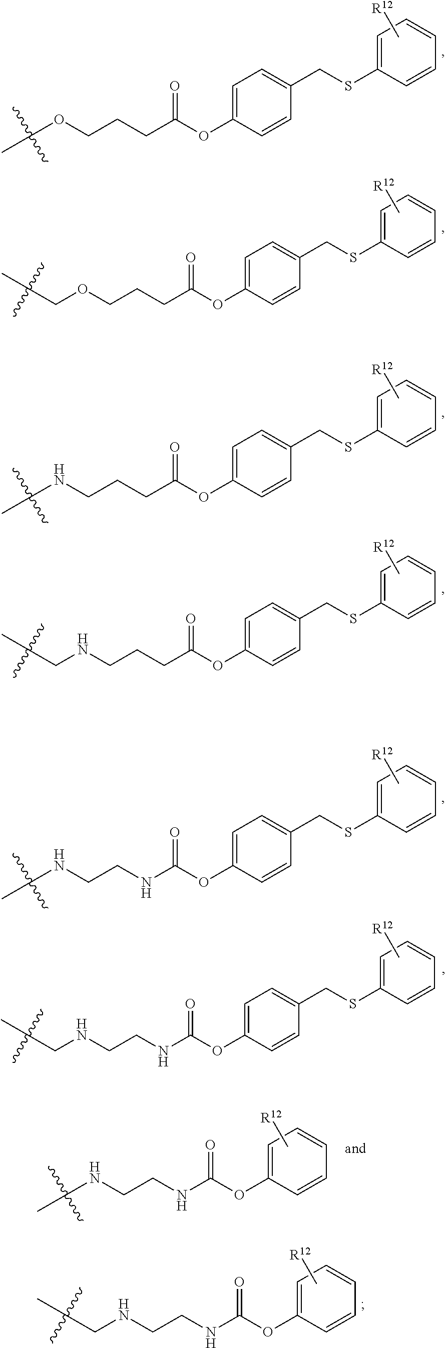

and

R¹² is H, D, alkoxy, hydroxyl, ester, amide, aryl, heteroaryl, nitro, cyanate, nitrile, or halo.

In another embodiment, the disclosure provides a compound that comprises a structure of Formula II(a):

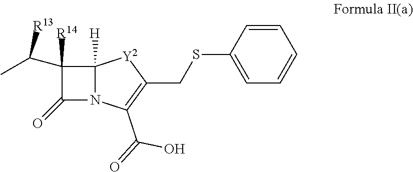

or a salt, stereoisomer, tautomer, polymorph, or solvate thereof, wherein:

Y² is

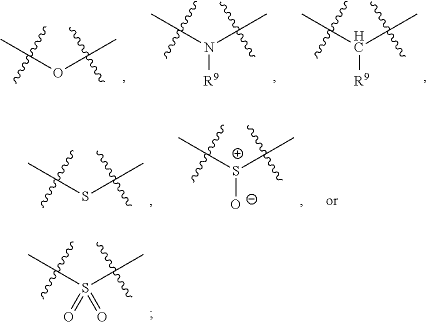

R⁹, R¹³ and R¹⁴ are independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle.

In yet another embodiment, the disclosure provides a compound that comprises a structure of Formula II(b):

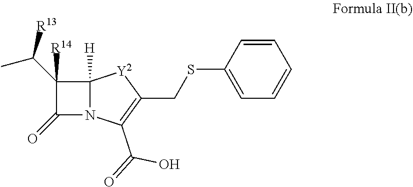

or a salt, stereoisomer, tautomer, polymorph, or solvate thereof, wherein:

Y² is

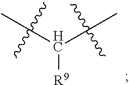

R⁹, R¹³ and R¹⁴ are independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, and optionally substituted (C₁-C₆)alkyl.

In a further embodiment, the disclosure provides for a compound of Formula II having a structure selected from:

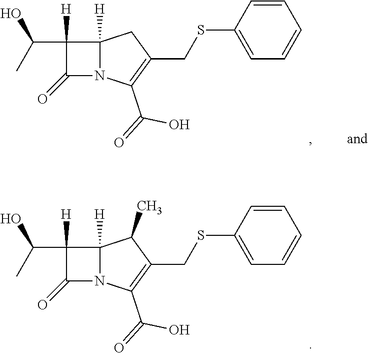 [SMILES: C[C@@H](O)[C@H]1C(=O)N2C(C(=O)O)=C(CSc3ccccc3)C[C@H]12 ; C[C@@H](O)[C@H]1C(=O)N2C(C(=O)O)=C(CSc3ccccc3)[C@H](C)[C@H]12]

In a further embodiment, a compound disclosed herein is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

In a further embodiment, a compound disclosed herein is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 1⁰% or less by weight of any other diastereomer.

A compound disclosed herein may be enantiomerically pure, such as a single enantiomer or a single diastereomer, or be stereoisomeric mixtures, such as a mixture of enantiomers, a racemic mixture, or a diastereomeric mixture. Conventional techniques for the preparation/solation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When a compound disclosed herein contains an acidic or basic moiety, it may also be disclosed as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The disclosure provides methods to detect the presence of one or more target β-lactamases in a sample by using the compounds disclosure herein. In a particular embodiment, a method disclosed herein has the step of: adding reagents to a sample suspected of comprising one or more target β-lactamases, wherein the reagents comprise: (i) a compound of the disclosure; (ii) a chromogenic substrate for a cysteine protease; and (iii) a cagedinactive cysteine protease; and (iv) optionally, an inhibitor to specific type(s) or class(es) of β-lactamases. For (ii), (iii) and (iv) these substrates, enzymes and inhibitors can be made up in the buffers as described in the examples section herein. The sample used in the methods typically is obtained from a subject, but the sample may also come from other sources, such as a water sample, an environmental sample, a wastewater sample, etc. Samples obtained from the subject can come from various portions of the body. For example, the sample can be a blood sample, a urine sample, a cerebrospinal fluid sample, a saliva sample, a rectal sample, a urethral sample, or an ocular sample. In regards to the latter three samples these samples can be obtained by swabbing the various regions. In a particular embodiment, the sample is a blood or urine sample. The subject that the sample is obtained from can be from any animal, including but not limited to, humans, primates, cats, dogs, horses, birds, lizards, cows, pigs, rabbits, rats, mice, sheep, goats, etc. In a particular embodiment, the sample is obtained from a human patient that has or is suspected of having a bacterial infection. For example, the human patient may have or be suspected of having a urinary tract infection, sepsis, or other infection.

In regards to targeted β-lactamases, the compounds of the disclosure can be used to target every known class of β-lactamases, including subtypes thereof. For example, the compound and methods disclosed herein can be used to delineate and detect the presence of penicillinases, extended-spectrum β-lactamases (ESBLs), inhibitor-resistant β-lactamases, AmpC-type β-lactamases, and carbapenemases. Extended-spectrum β-lactamases or ESBLs, in particular, can be targeted by the compounds and methods disclosed herein. For example, the compounds and methods disclosed herein can detect TEM β-lactamases, SHV β-lactamases, CTX-M β-lactamases, OXA β-lactamases, PER β-lactamases, VEB β-lactamases, GES β-lactamases, IBC β-lactamases. As shown in the studies presented herein various compounds disclosed herein can detect CTX-M β-lactamases with high specificity. The compounds and methods disclosed herein and also detected the various subtypes of carbapenemases, including but not limited to, metallo-β-lactamases, KPC β-lactamases, Verona integron-encoded metallo-β-lactamases, oxacillinases, CMY β-lactamases, New Delhi metallo-β-lactamases, Serratia marcescens enzymes, IMIpenem-hydrolysing β-lactamases, NMC β-lactamases and CcrA β-lactamases. For example, the studies presented herein demonstrates that various compounds of the disclosure can detect CMY β-lactamases and KPC β-lactamases with high specificity. In a particular embodiment, compounds disclosed herein can detect CTX-M β-lactamases, CMY β-lactamases and KPC β-lactamases with high specificity. Further delineation as to specific target s-lactamases in a sample can be determined by use of β-lactamase inhibitors, as is further described herein.

A chromogenic substrate typically refers to a colorless chemical, that an enzyme can convert into a deeply colored chemical. In a particular embodiment, the chromogenic substrate is a substrate for a cysteine protease, as further disclosed herein. Once acted on by the enzyme (e.g., cysteine protease) the cleaved product can be quantified based upon measuring light absorbance at a certain wavelength, e.g., 400 nm, 405 nm, 410 nm, 415 nm, 420 nm 425 nm, 430 nm, 435 nm, 440 nm, 445 nm, 450 nm, 455 nm, 460 nm, 465 nm, 470 nm, 475 nm, 480 nm, 485 nm, 490 nm, 495 nm, 500 nm, or a range that includes or is in-between any two of the foregoing light absorbance values. For example, cleavage products for: Nα-benzoyl-L-arginine 4-nitroanilide hydrochloride (BAPA) can be quantified by measuring light absorbance at 405 nm; L-pyroglutamyl-L-phenylalanyl-L-leucine-p-nitroanilide (PFLNA) can be quantified by measuring light absorbance at 410 nm; azocasein can be quantified by measuring light absorbance at 440 nm; pyroglutamyl-L-phenylalanyl-L-leucine-p-nitroanilide can be quantified by measuring light absorbance at 410 nm. Any number of devices can be used to measure light absorption, including microplate readers, spectrophotometers, scanners, etc. The light absorption of the sample can be measured at various time points, e.g., 0 min, 5 min, 15 min, 20 min, 25 min, 30 min, 35 min, 40 min, 45 min, 50 min, 55 min, 60 min, 70 min, 80 min, 90 min, 100 min, 110 min, 120 min, 240 min, or a range that includes or is in-between any two of the foregoing time points. For example, the light absorption of the sample can be measured at 0 min and 30 min, or at various time points in between to establish a reaction rate.

Cysteine proteases, also known as thiol proteases, are enzymes that degrade proteins. These proteases share a common catalytic mechanism that involves a nucleophilic cysteine thiol in a catalytic triad or dyad. Cysteine proteases are commonly encountered in fruits including the papaya, pineapple, fig and kiwifruit. Caged or inactive cysteine proteases refers to cysteine proteases that can be activated by removal of an inhibitory segment or protein. For example, a caged/inactive papain would include papapin-S—SCH₃, whereby the inhibiting thiol segment can be removed by the breaking of the disulfide bond. Examples of cysteine proteases that can be used in the method disclosed herein, include, but are not limited to, papain, bromelain, cathepsin K, calpain, caspase-1, galactosidase, seperase, adenain, pyroglutamyl-peptidase I, sortase A, hepatitis C virus peptidase, sindbis virus-type nsP2 peptidase, dipeptidyl-peptidase VI, deSI-1 peptidase, TEV protease, amidophosphoribosyl transferase precursor, gamma-glutamyl hydrolase, hedgehog protein, and dmpA aminopeptidase. In a particular embodiment, a caged/inactive papain (e.g., papain-S—SCH₃) is used in the methods disclosed herein, in combination with a chromogenic substrate for papain (e.g., BAPA). Caged/inactive cysteine proteases can generally be reactivated by reacting with low molecular weight thiolate anions (e.g., benzenethiolate anions) or inorganic sulfides. In a particular embodiment, the compounds of the disclosure are a substrate for one or more targeted β-lactamases and release a benzenethiolate anion product:

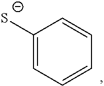

which then acts as a reaction amplifier by activating caged/inactive cysteine proteases (e.g., see FIG. 1).

For a method of the disclosure, the light absorbance of a sample can be compared with an experimentally determined threshold value to determine whether the targeted β-lactamase is present in the sample. For example, if the sample absorbance value is more than the experimentally determined threshold value, then the sample likely comprises a targeted β-lactamase. Alternatively, if the sample absorbance value is less than the experimentally determined threshold value, then sample likely does not comprise a targeted β-lactamase. Methods to generate an experimentally determined threshold value are taught in more detail herein, in the Examples section. Briefly, the experimentally determined threshold value can be determined by analysis of a receiver operating characteristic (ROC) curve generated from an isolate panel of bacteria that produce β-lactamases, wherein the one of more target β-lactamases have the lowest limit of detection (LOD) in the isolate panel.

The disclosure further provides for the use of one or more β-lactamase inhibitors with the compounds and method disclosed herein. β-lactamase inhibitors designed to bind at the active site of β-lactamases, which are frequently β-lactams. Two strategies for β-lactamase inhibitors are used: (i) create substrates that reversibly and/or irreversibly bind the enzyme with high affinity but form unfavorable steric interactions as the acyl-enzyme or (ii) develop mechanism-based or irreversible “suicide inhibitors”. Examples of the former are extended-spectrum cephalosporins, monobactams, or carbapenems which form acyl-enzymes and adopt catalytically incompetent conformations that are poorly hydrolyzed. Irreversible “suicide inhibitors” can permanently inactivate the β-lactamase through secondary chemical reactions in the enzyme active site. Examples of irreversible suicide inactivators include the commercially available class A inhibitors clavulanic acid, sulbactam, and tazobactam.

Clavulanic acid, the first β-lactamase inhibitor introduced into clinical medicine, was isolated from Streptomyces clavuligerus in the 1970s, more than 3 decades ago. Clavulanate (the salt form of the acid in solution) showed little antimicrobial activity alone, but when combined with amoxicillin, clavulanate significantly lowered the amoxicillin MICs against S. aureus, K. pneumoniae, Proteus mirabilis, and E. coli. Sulbactam and tazobactam are penicillinate sulfones that were later developed by the pharmaceutical industry as synthetic compounds in 1978 and 1980, respectively. All three β-lactamase inhibitor compounds share structural similarity with penicillin; are effective against many susceptible organisms expressing class A β-lactamases (including CTX-M and the ESBL derivatives of TEM-1, TEM-2, and SHV-1); and are generally less effective against class B, C, and D β-lactamases. The activity of an inhibitor can be evaluated by the turnover number (tₙ) (also equivalent to the partition ratio [k_{cat}/k_{inact}]), defined as the number of inhibitor molecules that are hydrolyzed per unit time before one enzyme molecule is irreversibly inactivated. For example, S. aureus PC1 requires one clavulanate molecule to inactivate one β-lactamase enzyme, while TEM-1 needs 160 clavulanate molecules, SHV-1 requires 60, and B. cereus I requires more than 16,000. For comparison, sulbactam tₙs are 10,000 and 13,000 for TEM-1 and SHV-1, respectively.

The low K_{I}s of the inhibitors for class A β-lactamases (nM to μM), the ability to occupy the active site “longer” than β-lactams (high acylation and low deacylation rates), and the failure to be hydrolyzed efficiently are integral to their efficacy. Clavulanate, sulbactam, and tazobactam differ from β-lactam antibiotics as they possess a leaving group at position C-1 of the five-membered ring (sulbactam and tazobactam are sulfones, while clavulanate has an enol ether oxygen at this position). The better leaving group allows for secondary ring opening and β-lactamase enzyme modification. Compared to clavulanate, the unmodified sulfone in sulbactam is a relatively poor leaving group, a property reflected in the high partition ratios for this inhibitor (e.g., for TEM-1, sulbactam tₙ=10,000 and clavulanate tₙ=160). Tazobactam possesses a triazole group at the C-2 β-methyl position. This modification leads to tazobactam's improved IC₅₀s, partition ratios, and lowered MICs for representative class A and C β-lactamases.

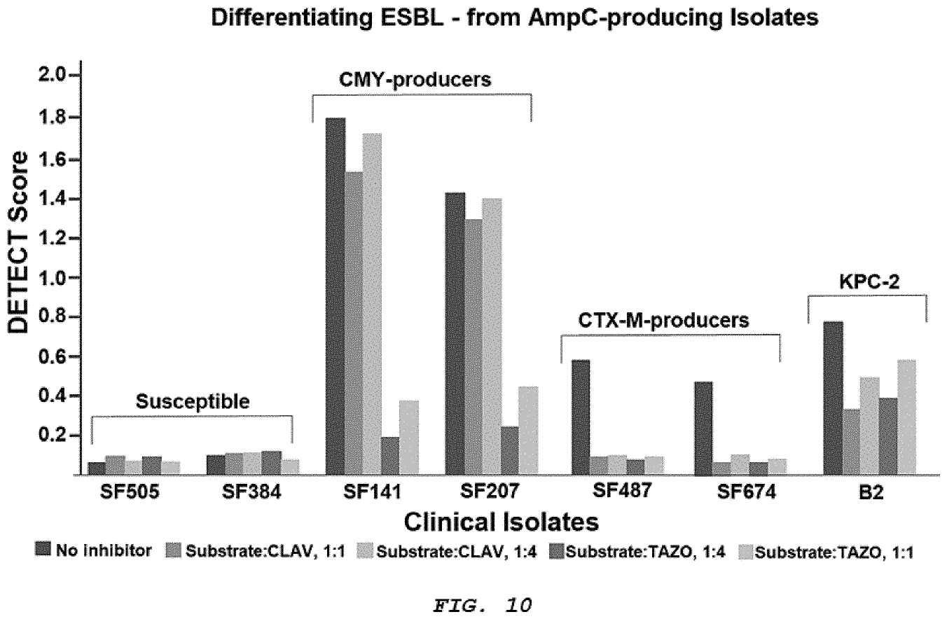

The efficacy of the mechanism-based inhibitors can vary within and between the classes of β-lactamases. For class A, SHV-1 is more resistant to inactivation by sulbactam than TEM-1 but more susceptible to inactivation by clavulanate. Comparative studies of TEM- and SHV-derived enzymes, including ESBLs, found that the IC₅₀s for clavulanate were 60- and 580-fold lower than those for sulbactam against TEM-1 and SHV-1, respectively. The explanations for these differences in inactivation chemistry are likely subtle, yet highly important, differences in the enzyme active sites. For example, atomic structure models of TEM-1 and SHV-1 indicated that the distance between Val216 and Arg244, residues responsible for positioning of the water molecule important in the inactivation mechanism of clavulanate, was more than 2 Å greater in SHV-1 than in TEM-1. This increased distance may be too great for coordination of a water molecule, suggesting that the strategic water is positioned elsewhere in SHV-1 and may be recruited into the active site with acylation of the substrate or inhibitor. This variation underscores the notion that mechanism-based inhibitors may undergo different inactivation chemistry even in highly similar enzymes. By using this difference in mechanism and susceptibility for β-lactamases, one can use the β-lactamase inhibitors in the methods disclosed herein to better identity target β-lactamases in a sample. For example, clavulanic acid was used in the methods disclosed herein to as a means to resolve CTX-M from CMY-producing GNB (e.g., see FIG. 10). As such, the disclosure fully recognizes that β-lactamases can be used in the methods of the disclosure in order to better identify one or more target β-lactamases in a sample.

The disclosure also provides for a kit which comprises one or more compounds disclosed herein. A kit will typically comprise one or more additional containers, each with one or more of various materials (such as reagents, optionally in concentrated form, and/or devices) desirable from a commercial and user standpoint for use of an oligosaccharide described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The disclosure further provides that the methods and compositions described herein can be further defined by the following aspects (aspects 1 to 54):

1. A compound having the structure of Formula I or Formula II:

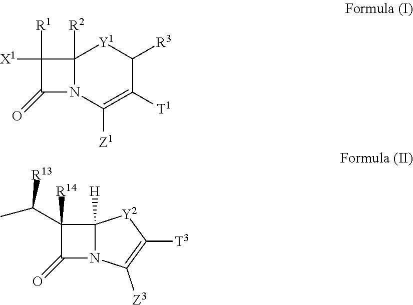

or a salt, stereoisomer, tautomer, polymorph, or solvate thereof, wherein:

T¹ is a benzenethiol containing group or Z², wherein if T¹ is Z², then Z¹ is T²;

Z¹ is a carboxylate, a carbonyl, an ester, an amide, a sulfone, a sulfonamide, a sulfonyl, —S(O)₂OH or T², wherein if Z¹ is T², then T¹ is Z²;

T² is a benzenethiol containing group;

T³ is a benzenethiol containing group

Z² is a carboxylate, a carbonyl, an ester, an amide, a sulfone, a sulfonamide, a sulfonyl, or —S(O)₂OH;

Z³ is a carboxylate, a carbonyl, an ester, an amide, a sulfone, a sulfonamide, a sulfonyl, or —S(O)₂OH;

X¹ is

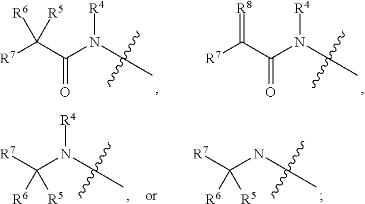

Y¹ is

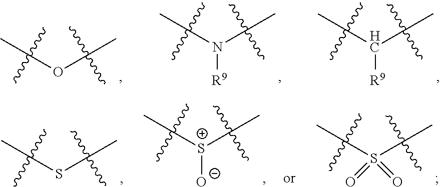

Y² is

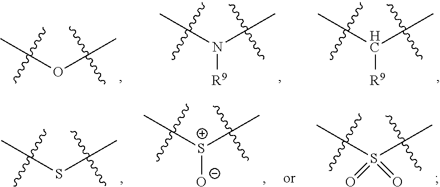

R¹-R⁶, R⁹-R¹¹, R¹³ and R¹⁴ are each independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle;

R⁷ is an optionally substituted (C₅-C₇) cycloalkyl, optionally substituted aryl, optionally substituted benzyl, or optionally substituted heterocycle; and

R⁸ is

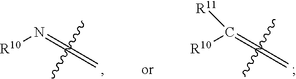

with the proviso that the compound does not have the structure of:

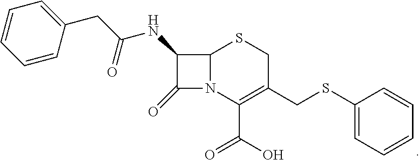 [SMILES: O=C(Cc1ccccc1)N[C@@H]1C(=O)N2C(C(=O)O)=C(CSc3ccccc3)CSC12]

2. The compound of aspect 1, wherein T¹ or T² is a benzenethiol group selected from the group consisting of:

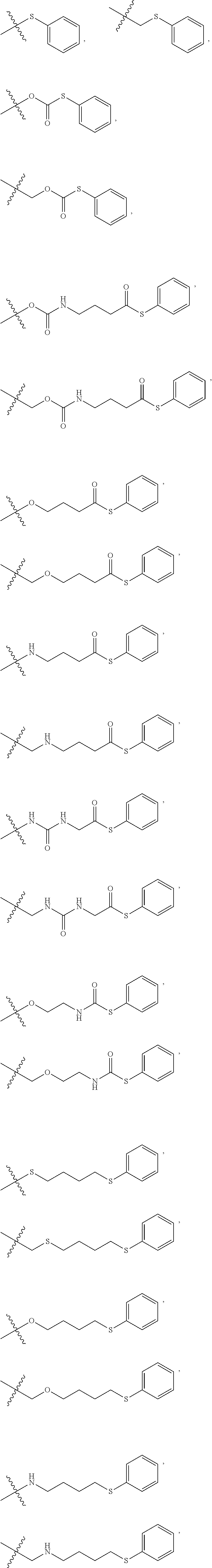

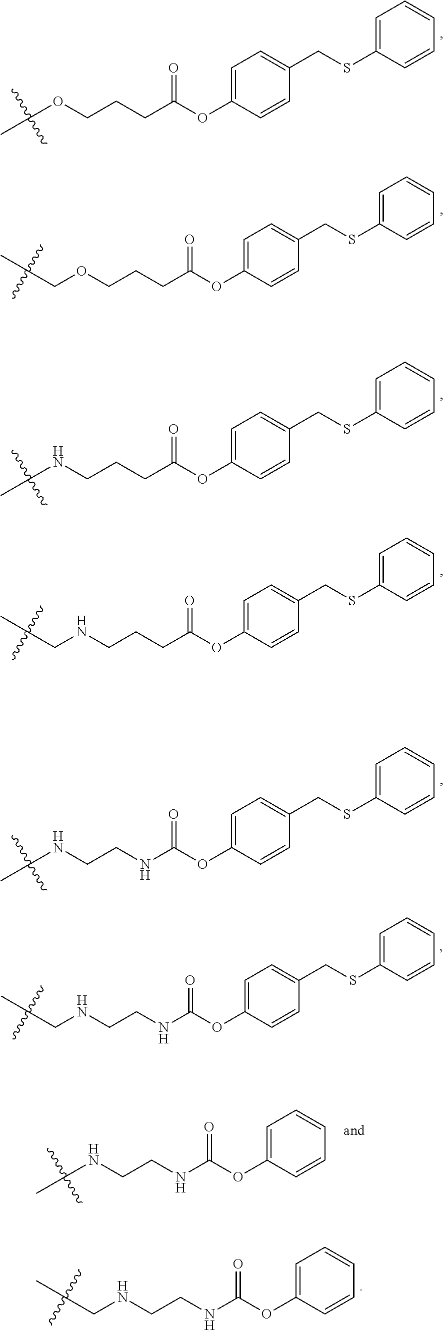

3. The compound of aspect 1 or aspect 2, wherein R⁷ is selected from the group consisting of:

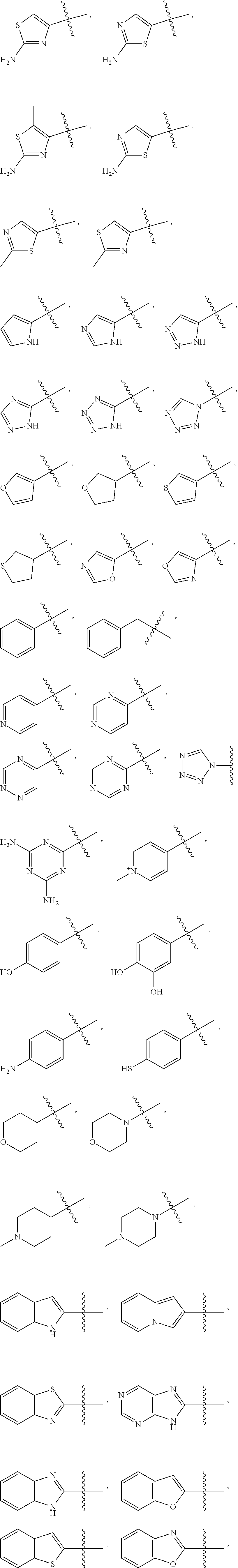

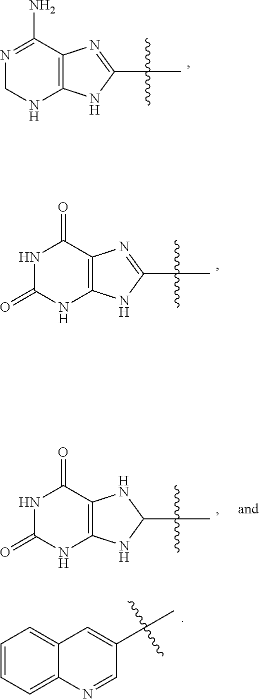 [SMILES: *c1nc2c([nH]1)NCN=C2N ; *c1nc2c(=O)[nH]c(=O)[nH]c2[nH]1 ; *C1Nc2[nH]c(=O)[nH]c(=O)c2N1 ; *c1cnc2ccccc2c1]

4. The compound of any one of the previous aspects, wherein the compound has a structure of Formula I(a):

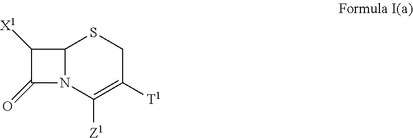

or a salt, stereoisomer, tautomer, polymorph, or solvate thereof, wherein:

T¹ is a benzenethiol containing group or Z², wherein if T¹ is Z², then Z¹ is T²;

Z¹ is a carboxylate, a carbonyl, an ester, an amide, a sulfone, a sulfonamide, a sulfonyl, —S(O)₂OH or T², wherein if Z¹ is T², then T¹ is Z²;

T² is a benzenethiol containing group;

Z² is a carboxylate, a carbonyl, an ester, an amide, a sulfone, a sulfonamide, a sulfonyl, or —S(O)₂OH;

X¹ is

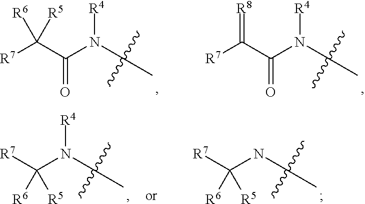

R⁴, R⁵, and R¹⁰ are independently an H or a (C₁-C₆)alkyl;

R⁶ is an H, or an amine;

R⁷ is an optionally substituted (C₅-C₇) cycloalkyl, optionally substituted aryl, optionally substituted benzyl, or optionally substituted heterocycle;

R⁸ is

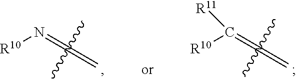

and

R⁹ is a hydroxyl or an (C₁-C₃)alkoxy.

5. The compound of aspect 4, wherein T¹ or T² is a benzenethiol group selected from the group consisting of:

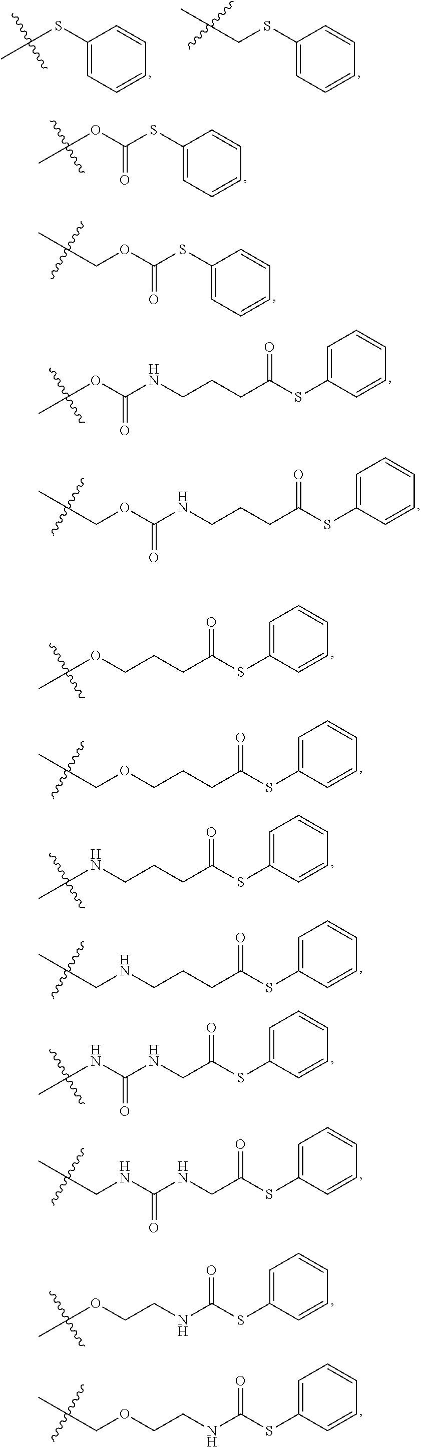

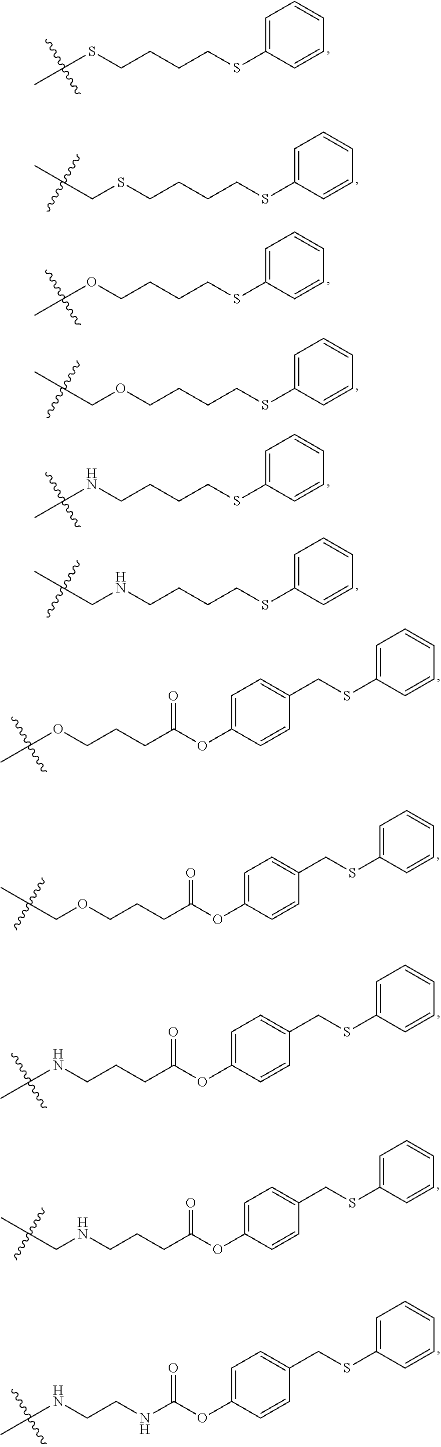

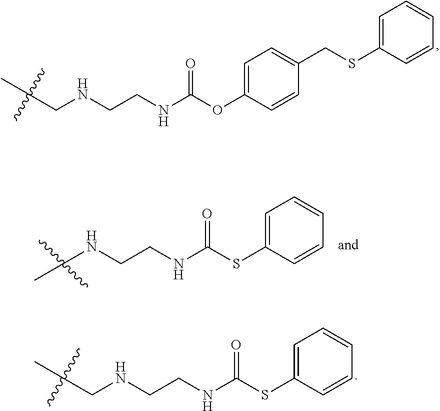 [SMILES: *CNCCNC(=O)Oc1ccc(CSc2ccccc2)cc1 ; *NCCNC(=O)Sc1ccccc1 ; *CNCCNC(=O)Sc1ccccc1]

6. The compound of aspect 4, wherein R⁷ is selected from the group consisting of:

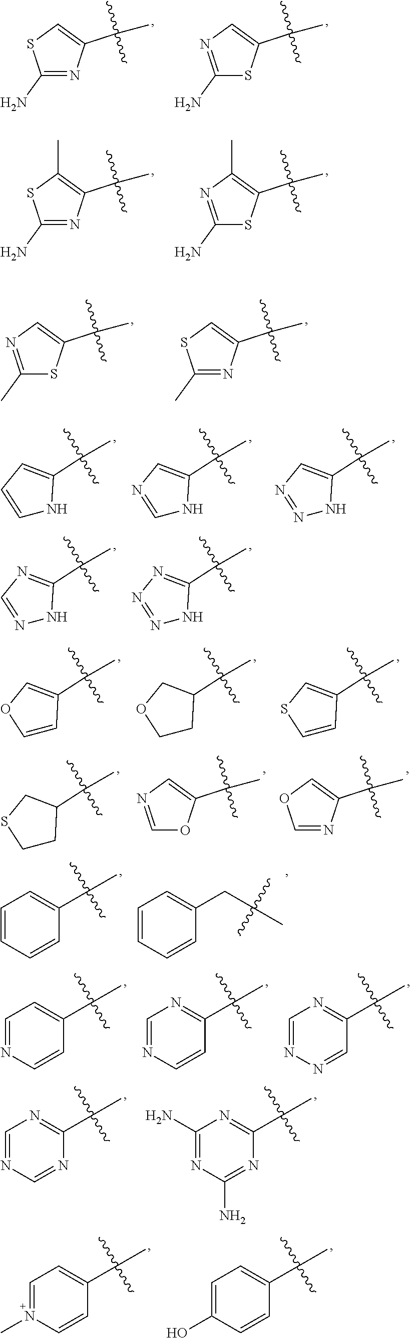

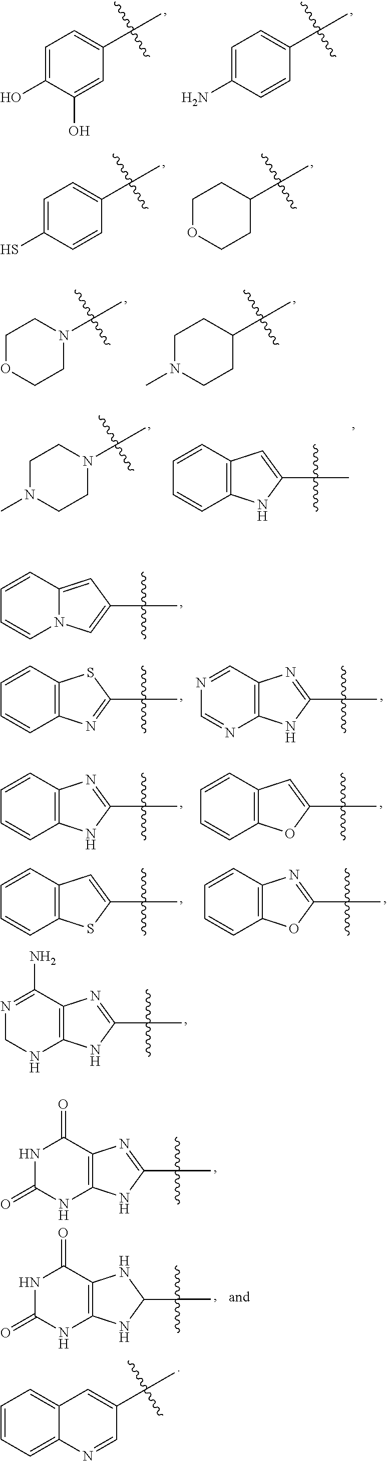

7. The compound of any one of the previous aspects, wherein the compound has the structure of Formula I(b):

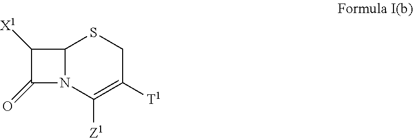

or a salt, stereoisomer, tautomer, polymorph, or solvate thereof, wherein:

T¹ a benzenethiol containing group selected from the group consisting

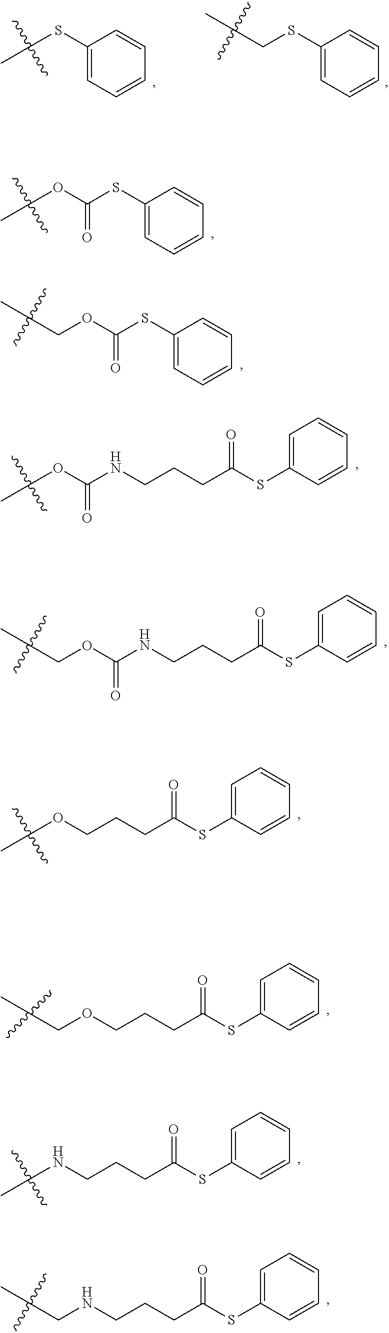

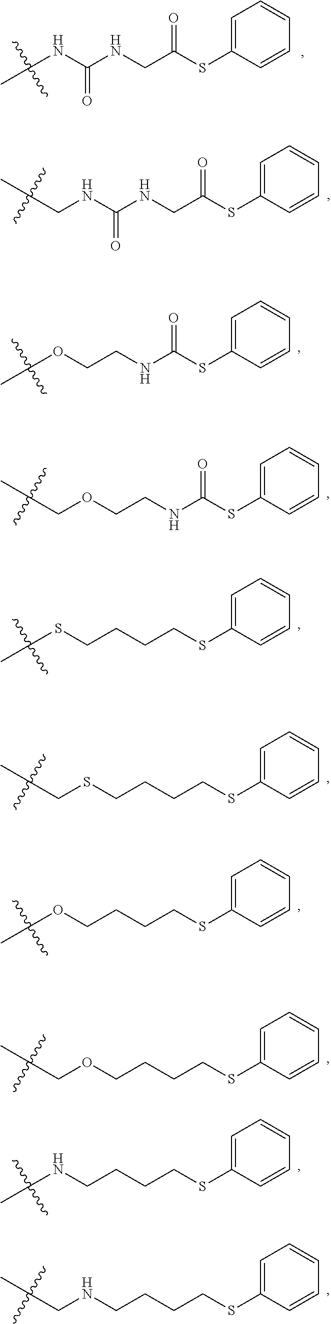

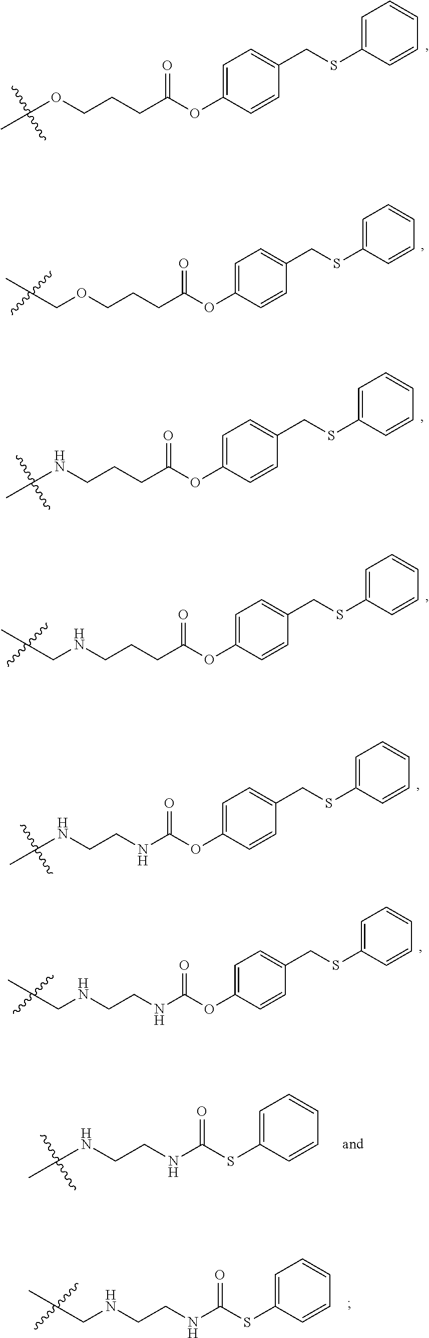

Z¹ is a carboxylate, a carbonyl, an ester, an amide, a sulfone, a sulfonamide, a sulfonyl, —S(O)₂OH or T²;

X¹ is

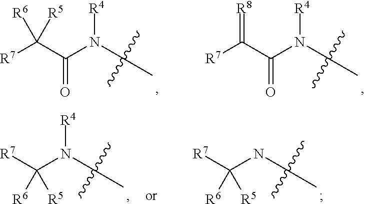

R⁴, R⁵, and R¹⁰ are independently an H or a (C₁-C₆)alkyl;

R⁶ is an H, or an amine;

R⁷ is an optionally substituted aryl, optionally substituted benzyl, or optionally substituted heterocycle;

R⁸ is

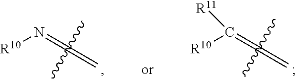

and

R⁹ is a hydroxyl or an (C₁-C₃)alkoxy.

8. The compound of aspect 7, wherein R⁷ is selected from the group consisting of:

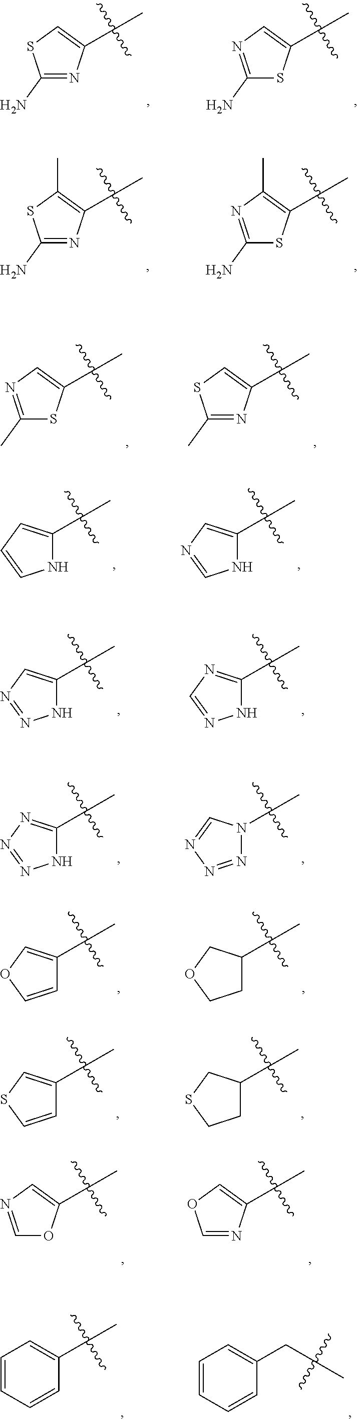

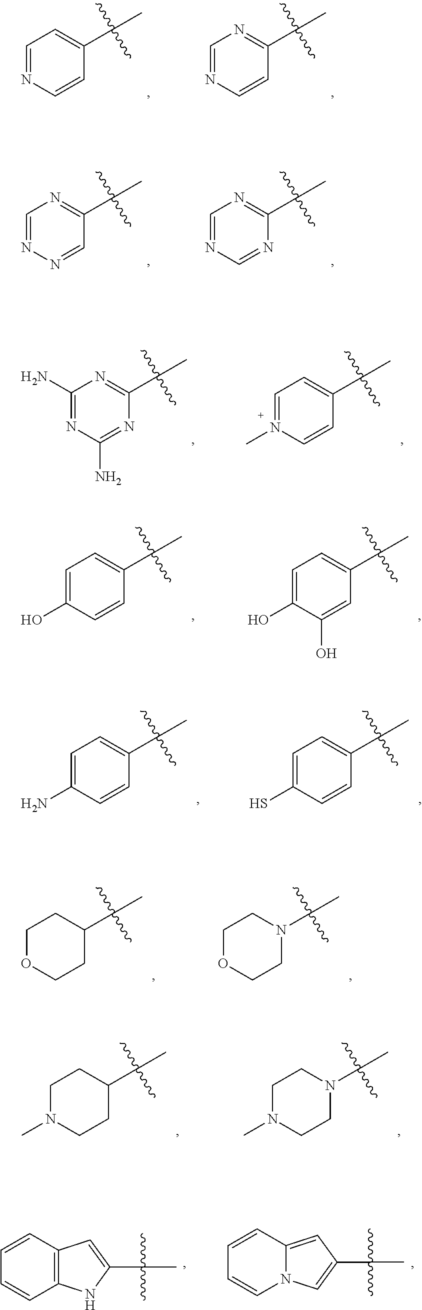

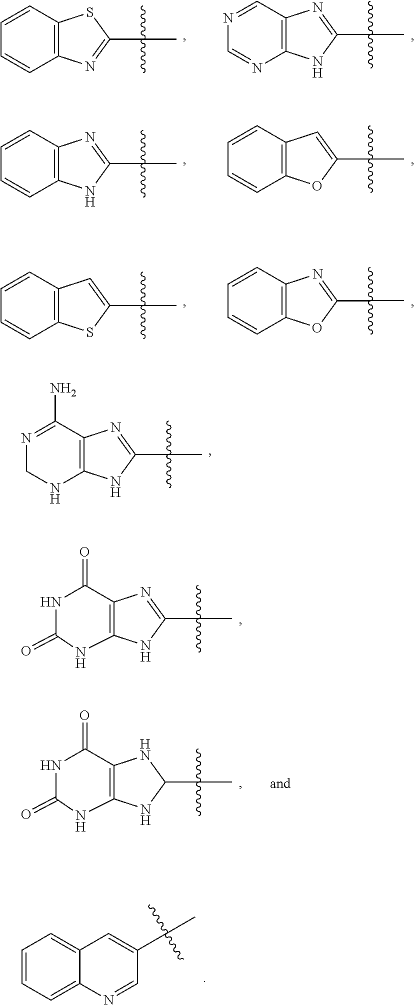

9. The compound of aspect 1, wherein the compound has the structure of Formula I(c):

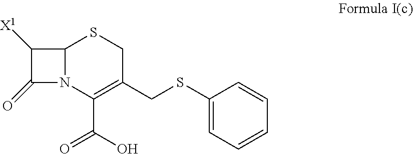

X¹ is

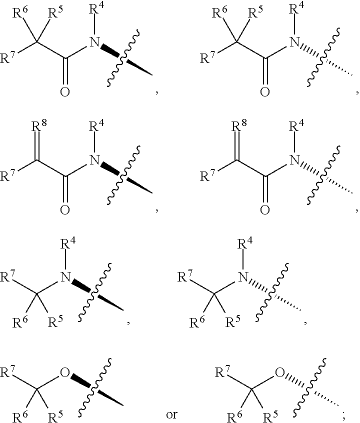

R⁴, R⁵, and R¹⁰ are independently an H or a (C₁-C₆)alkyl;

R⁶ is an H, or an amine;

R⁷ is selected from the group consisting of:

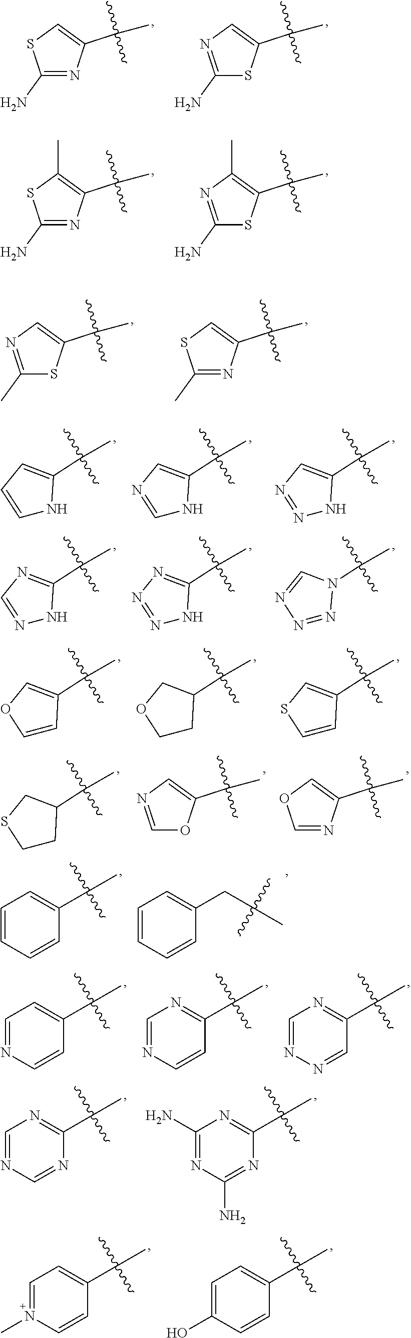

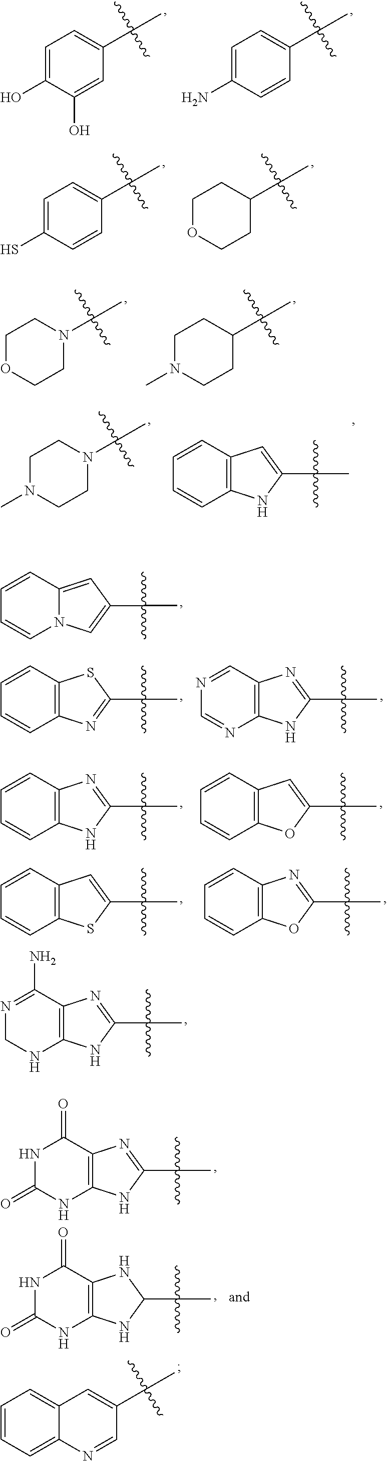

R⁸ is

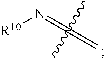

and

R⁹ is

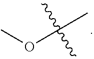 [SMILES: *OC]

10. The compound of any one of the previous aspects, wherein the compound is selected from the group consisting of:

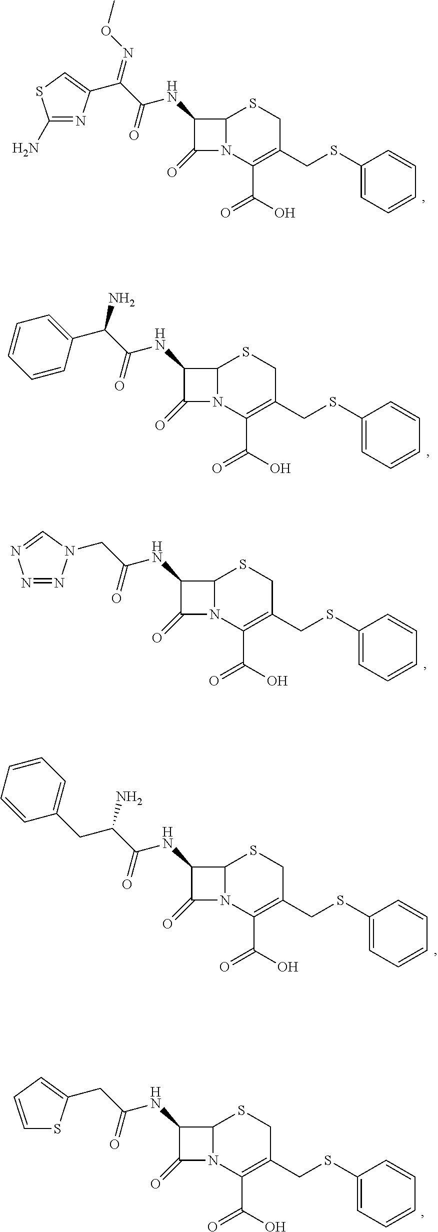 [SMILES: CO/N=C(/C(=O)N[C@@H]1C(=O)N2C(C(=O)O)=C(CSc3ccccc3)CSC12)c1csc(N)n1 ; N[C@@H](C(=O)N[C@@H]1C(=O)N2C(C(=O)O)=C(CSc3ccccc3)CSC12)c1ccccc1 ; O=C(Cn1cnnn1)N[C@@H]1C(=O)N2C(C(=O)O)=C(CSc3ccccc3)CSC12 ; N[C@@H](Cc1ccccc1)C(=O)N[C@@H]1C(=O)N2C(C(=O)O)=C(CSc3ccccc3)CSC12 ; O=C(Cc1cccs1)N[C@@H]1C(=O)N2C(C(=O)O)=C(CSc3ccccc3)CSC12]

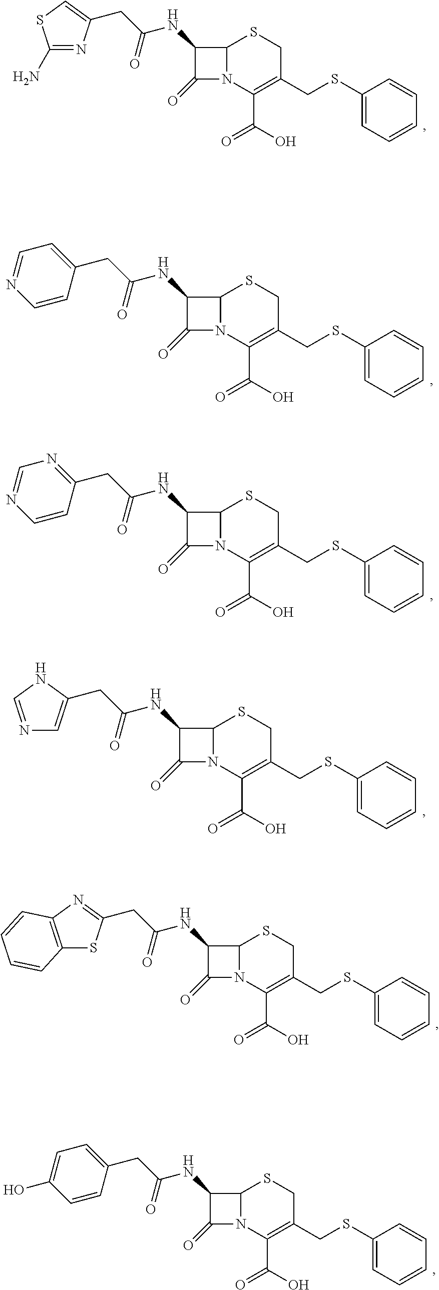 [SMILES: Nc1nc(CC(=O)N[C@@H]2C(=O)N3C(C(=O)O)=C(CSc4ccccc4)CSC23)cs1 ; O=C(Cc1ccncc1)N[C@@H]1C(=O)N2C(C(=O)O)=C(CSc3ccccc3)CSC12 ; O=C(Cc1ccncn1)N[C@@H]1C(=O)N2C(C(=O)O)=C(CSc3ccccc3)CSC12 ; O=C(Cc1cnc[nH]1)N[C@@H]1C(=O)N2C(C(=O)O)=C(CSc3ccccc3)CSC12 ; O=C(Cc1nc2ccccc2s1)N[C@@H]1C(=O)N2C(C(=O)O)=C(CSc3ccccc3)CSC12 ; O=C(Cc1ccc(O)cc1)N[C@@H]1C(=O)N2C(C(=O)O)=C(CSc3ccccc3)CSC12]

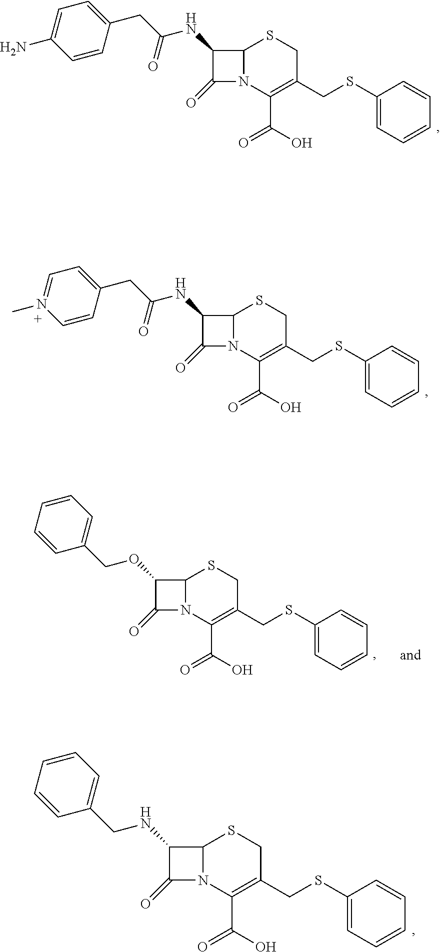

or a salt, stereoisomer, tautomer, polymorph, or solvate thereof.

11. The compound of aspect 10, wherein the compound has the structure of:

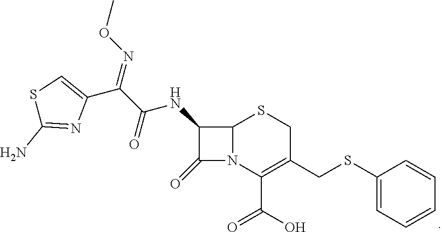 [SMILES: CO/N=C(/C(=O)N[C@@H]1C(=O)N2C(C(=O)O)=C(CSc3ccccc3)CSC12)c1csc(N)n1]

12. The compound of any one of the previous aspects, wherein T³ is a benzenethiol containing group selected from the group consisting of:

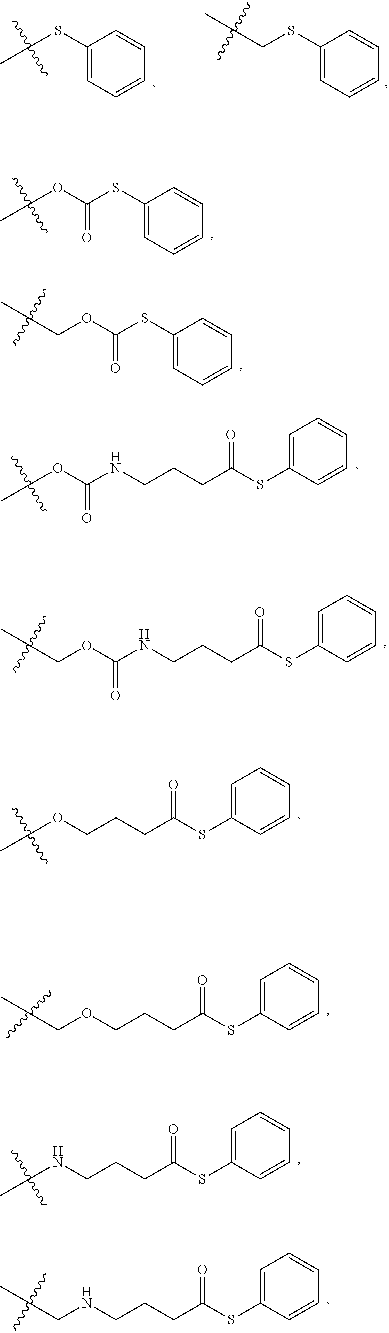

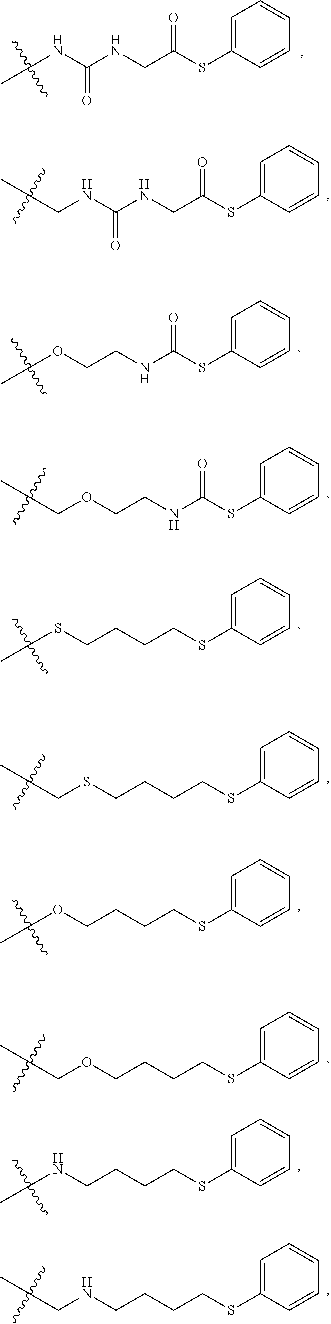

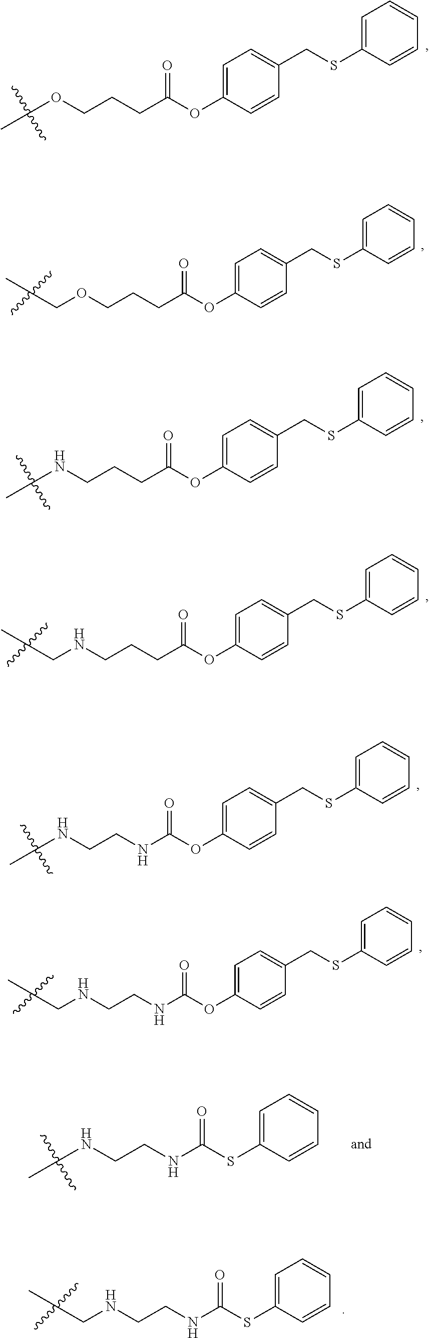

13. The compound of any one of the previous aspects, wherein the compound has the structure of Formula II(a):

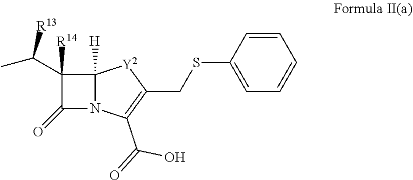

or a salt, stereoisomer, tautomer, polymorph, or solvate thereof, wherein:

Y2 is

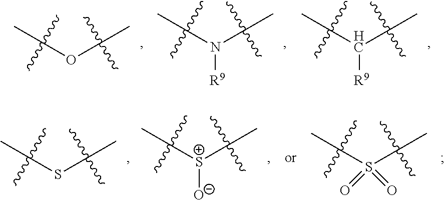

R⁹, R¹³ and R¹⁴ are independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle.

14. The compound of any one of the previous aspects, wherein the compound has the structure of Formula II(b):

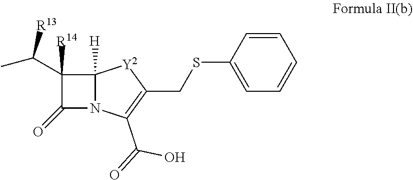

or a salt, stereoisomer, tautomer, polymorph, or solvate thereof, wherein:

Y² is

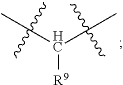

R⁹, R¹³ and R¹⁴ are independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, and optionally substituted (C₁-C₆)alkyl.

15. The compound of any one of the previous aspects, wherein the compound has a structure selected from:

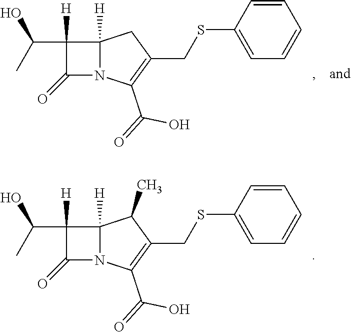 [SMILES: C[C@@H](O)[C@H]1C(=O)N2C(C(=O)O)=C(CSc3ccccc3)C[C@H]12 ; C[C@@H](O)[C@H]1C(=O)N2C(C(=O)O)=C(CSc3ccccc3)[C@H](C)[C@H]12]

16. The compound of any one of the previous aspects, wherein the compound is substantially a single enantiomer or a single diastereomer, wherein the compound has an (R) stereocenter.

17. A method to detect the presence of one or more target β-lactamases in a sample, comprising:

(1) adding reagents to a sample suspected of comprising one or more target β-lactamases, wherein the reagents comprise:
(i) a compound of any one of the preceding aspects;
(ii) a chromogenic substrate for a cysteine protease; and
(iii) a caged/inactive cysteine protease;
(iv) optionally, an inhibitor to specific type(s) or class(es) of β-lactamases;

(2) measuring the absorbance of the sample;

(3) incubating the sample for at least 10 min and then re-measuring the absorbance of the sample;

(4) calculating a score by subtracting the absorbance of the sample measured in step (2) from the absorbance of the sample measured in step (3);

(5) comparing the score with an experimentally determined threshold value; wherein if the score exceeds a threshold value indicates that the sample comprises the one or more target β-lactamases; and wherein if the score is lower than the threshold value indicates the sample does not comprise the one or more target β-lactamases.

18. The method of aspect 17, wherein for step (1), the sample is obtained from a subject.

19. The method of aspect 17 or 18, wherein the subject is a human patient that has or is suspected of having a bacterial infection.

20. The method of any one of aspects 17 to 19, wherein the human patient has or is suspected of having a urinary tract infection.

21. The method of any one of aspects 17 to 20, wherein for step (1), the sample is a blood sample, a urine sample, a cerebrospinal fluid sample, a saliva sample, a rectal sample, a urethral sample, or an ocular sample.

22. The method of aspect 21, wherein for step (1), the sample is a blood sample or urine sample.

23. The method of aspect 22, wherein for step (1), the sample is a urine sample.

24. The method of any one of aspects 17 to 22, wherein for step (1), the one or more target β-lactamases are selected from penicillinases, extended-spectrum β-lactamases (ESBLs), inhibitor-resistant β-lactamases, AmpC-type β-lactamases, and carbapenemases.

25. The method of aspect 24, wherein the ESBLs are selected from TEM β-lactamases, SHV β-lactamases, CTX-M β-lactamases, OXA β-lactamases, PER β-lactamases, VEB β-lactamases, GES β-lactamases, and IBC β-lactamase.

26. The method of aspect 24, where the one or more target β-lactamases comprise CTX-M β-lactamases.

27. The method of aspect 24, wherein the carbapenemases are selected from metallo-β-lactamases, KPC β-lactamases, Verona integron-encoded metallo-β-lactamases, oxacillinases, CMY β-lactamases, New Delhi metallo-β-lactamases, Serratia marcescens enzymes, IMIpenem-hydrolysing β-lactamases, NMC β-lactamases and CcrA β-lactamases.

28. The method of aspect 27, wherein the one or more target β-lactamases comprise CMY β-lactamases and/or KPC β-lactamases.

29. The method of aspect 28, wherein the one or more target β-lactamases further comprise CTX-M β-lactamases.

30. The method of any one of aspects 17 to 29, wherein for step (1)(ii), the chromogenic substrate for a cysteine protease is a chromogenic substrate for papain, bromelain, cathepsin K, calpain, caspase-1, galactosidase, seperase, adenain, pyroglutamyl-peptidase I, sortase A, hepatitis C virus peptidase, sindbis virus-type nsP2 peptidase, dipeptidyl-peptidase VI, deSI-1 peptidase, TEV protease, amidophosphoribosyl transferase precursor, gamma-glutamyl hydrolase, hedgehog protein, or dmpA aminopeptidase.

31. The method of aspect 30, wherein the chromogenic substrate for a cysteine protease is a chromogenic substrate for papain.

32. The method of aspect 31, wherein the chromogenic substrate for papain is selected from the group consisting of azocasein, L-pyroglutamyl-L-phenylalanyl-L-leucine-p-nitroanilide (PFLNA), Nα-benzoyl-L-arginine 4-nitroanilide hydrochloride (BAPA), pyroglutamyl-L-phenylalanyl-L-leucine-p-nitroanilide (Pyr-Phe-Leu-pNA), and Z-Phe-Arg-p-nitroanilide.

33. The method of aspect 31, wherein the chromogenic substrate for papain is BAPA.

34. The method of any one of aspects 17 to 33, wherein for step (1)(iii), the caged/inactive cysteine protease comprises a cysteine protease selected from the group consisting of papain, bromelain, cathepsin K, calpain, caspase-1, galactosidase, seperase, adenain, pyroglutamyl-peptidase I, sortase A, hepatitis C virus peptidase, sindbis virus-type nsP2 peptidase, dipeptidyl-peptidase VI, deSI-1 peptidase, TEV protease, amidophosphoribosyl transferase precursor, gamma-glutamyl hydrolase, hedgehog protein, and dmpA aminopeptidase.

35. The method of aspect 34, wherein the caged/inactive cysteine protease comprises papain.

36. The method of aspect 35, wherein the caged/inactive cysteine protease is papapin-S—SCH₃.

37. The method of any one of aspects 17 to 36, wherein for step (1)(iii), the caged/inactive cysteine protease can be re-activated by reaction with low molecular weight thiolate anions or inorganic sulfides.

38. The method of aspect 37, wherein the caged/inactive cysteine protease can be reactivated by reaction with a benzenethiolate anion.

39. The method of aspect 38, wherein the one or more target β-lactamases react with the compound of (i) to produce a benzenethiolate anion.

40. The method of aspect 39, wherein the benzenethiolate anion liberated from the compound of step (I1)(i) reacts with the caged/inactive cysteine protease to reactivate the cysteine protease.

41. The method of aspect 41, wherein the caged/inactive cysteine protease is papain-S—SCH₃.

42. The method of aspect 40, wherein the chromogenic substrate for a cysteine protease is BAPA.

43. The method of any one of aspects 17 to 42, wherein for step (2), the absorbance of the sample is measured at 0 min.

44. The method of any one of aspects 17 to 43, wherein for step (3), the sample is incubated for 15 min to 60 min.

45. The method of aspect 44, wherein the sample is incubated for 30 min.

46. The method of any one of aspects 17 to 45, wherein for steps (2) and (3), the absorbance of the sample is measured at a wavelength of 400 nm to 450 nm.

47. The method of aspect 46, wherein for steps (2) and (3), the absorbance of the sample is measured at a wavelength of 405 nm.

48. The method of any one of aspects 17 to 47, wherein for steps (2) and (3), the absorbance of the sample is measured using a spectrophotometer, or a plate reader.

49. The method of any one of aspects 17 to 48, wherein for step (5), the experimentally determined threshold value was determined by analysis of a receiver operating characteristic (ROC) curve generated from an isolate panel of bacteria that produce β-lactamases, wherein the one of more target β-lactamases have the lowest limit of detection (LOD) in the isolate panel.

50. The method of any one of aspects 17 to 49, wherein the method is performed with and without the inhibitor to specific type(s) or class(es) of β-lactamase in step (1)(iv).

51. The method of aspect 50, wherein a measured change in the score of step (4), between the method performed without the inhibitor and the method performed with the inhibitor indicates that the specific type or class of β-lactamases is present in the sample.

52. The method of aspect 50, wherein the inhibitor to specific type(s) or class(es) of β-lactamases is an inhibitor to class of β-lactamases selected from the group consisting of penicillinases, extended-spectrum β-lactamases (ESBLs), inhibitor-resistant β-lactamases, AmpC-type β-lactamases, and carbapenemases.

53. The method of aspect 52, wherein the inhibitor to a specific type(s) or class(es) of β-lactamases inhibits ESBLs but does not inhibit AmpC-type β-lactamases.

54. The method of aspect 53, wherein the inhibitor is clavulanic acid or sulbactam.

The following examples are intended to illustrate but not limit the disclosure. While they are typical of those that might be used, other procedures known to those skilled in the art may alternatively be used.

### EXAMPLES

Study Design. The DETECT assay was assessed for the ability to identify the activity of CTX-M β-lactamases/CTX-M-producing bacteria directly in urine samples from patients with suspected UTI. The DETECT system was tested across three levels of increasing complexity: first with purified recombinant β-lactamase enzymes, second with β-lactamase-producing clinical isolates, and third with clinical urine samples. The urine study was an IRB-approved clinical validation study utilizing urine samples from a local clinical laboratory of a county hospital that were undergoing routine urine culture, which mainly included urine samples from patients with suspected UTI. The urine study was blinded because urine sample positivity for a uropathogen and subsequent uropathogen identification, antimicrobial susceptibility, and β-lactamase-production were unknown to study investigators during the time of urine testing with DETECT and subsequent DETECT data analysis. All urine samples submitted to the clinical laboratory for urine culture during the study period were tested. No outliers were excluded.

Materials for DETECT reagents. All chemicals and solvents utilized were commercial grade unless otherwise indicated. L-cysteine hydrochloride, N-α-Benzoyl-L-arginine 4-nitroanilide hydrochloride (BAPA), S-Methyl methane-thiosulfonate (CAS 2949-92-0), and papain from caricapapaya (CAS 9001-73-4) were purchased from Sigma-Aldrich. Sodium acetate was purchased from Alfa Aesar. Glacial acetic acid was purchased from Fischer Scientific. Monobasic sodium phosphate was purchased from MP Bio. Dibasic sodium phosphate was purchased from Acros Organics. Sodium chloride was purchased from VWR Chemicals. BIS-TRIS and ethylenediamine tetraacetic acid were purchased from EMD Millipore. Thymol (CAS: 89-83-8) was purchased from Tokyo Chemical Inventory.

DETECT reagents. The DETECT system is composed of five main reagents: (1) buffer 1, a 50:50 sodium acetate:sodium phosphate buffer mixture (a sodium acetate solution prepared to 5 mM, pH 4.7, containing 50 mM NaCl and 0.5 mM EDTA, and a sodium phosphate solution prepared to 40 mM, pH 7.6, containing 2 mM EDTA), used to dissolve caged papain or to dilute recombinant enzymes and bacterial isolates; (2) buffer 2, a bis-Tris buffer (50 mM bis-Tris, pH 6.7, with 1 mM EDTA), used to dissolve BAPA; (3) β-lactamase probe, the targeting probe (thiophenol-β-lac), dissolved in acetonitrile (1 mg/800 μL unless otherwise indicated), with synthesis described in deBoer et al. 2018; (4) caged/inactivated papain (described below); and (5) BAPA (7.2 mg BAPA/2.5 mL “buffer 2” in 5% DMSO unless otherwise indicated).

Papain Caging. Ten mL of sodium acetate (50 mM, pH 4.5, containing 0.01% thymol) was transferred to a 25 mL round-bottom flask that was first rinsed with the buffer solution and was sparged with nitrogen gas. In a separate 100 mL round bottom flask, 29 mL of a phosphate buffer (20 mM, pH 6.7, 1 mM ETDA) was also subject to nitrogen saturation prior to being transferred into a 100 mL round-bottom flask containing a stir bar. After 15 min of degassing, the sodium acetate solution (1.5 mL) was transferred to a scintillation vial containing 79.9 mg of solid unmodified papain (0.003 mmol, 1 eq). The slurry was then transferred to the flask containing the phosphate buffer. A portion of the papain slurry solution was then transferred into a scintillation vial charged with 6 mg of L-cysteine hydrochloride (0.038 mmol, 13 eq) to dissolve the cysteine and to facilitate quantitative transfer of the cysteine into the reaction solution. The reaction flask was then left to stir in an ice bath (0° C.). After 15 min, S-methyl methanethiosulfonate (0.113 mmol, 33 eq) was pipetted directly into the reaction flask and the solution was left to stir under nitrogen. After 15 min, the reaction was removed from the ice bath and the final solution was transferred into dialysis tubing and dialyzed against a sodium acetate buffer solution to remove excess reagents. A total of three exchanges were performed prior to lyophilization of the final modified papain solution. A Nanodrop reading of each batch was taken to determine the concentration. The solution was then pipetted into 15 mL Falcon tubes, such that there would be 2.07 mg/mL of solution. The tubes were then frozen at −80° C. and lyophilized. The fully lyophilized solid was then subjected to quality control.

Recombinant β-lactamase expression and purification. The recombinant β-lactamases OXA-1, SHV-1, TEM-1, KPC-2, CMY-2, SHV-12, TEM-20, CTX-M-2, CTX-M-8, CTX-M-14, and CTX-M-15 were prepared and purified as described previously (deBoer et al. 2018). The concentration of each purified enzyme was determined by the NanoDrop (Thermo Fisher Scientific) Protein A280 method and the calculation presented in EQ 1.

C=A/(ε*b) (EQ. 1)

C is the molar concentration, A is the A_{280 nm}, ε is the molar extinction coefficient, and b is the path length in mm. The molar concentration was converted to μg/μL using the molecular weight of the recombinant enzyme. The molar extinction coefficients and the molecular weight of each recombinant β-lactamase are shown in TABLE 1, and were determined by submitting the amino acid sequence of the recombinant β-lactamases to the ProtParam tool on the Swiss Institute of Bioinformatics ExPASy resource portal (web.expasy.org/protparam/).

TABLE 1
Extinction coefficient and molecular weight of recombinant
enzymes.

| | Extinction | Molecular weight |
|---|---|---|
| r-β-lactamase | coefficient | (Da, g/mol) |
| OXA-1 | 42065 | 29328.22 |
| SHV-1 | 32095 | 30070.34 |
| TEM-1 | 28085 | 30103.31 |
| KPC-2 | 39545 | 30342.27 |
| CMY-2 | 93850 | 41050.97 |
| SHV-12 | 32095 | 30114.40 |
| TEM-20 | 28085 | 30103.25 |
| CTX-M-2 | 23950 | 29483.33 |
| CTX-M-8 | 25440 | 29235.00 |
| CTX-M-14 | 23950 | 29169.94 |
| CTX-M-15 | 23950 | 29304.18 |

Defining the limit of detection (LOD) for recombinant β-lactamase activity. The recombinant β-lactamases SHV-1, TEM-1, KPC-2, CMY-2, CTX-M-2, CTX-M-8, CTX-M-14, and CTX-M-15 were purified as described previously. The recombinant β-lactamases OXA-1, SHV-12, and TEM-20 were cloned and purified as described previously, with cloning primers designed in this study and described in TABLE 2. The detection limit for a given β-lactamase was determined by defining the lowest concentration at which DETECT could distinguish the signal output produced by a target β-lactamase from a negative control.

TABLE 2
Primers and information for β-lactamase gene cloning.

| | | Amplicon | Signal | Protein |
|---|---|---|---|---|
| Gene | Primer Sequence (5′ to 3′)^{ab} | size^{c} | sequence^{d} | length^{e} |
| OXA- | F: TATACATATGTCAACAGATATCTCTACTGTT | 773 bps | 25 aa | 260 aa |
| 1 | GCATCTCC (SEQ ID NO: 1) | | | |
| | R: GGTGCTCGAGTAAATTTAGTGTGTTTAGAA | | | |
| | TGGTGATCGCATTTTTC (SEQ ID NO: 2) | | | |
| SHV- | F: TATACATATGAGCCCGCAGCCGCTTG (SEQ | 815 bps | 21 aa | 274 aa |
| 12^{f} | ID NO: 3) | | | |
| | R: GGTGCTCGAGGCGTTGCCAGTGCTCGATCA | | | |
| | G (SEQ ID NO: 4) | | | |
| TEM- | F: TATACATATGCACCCAGAAACGCTGGTGAA | 809 bps | 23 aa | 272 aa |
| 20^{f} | AG (SEQ ID NO: 5) | | | |
| | R: GGTGCTCGAGCCAATGCTTAATCAGTGAGG | | | |
| | CACC (SEQ ID NO: 6) | | | |

bps, base pairs; aa, amino acids.
^{a}These primers are used with the cloning methods described previously.²
^{b}The underlined sequence in each primer represents nucleotides that bind the β-lactamase gene of interest during PCR.
^{c}The amplicon size expected after PCR; signal sequences are not amplified.
^{d}This signal sequence was not amplified during PCR. Signal sequences were not desired in the final recombinant protein.
^{e}The length of each recombinant protein includes an additional 9 aa due to addition of an ATG, cut site, and 6X-His tag to its sequence after insertion and expression from the pET26b+ vector.

Assay. A stock solution of each β-lactamase and four serial 2-fold dilutions were prepared (β-lactamases were quantified by NanoDrop). In a 96-well plate, 75 μL of caged papain solution and 75 μL of BAPA solution were transferred into 14 wells. To 10 of 14 wells, 4 μL of the five different β-lactamase concentrations were added to two test wells each. To two of the remaining wells, 4 μL of β-lactamase probe solution (“control 1” well) or 4 μL of stock β-lactamase solution (“control 2” well) were added. Then the last two control wells received 10 μL of a cysteine solution (0.0016 M) (“positive control” well). Finally, to each test well 4 μL of β-lactamase probe solution were added. The absorbance values at 405ₙₘ (A_{405 nm}) were recorded in 2 min intervals for 20 min with a microplate reader to define the time-dependent growth of the absorbance that corresponds to formation of the colorimetric p-nitroaniline product of DETECT. We defined 20 min as the endpoint for these experiments because the maximum absorbance values were not found to be greater at 30 min when testing recombinant β-lactamases.

Calculating LOD. Fourteen control samples were collected over these studies. We took the average of the final A_{405 nm} values for all control wells across all experiments, to normalize for potential batch variability. Control 1 conditions yielded the greater A_{405 nm} value of the two groups; therefore, our LOD threshold was defined as three-times the standard deviation of the average A_{405 nm} value of the control 1 dataset. The A_{405 nm} values were plotted against β-lactamase concentration for each tested β-lactamase, and a linear regression was performed. The final LOD concentration was extrapolated by defining x as the β-lactamase concentration.

Clinical isolates, and antimicrobial susceptibility testing (AST) for minimal inhibitory concentration (MIC). E. coli and K. pneumoniae clinical isolates tested with DETECT were obtained from samples of blood, urine, cerebrospinal fluid, and swabs (rectal, urethral, or ocular) from patients in hospitals or outpatient clinics in several locations: San Francisco General Hospital, USA (SF strains); Rio de Janeiro, Brazil (B, CB, D, FB, HAF, HCD, HON, and XB strains); Slo Paulo, Brazil; and University Health Services at the University of California Berkeley, USA (IT strains). Bacterial isolates were also obtained from the CDC and FDA Antibiotic Resistance Isolate Bank (CDC strains). Isolates were previously tested for susceptibility to β-lactams and for carriage of β-lactamase genes (cite above references). In addition, we performed broth microdilution testing with the β-lactams ampicillin, cephalexin, cefotaxime, and ceftazidime to obtain MICs. Broth microdilution testing with the β-lactams ampicillin, cephalexin, cefotaxime, and ceftazidime were performed in accordance with standards set by the Clinical and Laboratory Standards Institute (CLSI) to obtain minimal inhibitory concentrations (MICs).

DETECT with clinical isolates. Clinical isolates were subcultured from frozen glycerol stocks into Mueller-Hinton cation-adjusted broth (MHB), and shaken overnight at 37° C. for 16-20 h. To wash the cells, one mL of overnight broth culture was pelleted in a microfuge tube with a microcentrifuge, then the pellet was resuspended in one mL of “buffer 1.” The bacterial suspension was then prepared to an optical density at 600 nm (OD_{600 nm}) of 0.5 f 0.005 (where an OD_{600 nm} of 0.1=1.0×10⁸ CFU/mL). 5 μL of this whole-cell bacterial suspension was transferred to two wells of a 96-well plate, each well containing 75 μL of 0.6 mg/mL caged papain solution and 75 μL of 7.2 mg/2.5 mL BAPA solution. The incubation time was initiated when 4 μL of β-lactamase probe solution was added to one well (sample well) and 4 μL of acetonitrile was added to the second well (control well), where the second well was used as a control to evaluate non-specific background signal. At 0 min and 30 min of room temperature incubation, the A_{405 nm} values were collected with a microplate reader. The DETECT Score at 30 min was calculated with EQ. 2:

(A_{405 nm T30} sample well −A_{405 nm T30} control well)−(A_{405 nm T0} sample well −A_{405 nm T0} control well) (EQ. 2)

ROC curve analysis was performed to establish a positive threshold by which to assess individual DETECT Scores generated from clinical isolates. Recombinant β-lactamase results guided true positive and true negative designations for this analysis (for the 96-isolate panel): CTX-M and CMY-producing isolates were considered true positives (48 isolates), while all other isolates were considered true negatives (48 isolates). A clinical isolate generating a DETECT Score that was greater than the threshold value was considered positive by DETECT. The sensitivity and specificity of the DETECT assay were then determined.

bla expression analyses in clinical isolates. Procedures for RNA extraction, cDNA synthesis, and real-time quantitative reverse transcription PCR (qRT-PCR)—to assess expression of β-lactamase genes (bla genes)—were performed as described previously (deBoer el al., ChemBioChem 19:2173-2177 (2018)), with slight modifications. Isolates used in qRT-PCR analyses were subcultured from frozen glycerol stocks into MHB, and shaken overnight at 37° C. for 16-18 hours. To wash the cells, one mL of overnight broth culture was pelleted in a microfuge tube with a microcentrifuge, then the pellet was resuspended in one mL of fresh MHB. The bacterial suspension was then prepared to an OD_{600 nm} of 0.5-0.6 for use in RNA extractions. β-lactamase class-specific primers, or group-specific primers within a β-lactamase class, were utilized in qRT-PCR analyses to assess expression of different β-lactamase genes (bla genes) in clinical isolates. Primers were designed and validated in this study and are listed in TABLE 3.

TABLE 3
Primer sequences and other information for qRT-PCR

| bla | | | | Amplicon |
|---|---|---|---|---|
| gene(s) | Primer | Efficiency | Sequence 5′ → 3′ | (bps) |
| TEM | TEM-268 | 101.8% | F: GGTCGCCGCATACACTATTCT (SEQ ID NO: 7) | 159 |
| | | | R: TCCTCCGATCGTTGTCAGAAGT (SEQ ID NO: 8) | |
| SHV | SHV-68 | 100.7% | F: CGCAGCCGCTTGAGCAAATT (SEQ ID NO: 9) | 191 |
| | | | R: CTGTTCGTCACCGGCATCCA (SEQ ID NO: 10) | |
| CTX- | CTX1-681 | 97.5% | F: ACTGCCTGCTTCCTGGGTT (SEQ ID NO: 11) | 175 |
| M-g1 | | | R: TTTAGCCGCCGACGCTAATAC (SEQ ID NO: 12) | |
| CTX- | CTX9-681 | 101.3% | F: CTTACCGACGTCGTGGACTG (SEQ ID NO: 13) | 182 |
| M-g9 | | | R: CGATGATTCTCGCCGCTGAA (SEQ ID NO: 14) | |
| CMY | CMY-877 | 99.1% | F: TGGGAGATGCTGAACTGGCC (SEQ ID NO: 15) | 132 |
| | | | R: ATGCACCCATGAGGCTTTCAC (SEQ ID NO: 16) | |
| KPC | KPC-625 | 101.1% | F: TGGCTAAAGGGAAACACGACC (SEQ ID NO: 17) | 162 |
| | | | R: GTAGACGGCCAACACAATAGGT (SEQ ID | |
| | | | NO: 18) | |
| rpoB | rpoB | 103.3% | F: AAGGCGAATCCAGCTTGTTCAGC (SEQ ID | 148 |
| | expression | | NO: 19) | |
| | | | R: TGACGTTGCATGTTCGCACCCATCA (SEQ ID | |
| | | | NO :20) | |

Two biological replicate experiments were performed for expression analyses. To compare expression of the different bla genes across bacterial isolates, we assessed the level of expression of bla compared to the internal control rpoB within each strain, using EQ 3:

2^{-ΔC}^{_{T}}, where ΔC_{T}=C_{T-bla}−C_{T-rpoB} (EQ. 3)

DETECT with β-lactamase inhibitors. DETECT experiments incorporating the β-lactamase inhibitor, clavulanic acid, were performed in the same manner as described in “DETECT with clinical isolates”, except that a duplicate set of wells were also tested with clavulanate, at a ratio of 2:1 clavulanate:β-lactamase probe. A solution of sodium clavulanate was prepared to 1 mg/400 μL in “buffer 1”, and 4 μL of this solution was added to both the sample and control well for each isolate tested, two min prior to addition of β-lactamase probe or acetonitrile to the sample and control well, respectively. DETECT Scores generated from the original DETECT procedure were compared to DETECT Scores generated in the presence of clavulanic acid (procedures were performed simultaneously for each isolate); the times-change in DETECT Score was calculated with EQ. 4:

times −change=original DETECT score/inhibitor DETECT score (EQ. 4)

Clinical urine sample collection. Ethics approval for this study was provided by the Alameda Health System (AHS) IRB committee. Urine samples submitted to the Highland Hospital Clinical Laboratory from July 23 to July 27 and July 30 to August 4 were included in this study. Highland Hospital (Oakland, Calif.) is the largest hospital within AHS (236 inpatient beds), and its clinical laboratory provides microbiology services to two other hospitals and three wellness centers within the healthcare system. All urine samples submitted to the clinical laboratory for routine urine culture during the study period—which mainly represent urine from patients with suspected UTI—were utilized in this study. Urine samples were first used by clinical laboratory personnel for standard urine culture plating, then later (within the same day) used by study investigators. No clinical information was obtained from the patients whose urine samples were utilized in this study. Urine samples did not contain bacterial growth inhibitors/preservatives.

Urine culture, organism identification, AST, and ESBL confirmatory testing. Standard microbiological procedures were performed by the clinical laboratory as part of routine care for all urine samples used in this study, per the clinical laboratory's standard operating procedures. First, 1 μL or 10 μL of urine sample was plated on standard agar plates (blood agar and eosin methylene blue agar biplate), then visually inspected the next day for significant growth indicative of a UTI (≥10⁴ CFU/mL cutoff applied). The MiscroScan WalkAway system (Beckman Coulter) was utilized for bacterial identification and AST of GNB and select GPB causing UTI. The antimicrobial classes and agents tested were: β-lactams (ampicillin/sulbactam, aztreonam, cefazolin, cefepime, cefotaxime, cefoxitin, ceftazidime, ceftriaxone, ertapenem, imipenem, meropenem, and piperacillin/tazobactam), folate pathway inhibitors (trimethoprim/sulfamethoxazole), aminoglycosides (amikacin, gentamicin, and tobramycin), fluoroquinolones (ciprofloxacin and levofloxacin), nitrofurans (nitrofurantoin), and glycylcyclines (tigecycline). AST interpretations were based on CLSI's 2017 guidelines.

After the first step of standard urine plating was performed, the clinical laboratory would place the leftover urine samples in the refrigerator. That same day, study investigators would utilize the samples in this study. Prior to testing a urine sample with DETECT, urine samples were re-plated onto blood agar plates to enable CFU/mL estimates at the time of DETECT testing and to confirm that colony counts remained similar to those obtained by the clinical laboratory on initial plating. After overnight incubation at 37° C., uropathogens from these plates were subcultured to MHB and shaken overnight at 37° C. for 16-20 hours. The overnight broth cultures were prepared for frozen storage by mixing 1 mL of broth culture with 450 μL of sterile 50% glycerol in a cryovial, then the cryovials were stored at −80° C. To screen uropathogens for any β-lactam resistance, GNB (that lacked other β-lactam resistance previously tested for on the MicroScan) were tested for susceptibility to ampicillin using the standard disk-diffusion method according to CLSI. Additionally, uropathogens that tested resistant to a 3′-generation cephalosporin (cefotaxime, ceftriaxone, or ceftazidime on the MicroScan) were further tested with an ESBL-confirmatory test using the standard disk-diffusion method according to CLSI (with cefotaxime, cefotaxime/clavulanic acid, ceftazidime, and ceftazidime/clavulanic acid disks).

DETECT with urine samples, and urine sample characteristics. After urine samples were plated by the clinical laboratory, the leftover urine samples were placed in the refrigerator until study investigators arrived that same day to test the urine samples for this study. Urine samples were visually inspected, and appearance (color, clarity) was recorded. The pH of urine samples was also determined by aliquoting 1 mL of urine into a microfuge tube, then measuring the pH with a pH test strip by dipping the strip into the aliquoted urine and visually interpreting the results relative to the provided interpretation chart.

For DETECT testing, urine samples were swirled in a figure-eight pattern to mix, then 50 μL of urine was transferred to two wells of a 96-well plate, with each well containing 75 μL of 1.0 mg/mL caged papain solution and 75 μL of 6.4 mg/2.5 mL BAPA solution. The incubation time was initiated when 4 μL of β-lactamase probe solution was added to one well (sample well) and 4 μL of acetonitrile was added to the second well (control well), where the second well was used as a control to account for non-specific background signal from the urines. At 0 min and 30 min of room temperature incubation, an A_{405 nm} reading was collected with a microplate reader (Infinite M Nano, Tecan). The DETECT Score at 30 min was calculated.

To assess the performance of DETECT for the ability to identify CTX-M-producing bacteria in urine samples with uropathogen concentrations considered to be clinically relevant (≥10⁴ CFU/mL cutoff applied by the clinical laboratory), the following standard phenotypic and genotypic analyses were utilized as the reference test method: positive ESBL confirmatory test (phenotypic) and positive CTX-M sequencing result (genotypic). Therefore, urine samples containing clinically relevant concentrations of a GNB that yielded a positive ESBL confirmatory test result and was positive for carriage of bla_{CTX-M} were considered true positives by the reference test method, while all other samples were considered true negatives. The true positive (11 urine samples) and true negative (460 urine samples) designations were used to group urine DETECT Scores for ROC curve analysis, so that a positive threshold for DETECT could be established for interpretation of individual DETECT Scores. A urine sample generating a DETECT Score that was greater than the threshold value was considered positive by DETECT. The sensitivity and specificity of the DETECT assay were determined.

When possible, bacteria from urine samples generating discrepant DETECT results (false-positive or false-negative) were retested by DETECT as individual isolates, using the “DETECT with clinical isolates” procedure and positive threshold for interpretation of results.

DNA extraction, and PCR amplification of β-lactamase genes. All β-lactam-resistant GNB (resistant at least to ampicillin) were tested for carriage of bla_{TEM}, bla_{SHV}, and bla_{OXA} β-lactamase genes by PCR as described previously (deBoer et al. 2018), which includes testing for ESBL variants of TEM and SHV. Additionally, 3^{rd}-generation cephalosporin-resistant GNB were also tested for carriage of bla_{CTX-M} genes, and the AmpC genes bla_{CMY} and bla_{DHA}, by PCR as described previously (Tarlton 2018 and Dallenne). PCR amplicons were cleaned and sequenced by Sanger sequencing at the University of California, Berkeley DNA Sequencing Facility. Geneious® v.9.1.3 (Biomatters Ltd.) was used to visually inspect, edit, then align forward and reverse sequences to obtain a consensus sequence. Trimmed consensus sequences were aligned with known β-lactamase sequence variants—which were obtained from the database of K. Bush, T. Palzkill, and G. Jacoby (externalwebapps.lahey.org/studies/) and GenBank—to identify the β-lactamase variants present.

Statistical analysis. DETECT Scores generated from DETECT experiments with clinical isolates and urine samples were analyzed with a two-tailed t-test. Antimicrobial susceptibility categorical variables in CTX-M-producing or non-CTX-M-producing bacteria were analyzed with Fisher's exact test using GraphPad QuickCalcs software (www.graphpad.com/quickcalcs/catMenu/). ROC curve analysis was performed using Prism 8 (GraphPad Software). DETECT assay sensitivity and specificity were calculated with MedCalc (MedCalc Software, www.medcalc.org/calc/diagnostic_test.php). Positive and negative predictive values were also calculated with MedCalc. For all analyses, P<0.05 was considered statistically significant.

Preparation and Characterization of β-Lactamase Probes:

Scheme 1 presents a generalized scheme that can be used to make various β-lactamase probes of the disclosure.

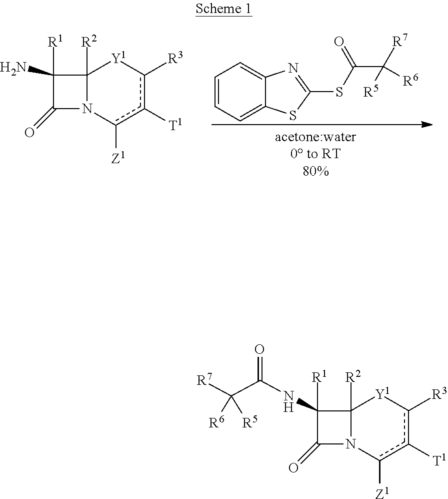

Scheme 2 provides for the production of (7R)-7-amino-8-oxo-3-((phenylthio)methyl)-5-thia-1-azabicyclo[4.2.0]oct-2-ene-2-carboxylic acid 4.

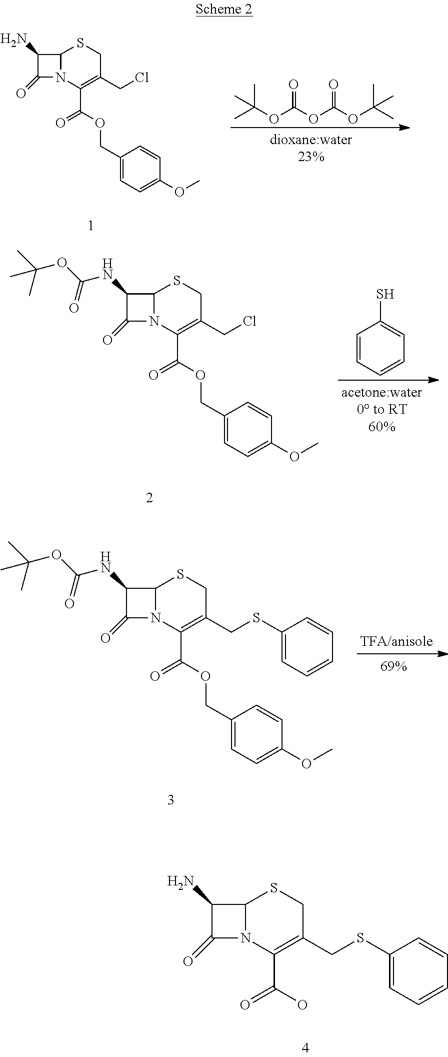

Scheme 3 provides the scheme used for the synthesis of Ceph-3 from 4, a representative example of a β-lactamase probe.

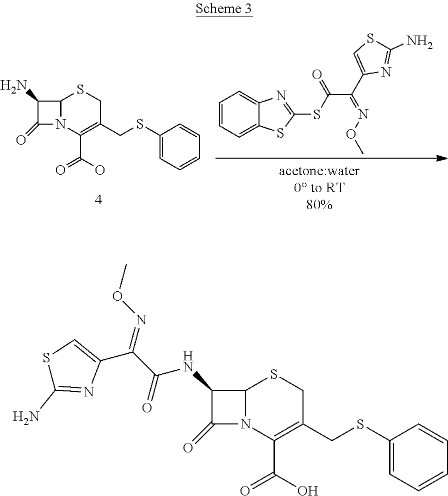

(7R)-7-((E)-2-(2-aminothiazol-4-yl)-2-(methoxyimino)acetamido)-8-oxo-3-((phenylthio)methyl)-5-thia-1-azabicyclo[4.2.0]oct-2-ene-2-carboxylic acid (Ceph-3):

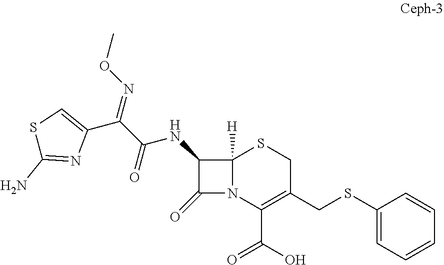 [SMILES: CO/N=C(/C(=O)N[C@@H]1C(=O)N2C(C(=O)O)=C(CSc3ccccc3)CS[C@H]12)c1csc(N)n1]

Triethylamine (18.2 μL, 0.131 mmol) was added to a solution on ice of (7R)-7-amino-8-oxo-3-((phenylthio)methyl)-5-thia-1-azabicyclo[4.2.0]oct-2-ene-2 carboxylic acid (20. mg, 0.62 mmol) in CH₂Cl₂ (4 mL). The resulting mixture was then allowed to warm to ambient temperature. To the mixture was added S-2-benzothiazolyl-2-amino-α-(methoxyimino)-4-thiazolethiolacetate (23.9 mg, 0.682 mmol). After the mixture was allowed to stir at ambient temperature for 5.5 h, the reaction was quenched with water. The organic layer was extracted with water (×5). The aqueous layers were combined and washed with CH₂Cl₂ (×3). The aqueous layer was then extracted with EtOAc (×4). The organic layers were combined, dried, and concentrated to afford the title compound as a pale-yellow powder. ¹H NMR (300 MHz, Acetone-d₆) δ 7.41 (m, J=32.5 Hz, 5H), 6.93 (s, 1H), 5.90 (s, 1H), 5.21 (s, 1H), 4.37 (s, 1H), 4.03 (s, 1H), 3.99-3.90 (m, 3H), 3.86 (s, 1H), 3.64 (s, 1H).

Scheme 4 presents a generalized scheme that can be used to make additional β-lactamase probes of the disclosure.

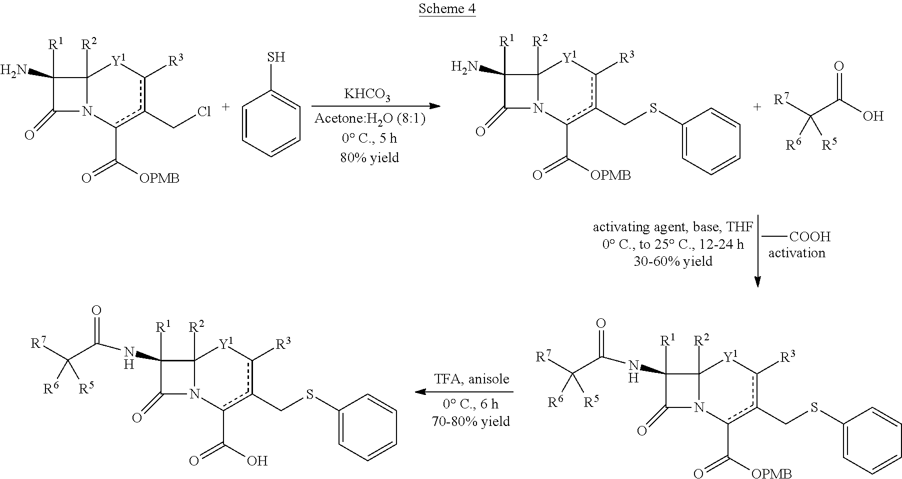

Scheme 5 provides a scheme that can be used to make Ceph-2-cephalexin 9.

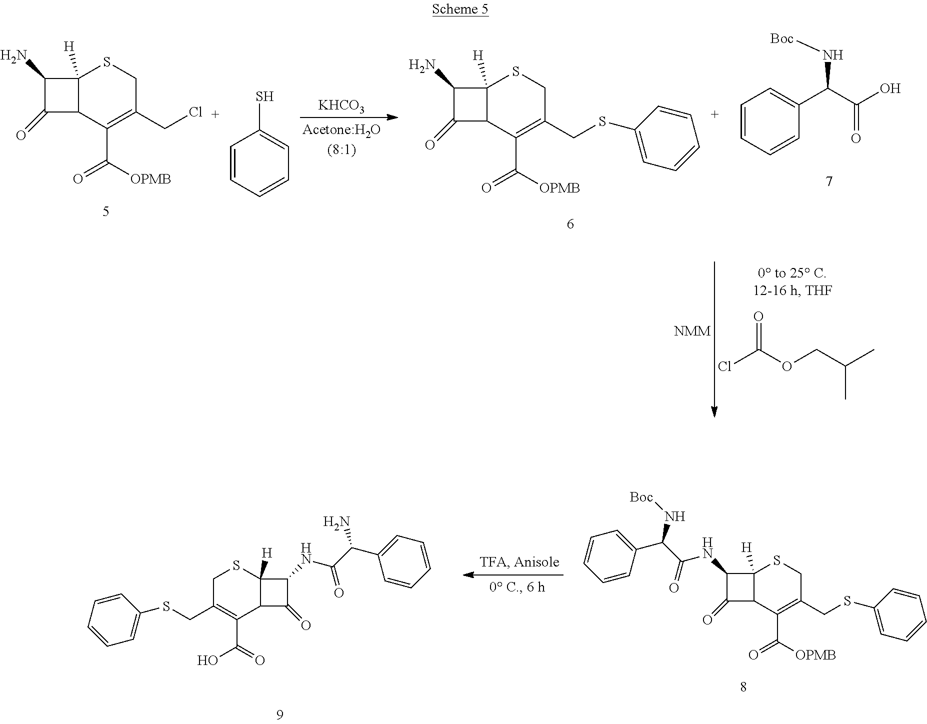

Step 1:

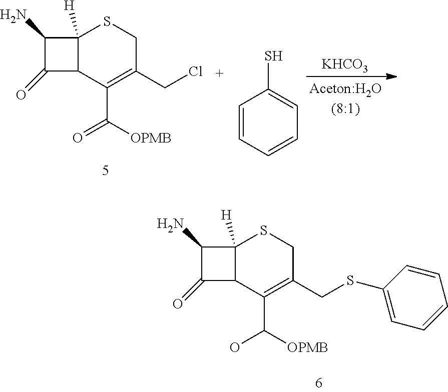

OPMB protected (1S,8R)-8-amino-7-oxo-4-((phenylthio)methyl)-2-thiabicyclo[4.2.0]oct-4-ene-5-carboxylic acid intermediate 6. In a 200-mL RBF, a slurry of chlorocephem 5 (1 g, 2.46 mmol) in acetone (79 mL) was prepared and stirred in an ice bath. A solution of KHCO₃ (0.40 g, 4 mmol) and thiophenol (0.41 mL, 4.018 mmol) was prepared in equal amounts of acetone and water (11 mL each) and allowed to stir for 5 min before adding dropwise to the reaction mixture. After adding all the thiophenol/KHCO3 solution to the mixture, the reaction was allowed to reach ambient temperatures and stirred for 6 h. The reaction mixture acidified to pH ˜0 using a pH 2 solution. To this acidified mixture, hexanes (25 mL) was added and allowed to stir for 5 min before separating the layers. The aqueous fraction was then washed two more times with hexanes and the aqueous layer was basified to pH >7 with concentrated KHCO₃ solution (˜25 mL). The basified aqueous layer was extracted with EtOAc (3×20 mL), and the combined organic was dried and concentrated to afford a yellow-orange solid (80% yield).

Step 2:

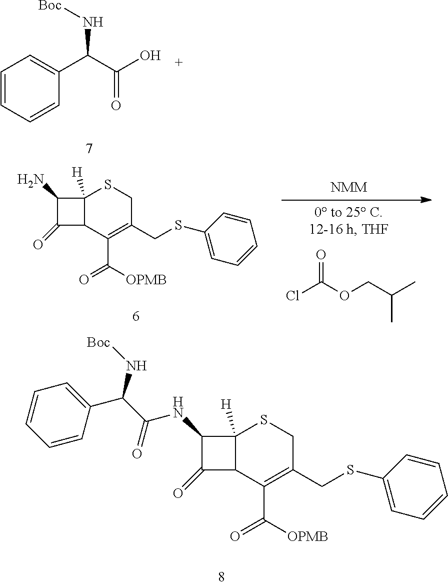

Boc and OPMB protected (1S,8R)-8-((R)-2-amino-2-phenylacetamido)-7-oxo-4-((phenylthio)methyl)-2-thiabicyclo[4.2.0]oct-4-ene-5-carboxylic acid intermediate 8. In a 25-mL RBF containing a solution of Boc-phenylglycine 7 (0.056 g, 0.226 mmol), N-methylmorpholine (25 μL, 0.226 mmol), and isobutyl chloroformate (29 μL, 0.226 mmol) in THF (4 mL) was stirred in an ice (0° C.) bath for 5 minutes to form the mixed anhydride intermediate under nitrogen. Meanwhile in a separate 25-mL flask, a solution of OPMB protected intermediate 6 (0.100 g, 0.226 mmol)) and N-methylmorpholine (NMM, 25 μL, 0.226 mmol) was prepared in THF (4 mL) and stirred on an ice bath. Under nitrogen, the intermediate mixture was slowly added to the mixed anhydride solution over the course of 5-7 minutes and the mixture stirred for 1 h at 0° C. After 1 h of stirring, the reaction mixture was returned to ambient temperatures and monitored by TLC (40/60, Hex/EtOAc) until majority of the OPMB protected intermediate 6 was consumed. R_{f} SM int.=0.40, R_{f} prominent prod spot=0.83, and R_{f} phenylglycine ˜0.50. After 12 h of reaction time, Ceph-2 intermediate was no longer observable by TLC. The reaction mixture was filtered to remove insoluble byproduct and the crude was concentrated to give a crude film solid on the sides of the flask. To this crude solid, 5-10 drops of THF was added and the flask was stored in 4° C. for 10 min. While swirling the flask, hexanes (10-15 mL) was added to crash out a white amorphous solid and the solid was filtered to collect. Any solid left behind the flask was re-dissolved with drops of THF and crashed out again with similar amounts of hexanes (10-15 mL) and filtered to collect solid product. The filtrate was analyzed by TLC to ensure that the soluble (colored usually) byproduct is removed and some product loss will be observed. The solid was collected in a vial and dried under high vacuum. The off-white amorphous solid had a weight of 0.069 g with 45% yield.

Step 3:

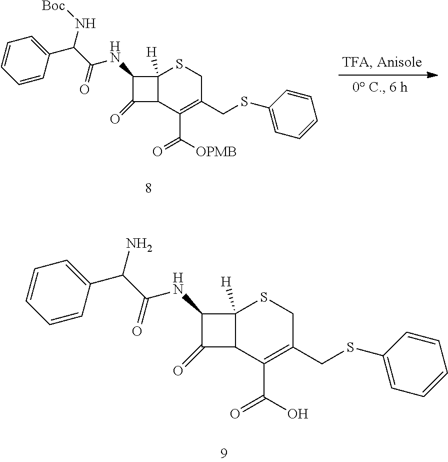

Ceph-2-cephalexin 9. A 8-mL vial BOC and OPMB protected intermediate 8 (0.034 g, 0.059 mmol) was charged with a stir bar and placed in an ice bath. In a separate vial, a mixture of TFA (160 μL) and anisole (160 μL) was prepared and this solution was slowly to the reaction vial. The reaction mixture stirred for 1 h at 0 C and allowed to reach ambient temperatures and stirred for another 4 h. After 5 h of stirring, an additional TFA (50 μL) and anisole (50 μL) mixture was added and allowed to stir for another hour. The reaction mixture was quenched with ethyl acetate (10 mL), and the organic layer was washed with brine until a neutral aqueous layer resulted. The organic layer was then dried with magnesium sulfate and concentrated to afford the crude compound containing residual anisole. The anisole was removed by adding excess hexanes (10 mL×3) and decanted several times. The product vial was placed under high vacuum to afford a pale orange solid (0.011 g).

DETECT preferentially identifies the activity of CTX-M β-lactamases. The selectivity of DETECT towards unique β-lactamases was studied by first defining the limit of detection (LOD) of a collection of purified recombinant β-lactamases. The recombinant enzymes tested represent common enzyme variants within major β-lactamase classes, and included: (a) OXA-1, a penicillinase; (b) TEM-1 and SHV-1, which are penicillinases/early-generation cephalosporinases; (c) major CTX-M variants, and TEM-20 and SHV-12, which are ESBLs; (d) CMY-2, an AmpC; and (e) KPC-2, a carbapenemase. These enzyme classes are found across diverse GNB, including the Enterobacteriaceae, Pseudomonas, and Acinetobacter.

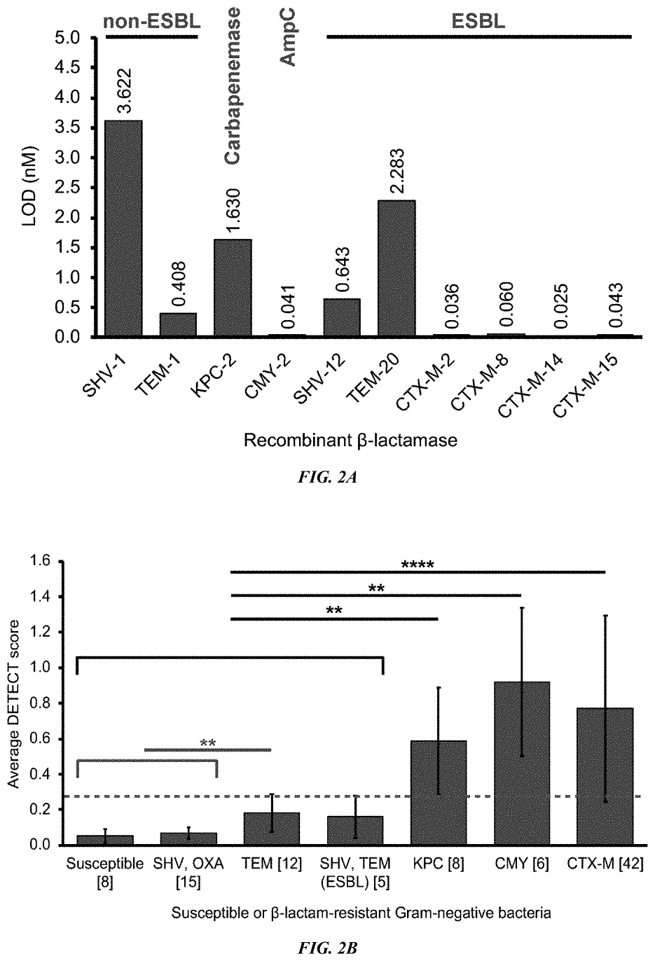

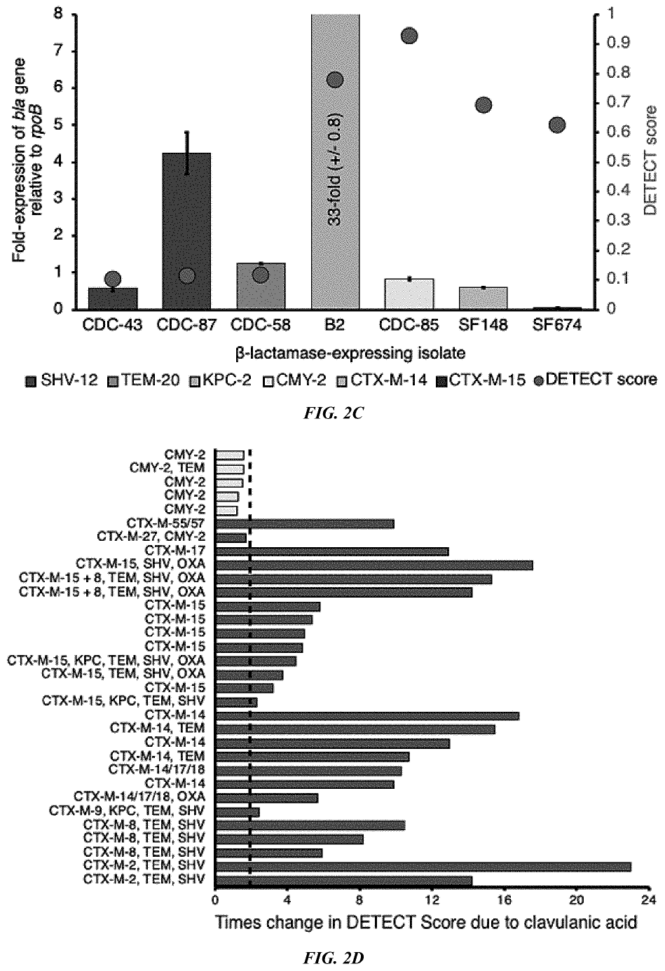

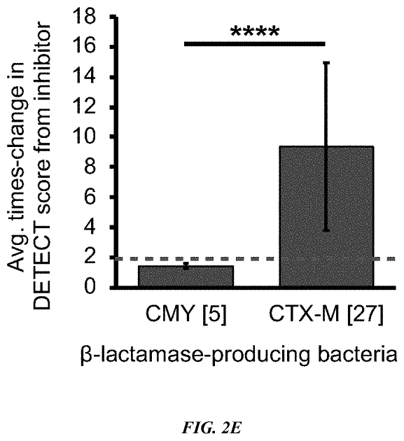

The LOD experiments demonstrated that the DETECT system (which currently utilizes a cephalosporin-like targeting probe) is highly sensitive to the enzymatic activity of the CTX-M β-lactamases, as well CMY (see FIG. 2A). The lowest LOD in DETECT was generated by CTX-M-14, with an LOD of 0.025 nM of purified recombinant enzyme. The other CTX-M variants tested—CTX-M-2, CTX-M-15, and CTX-M-8—as well as CMY-2, generated similarly low LODs of 0.036 nM, 0.043 nM, 0.060 nM, and 0.041 nM, respectively. The CTX-Ms and CMYs are similar in that they can mediate resistance to 3^{rd}-generation cephalosporins. Interestingly, the DETECT system was less sensitive to the enzymatic activity of other enzymes that mediate 3^{rd}-generation cephalosporin resistance, namely TEM and SHV ESBL variants and the KPC carbapenemase. At 2.3 nm, 1.6 nM, and 0.64 nM, the LODs of TEM-20, KPC-2, and SHV-12, respectively, were between 25 and 92 times higher than the LOD for CTX-M-14. The penicillinases/early-generation cephalosporinases SHV-1 and TEM-1 also generated higher LODs of 3.6 nm and 0.41 nM, which were 145 and 16 times greater, respectively, than the LOD for CTX-M-14. The OXA-1 penicillinase was very poor at activating the DETECT system; therefore, an approximate LOD was not obtained but was estimated to be at least greater than 4 μM.

DETECT can be applied to identify CTX-M-type β-lactamase activity in clinical isolates. While the enzymatic preference of CTX-M type β-lactamases towards a β-lactamase probe was demonstrated under biochemical conditions, clinical bacterial pathogens can be vastly diverse and complex. In particular, β-lactamase-producing uropathogens can produce a single or multiple β-lactamase variant(s) from a single bacterial strain. For example, TEM-1-producing E. coli isolated from one patient may produce significantly different levels of TEM-1 relative to a TEM-1 producing E. coli isolate cultured from another patient. Therefore, the capacity of DETECT to reveal the activity of CTX-M-type β-lactamases produced from clinical isolates was evaluated.

Experiments were performed to evaluate the capacity of DETECT to reveal the activity of CTX-M β-lactamases in bacterial isolates. In contrast to purified β-lactamase testing, clinical isolates represent a much more complex environment, where the same bacterial isolate may produce more than one type of β-lactamase, and where β-lactamase expression within and across bacterial isolates is variable.

A 96-isolate panel of roughly half clinical isolates of E. coli and half K. pneumoniae—the most common ESBL-producing GNB—were analyzed by DETECT. The isolates originated from multiple clinical sources and were previously characterized to produce a variety of β-lactamases, either singly or in combination (TABLE 4). These β-lactamases belonged to the same classes of enzymes previously tested in recombinant form, and included non-ESBL variants of TEM, SHV, and OXA; the CTX-M ESBLs, and ESBL variants of TEM and SHV; the plasmid-mediated AmpC (pAmpC) CMY; and the KPC carbapenemase. A full table of isolate characteristics—including β-lactamase content, select β-lactam minimal inhibitory concentrations (MICs), and DETECT Score—are shown in

TABLE 4
Clinical isolate panel tested with DETECT

| | | | | | Times-change |
|---|---|---|---|---|---|
| | | | List, all | DETECT | in DETECT |
| | Sample | | β-lactamases | score, | score, with |
| Isolate ID | Source | Organism | detected | 30 min | clavulanic acid |
| SF468 ♦ | Blood | E. coli | CTX-M-14, TEM-1 | 0.4795 | 15.5 |
| CDC-086 ♦ | unknown | E. coli | CTX-M-14, TEM-1B | 1.5331 | 10.7 |
| SF487 ♦ | Blood | E. coli | CTX-M-14 | 0.9356 | 9.9 |
| SF148 ♦ | Blood | E. coli | CTX-M-14 | 0.6913 | 16.8 |
| SF325 ♦ | Blood | E. coli | CTX-M-14/17/18, | 0.8829 | 5.7 |
| | | | OXA | | |
| SF473 ♦ | Blood | E. coli | CTX-M-14/17/18 | 0.8338 | 13.0 |
| D333 ♦ | Urine | E. coli | CTX-M-14/17/18 | 0.7205 | 10.3 |
| B7 ♦ | Blood | K. pneumoniae | KPC-2, CTX-M-15, | 0.7626 | 2.3 |
| | | | TEM-1B, SHV-11, | | |
| B23 ♦ | Blood | K. pneumoniae | KPC-2, CTX-M-15, | 0.2965 | 4.4 |
| | | | TEM-1B, SHV-11, | | |
| | | | OXA-1 | | |
| 160H | Urine | E. coli | CTX-M-15, OXA-1 | 1.1641 | |
| 56H | Blood | E. coli | CTX-M-15, OXA-1 | 1.1445 | |
| HCD405 ♦ | Rectal | K. pneumoniae | CTX-M-15, | 0.8921 | 17.6 |
| | swab | | SHV-25/121, OXA-1 | | |
| SF486 | Blood | E. coli | CTX-M-15, TEM-1B, | 0.0941 | |
| | | | OXA | | |
| CDC-109 | unknown | K. pneumoniae | CTX-M-15, TEM-1B, | 1.7614 | |
| | | | SHV-11, OXA-1 | | |
| SF681 ♦ | Blood | K. pneumoniae | CTX-M-15, TEM-1B, | 0.4004 | 3.8 |
| | | | SHV-11, OXA-1 | | |
| 164H | Urine | E. coli | CTX-M-15 | 1.2718 | |
| SF410 ♦ | Blood | E. coli | CTX-M-15 | 0.7971 | 4.8 |
| SF674 ♦ | Blood | E. coli | CTX-M-15 | 0.6239 | 5.8 |
| D497 ♦ | Urine | E. coli | CTX-M-15 | 0.3917 | 3.2 |
| D362 ♦ | Urine | E. coli | CTX-M-15 | 0.3022 | 4.9 |
| D14 ♦ | Urine | E. coli | CTX-M-15 | 0.2359 | 5.4 |
| D159 ♦ | Urine | E. coli | CTX-M-15 | 0.1275 | |
| FB13 ♦ | Blood | K. pneumoniae | CTX-M-15, CTX-M-8, | 1.0845 | 15.3 |
| | | | TEM-1A, SHV-25/121, | | |
| | | | OXA-1 , | | |
| FB90 | Blood | K. pneumoniae | CTX-M-15, CTX-M-8, | 0.5558 | 14.2 |
| | | | TEM-1A, SHV-25/121, | | |
| | | | OXA-1 | | |
| CDC-044 | unknown | K. pneumoniae | CTX-M-15, SHV-12, | 0.8077 | |
| | | | TEM-1A, OXA-9, | | |
| | | | OXA-1 | | |
| D270 ♦ | Urine | E. coli | CTX-M-17 | 0.5809 | 12.9 |
| D129 ♦ | Urine | E. coli | CTX-M-2, TEM, | 0.3692 | 14.2 |
| | | | SHV | | |
| 169H | Blood | E. coli | CTX-M-2 | 2.1705 | |
| 44H | Urine | E. coli | CTX-M-2 | 1.9969 | |
| HON257 ♦ | Rectal | K. pneumoniae | CTX-M-2, TEM-15, | 0.9368 | 23.0 |
| | swab | | SHV-25/121 | | |
| HON187 | Rectal | K. pneumoniae | CTX-M-2, TEM-15, | 0.1570 | |
| | swab | | SHV-25/121 | | |
| D500 ♦ | Urine | E. coli | CTX-M-27, | 0.7527 | 1.7 |
| | | | CMY-2/130 | | |
| 24H | Urine | E. coli | CTX-M-27, TEM-1 | 0.1287 | |
| D304 ♦ | Urine | E. coli | CTX-M-55/57 | 0.5546 | 9.9 |
| HCD309 ♦ | Rectal | K. pneumoniae | CTX-M-8, TEM-1, | 0.1890 | 5.9 |
| | swab | | SHV-1 | | |
| HAF102 ♦ | Rectal | K. pneumoniae | CTX-M-8, TEM-1, | 0.4589 | 8.2 |
| | swab | | SHV-76 | | |
| HAF66 | Rectal | K. pneumoniae | CTX-M-8, TEM-1, | 0.5852 | 10.5 |
| | swab | | SHV-85 | | |
| 64H | Urine | E. coli | CTX-M-8, TEM-1B, | 1.4513 | |
| | | | OXA-1 | | |
| 122H | Urine | E. coli | CTX-M-8 | 1.5232 | |
| HCD140 | Rectal | K. pneumoniae | CTX-M-8, SHV-27, | 1.2486 | |
| | swab | | TEM-1 | | |
| B14 ♦ | Blood | K. pneumoniae | KPC-2, CTX-M-9, | 0.3525 | 2.4 |
| | | | TEM-1A, SHV-11 | | |
| HON109 | Blood | K. pneumoniae | CTX-M-9/51, | 0.0710 | |
| | | | SHV-9/129 | | |
| CDC-012 | unknown | K. pneumoniae | SHV-12 | 0.3744 | |
| CDC-087 | unknown | K. pneumoniae | SHV-12 | 0.1128 | |
| CDC-043 | unknown | K. pneumoniae | SHV-12 | 0.1016 | |
| ATCC | Urine | K. pneumoniae | SHV-18 | 0.1039 | |
| 700603 | | | | | |
| CDC-058 | unknown | E. coli | TEM-20 | 0.1147 | |
| CDC-081 ♦ | unknown | E. coli | CMY-2, TEM-1B | 0.3660 | 1.6 |
| SF141 ♦ | Blood | E. coli | CMY-2 | 1.3759 | 1.5 |
| SF207 ♦ | Blood | E. coli | CMY-2 | 1.2087 | 1.2 |
| CDC-085 ♦ | unknown | E. coli | CMY-2 | 0.9272 | 1.3 |
| CDC-089 ♦ | unknown | E. coli | CMY-2 | 0.4563 | 1.6 |
| CDC-010 | unknown | K. pneumoniae | CMY-94, SHV-1 | 1.1873 | |
| B1 | Rectal | K. pneumoniae | KPC-2, SHV-11 | 0.6883 | |
| | swab | | | | |
| B3 | Rectal | K. pneumoniae | KPC-2, SHV-11 | 0.6446 | |
| | swab | | | | |
| B28 | Rectal | K. pneumoniae | KPC-2, SHV-11 | 0.2485 | |
| | swab | | | | |
| B21 | Urine | K. pneumoniae | KPC-2, SHV-11, | 0.2550 | |
| | | | OXA-1 | | |
| B2 | Rectal | E. coli | KPC-2 | 0.7773 | |
| | swab | | | | |
| CDC-061 | unknown | E. coli | KPC-3, TEM-1A, | 0.6584 | |
| | | | OXA-9 | | |
| CDC-112 | unknown | K. pneumoniae | KPC-3 | 1.1109 | |
| CDC-104 | unknown | E. coli | KPC-4, TEM-1A | 0.3092 | |
| SF310 | Blood | E. coli | OXA | 0.0795 | |
| IT115 | Urine | E. coli | OXA-1 | 0.0098 | |
| HCD422 | Urine | K. pneumoniae | SHV-1 | 0.1024 | |
| IT1335 | Urine | E. coli | SHV-1 | 0.0932 | |
| XB27 | Blood | K. pneumoniae | SHV-1 | 0.0829 | |
| IT30 | Urine | E. coli | SHV-1 | 0.0644 | |
| IT527 | Urine | E. coli | SHV-1 | 0.0035 | |
| HCD23 | Ocular | K. pneumoniae | SHV-11 | 0.0899 | |
| | swab | | | | |
| CB27 | Blood | K. pneumoniae | SHV-11 | 0.0867 | |
| CB52 | Blood | K. pneumoniae | SHV-132 | 0.0806 | |
| FB1 | Blood | K. pneumoniae | SHV-185 | 0.0957 | |
| FB45 | Blood | K. pneumoniae | SHV-38/168 | 0.0866 | |
| XB50 | Blood | K. pneumoniae | SHV-62 | 0.0622 | |
| HCD435 | blood | K. pneumoniae | SHV-83 | 0.0646 | |
| HON313 | Blood | K. pneumoniae | SHV-83/187 | 0.0312 | |
| SF176 | Blood | E. coli | TEM | 0.3386 | |
| IT2495 | Urine | E. coli | TEM-1A | 0.1939 | |
| IT11 | Urine | E. coli | TEM-1A | 0.1343 | |
| HON70 | Urethral | K. pneumoniae | TEM-1A, SHV-75, | 0.2646 | |
| | swab | | OXA-1 | | |
| SF105 | Blood | E. coli | TEM-1B | 0.3579 | |
| SF334 | Blood | E. coli | TEM-1B | 0.2551 | |
| IT372 | Urine | E. coli | TEM-1B | 0.1133 | |
| IT1173 | Urine | E. coli | TEM-1B | 0.0751 | |
| IT1158 | Urine | E. coli | TEM-1B, OXA-1 | 0.146 | |
| IT2532 | Urine | E. coli | TEM-1C | 0.0931 | |
| IT1004 | Urine | E. coli | TEM-1C | 0.0272 | |
| HCD120 | Rectal | K. pneumoniae | TEM, SHV | 0.1891 | |
| | swab | | | | |
| SF634 | Blood | K. pneumoniae | None detected | 0.1104 | |
| SF519 | Blood | K. pneumoniae | None detected | 0.0886 | |
| SF384 | Blood | E. coli | None detected | 0.0814 | |
| SF505 | Blood | E. coli | None detected | 0.0583 | |
| IT917 | Urine | E. coli | None detected | 0.0426 | |
| SF412 | Blood | K. pneumoniae | None detected | 0.0414 | |
| IT370 | Urine | E. coli | None detected | 0.0006 | |
| IT905 | Urine | E. coli | None detected | 0.0000 | |

* The chromosomal AmpC of E. coli was not screened for by PCR, and of the K. pneumoniae chromosomal β-lactamases, only SHV was properly screened for.
♦ Isolates labelled with this symbol were used in DETECT experiments incorporating clavulanic acid. Times-change in DETECT score was determined, comparing scores from the original DETECT assay to those from the DETECT + inhibitor assay (original score/inhibitor score).

DETECT Scores generated from isolates were grouped based on β-lactamase content in the cells (see FIG. 2B). Since more than one-third of the isolates produced multiple β-lactamases (a common feature in clinical isolates), a rank order was established to guide appropriate group placement for analyses, and was as follows: CTX-M >CMY >KPC >ESBL SHV or ESBL TEM >TEM >SHV or OXA >β-lactam-susceptible. Hence, CMY-containing isolates were grouped together regardless of other β-lactamase content (unless the isolate contained a CTX-M, in which case it was grouped with other CTX-Ms), and so forth.

In alignment with recombinant β-lactamase results, the CTX-M-producing and CMY-producing isolates were preferentially identified by the DETECT system, generating the highest average DETECT Scores at 30 min in comparison to other isolates (see FIG. 2B). The average DETECT Score of CTX-M-producing isolates was 0.77—roughly 4 to 15 times greater than the average Scores for SHV/TEM ESBL, TEM, SHV or OXA, and β-lactam-susceptible isolates (P<0.0001 for all). Similarly, the average DETECT Score of CMY-producing isolates was 0.92—roughly 5 to 18 times greater than the average Scores for the four other groups (P<0.01 for all). Interestingly, KPC-producing isolates also generated higher DETECT Scores, with an average Score of 0.59, which was between 3 and 12 times greater than the average Scores for the four non-CTX-M and non-CMY groups (P<0.01 for all). A ROC curve was generated to establish a threshold value for a positive DETECT Score. Recombinant β-lactamase results guided true positive and true negative groupings for the ROC curve; namely, CTX-M and CMY-producing isolates were considered true positives (48 isolates), while all other isolates were considered non-targets (48 isolates). This resulted in an AUC of 0.895 (95% CI: 0.832 to 0.958). A threshold value of 0.2806 was selected to optimize high sensitivity (85%) and specificity (81%). Apart from several of the KPC-producing isolates, false-positive results were generated by two TEM-1-producing E. coli and one SHV-12 (ESBL)-producing K. pneumoniae.

Expression analyses on an abbreviated panel of single β-lactamase-producing isolates were performed to investigate the higher-than-expected DETECT Scores from KPC-producing isolates (see FIG. 2C). qRT-PCR for bla genes and the internal control rpoB demonstrated that bla_{KPC-2} expression in the carbapenem-resistant E. coli isolate “B2” (with high DETECT Score, 0.8) was 33-fold higher than expression of rpoB. In comparison, the isolate with the next highest β-lactamase expression was “CDC-87” (with low DETECT Score, 0.1), an SHV-12 ESBL-producing isolate with 4-fold higher expression of bla_{SHV-12} compared to rpoB. While both isolates would be predicted to generate low DETECT Scores based on purified enzyme experiments, the high DETECT Score from the KPC-producing isolate may be attributed to relatively high levels of KPC compared to other β-lactamases, if expression patterns indeed reflect quantity of protein in the cells.

The possibility of differentiating between CMY (AmpC) and CTX-M (ESBL)-producing isolates was explored through the incorporation of the β-lactamase inhibitor, clavulanic acid, into DETECT. Clavulanic acid is a known inhibitor of ESBLs, but does not appreciably inhibit the activity of AmpC enzymes. A subset of the E. coli and K. pneumoniae clinical isolates were tested simultaneously with the original DETECT system and the DETECT-plus-inhibitor system, revealing that all isolates generated lower DETECT Scores at 30 min when clavulanic acid was added to the system. However, the extent to which the DETECT Score was affected (the times-change in Score) was associated with the type of β-lactamase produced (see FIG. 2D). The times-change in DETECT Score (original DETECT Score divided by inhibitor DETECT Score) was lower in CMY-producing isolates compared to CTX-M-producing isolates, as CMY is less susceptible to the inhibitor. A times-change threshold was generated to demarcate changes in DETECT Score indicative of a non-CMY/non-AmpC β-lactamase, and was determined to be 1.97×. The times-change in Score from all isolates containing CMY was under this threshold (including a dual CMY and CTX-M containing isolate), while the times-change in score from all other isolates containing CTX-M was above this threshold, indicating the ability to differentiate between these β-lactamase-producing isolates when needed.

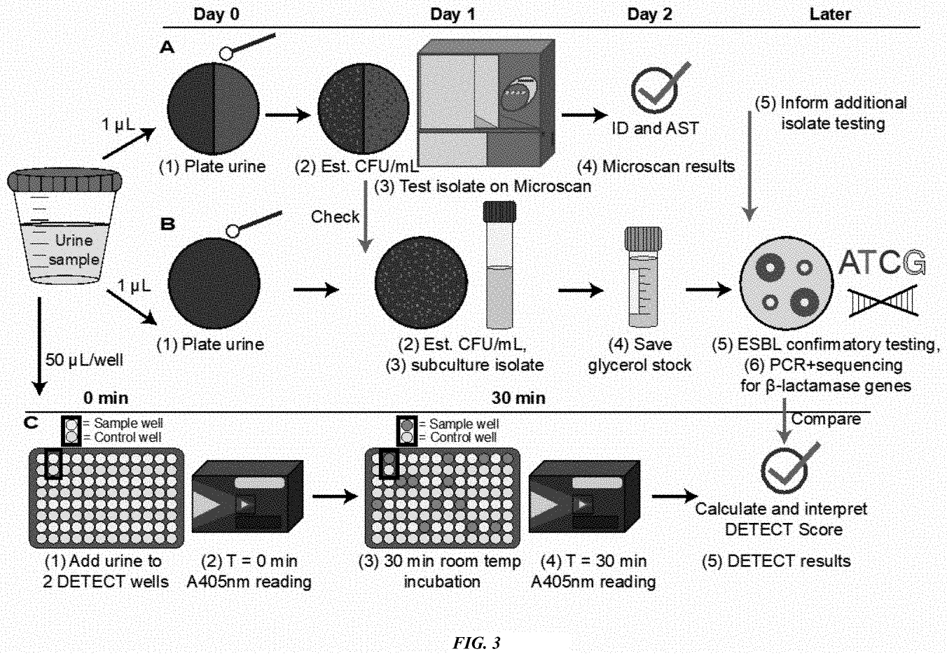

DETECT identifies CTX-M-producing bacteria in unprocessed urine samples. The clinical potential of DETECT as a diagnostic test was evaluated in unprocessed clinical urine samples to detect the presence of CTX-Ms as an indicator of ESBL-UTIs. The complex and diverse milieu of clinical urine samples represents one technological hurdle that impedes the use of biochemical-based approaches for direct detection of β-lactamase activity in urine. Accordingly, an RB-approved study at a public hospital in Oakland, Calif., was performed where all urine samples submitted to the clinical laboratory for urine culture over an 11-day period were tested. The DETECT assay was performed on urine samples without applying sample feature exclusions such as defined sample collection methods; pH, color, or clarity restrictions; CFU/mL cutoffs; or pathogen identification inclusion criteria. The workflow for this clinical urine study is illustrated in FIG. 3, including standard microbiological procedures performed by the clinical laboratory as part of routine testing (see FIG. 3A), microbiology and molecular biology procedures performed by study investigators (see FIG. 3B), and the DETECT assay, performed by study investigators (see FIG. 3C). The DETECT assay is rapid; after the addition of a small volume of unprocessed urine sample (100 μL in total) to the DETECT reagents, the test is complete in 30 min.

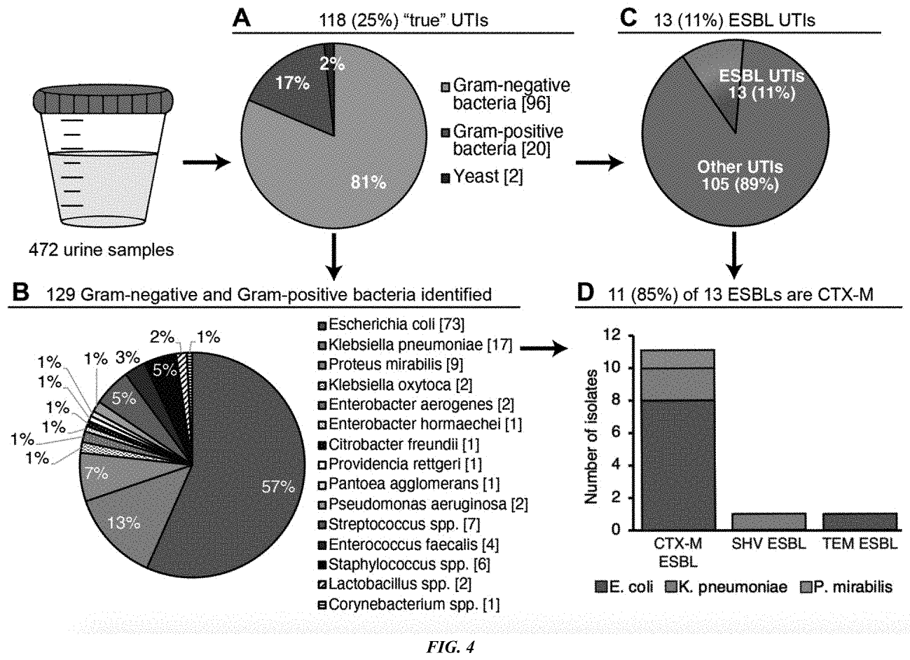

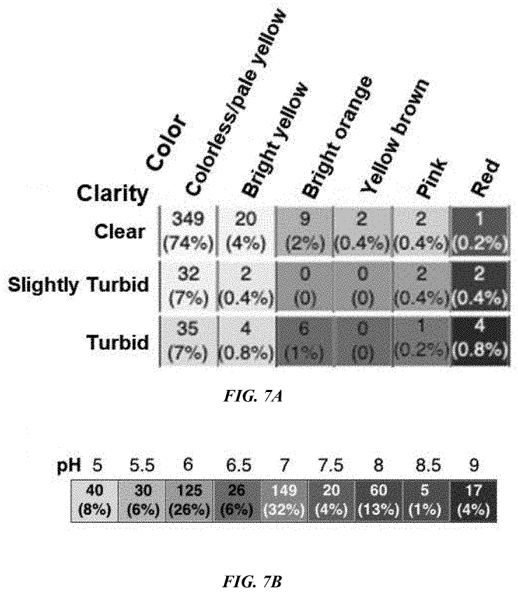

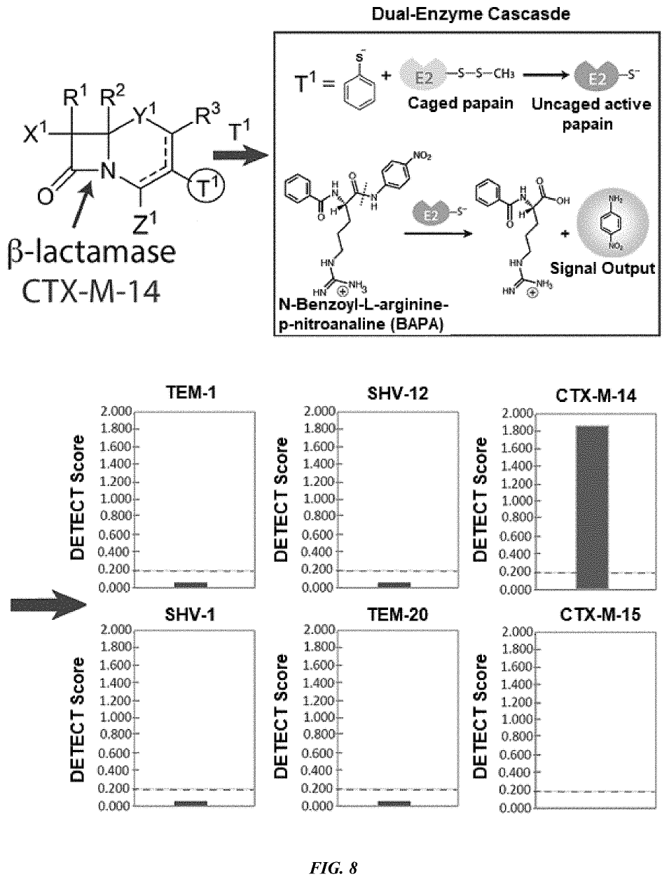

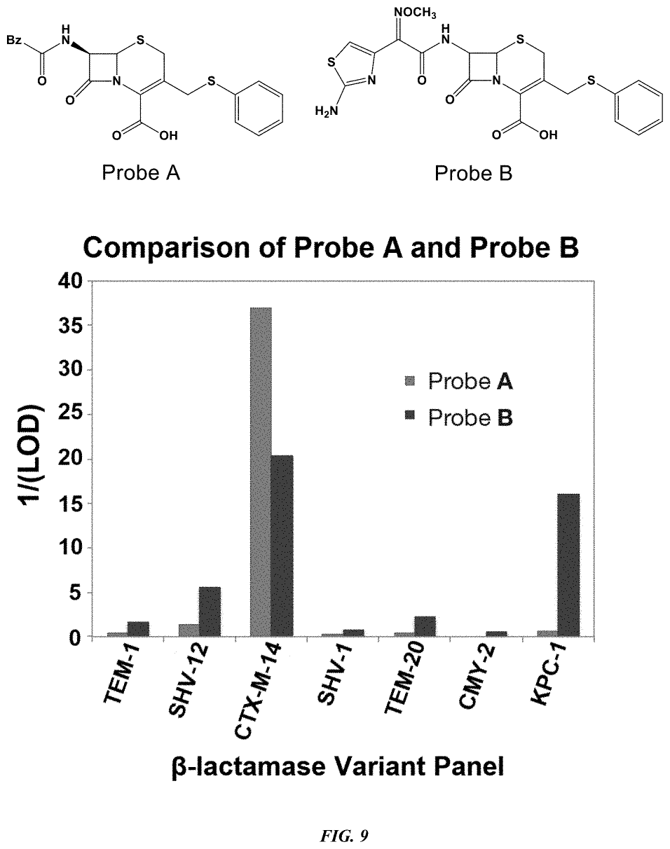

Overall, 472 urine samples were tested with DETECT, with 118 (25%) classified as representing a true UTI based on standard microbiological criteria (≥10⁴ CFU/mL cutoff applied). The urine samples tested were found to be diverse in both appearance and pH. Urine color ranged from a standard pale yellow to red; urine clarity ranged from clear to highly turbid (see FIG. 7A). Urine pH ranged from pH 5 to 9 (see FIG. 7B). Of the 118 microbiologically-defined UTIs, 96 (81%) were caused by GNB, 20 (17%) were caused by GPB, and two (2%) were caused by yeast (see FIG. 4A). Based on clinically significant CFU/mL counts, there were 109 GNB isolates from the 96 GNB UTI samples; nine urine samples grew 2 GNB species, while two samples grew 3 GNB species. The Enterobacteriaceae were the most common cause of UTI, with E. coli (73 isolates), K. pneumoniae (17), and P. mirabilis (9) being the most commonly isolated species (see FIG. 4B). Of the 118 UTIs, 13 (11%) were caused by ESBL-producing GNB, 11 (85%) of which produced a CTX-M type ESBL (see FIGS. 4C and 4D). There were nine ESBL-producing E. coli (8 CTX-M and 1 TEM ESBL), three ESBL-producing K. pneumoniae (2 CTX-M and 1 SHV ESBL), and one ESBL-producing P. mirabilis (CTX-M) (see FIG. 4D). Microbiological features, DETECT Score, and ESBL variants identified in ESBL-positive urine samples are described in see TABLE 5. The following ESBL genes were identified: nine (69%) CTX-M-15, one (8%) CTX-M-14, one (8%) CTX-M-27, one (8%) TEM-10, and one (8%) SHV-9/12 from the 13 ESBL-producing isolates.

TABLE 5
ESBL-positive urine samples tested with DETECT.

| Urine | DETECT | | | | β-lactamase |
|---|---|---|---|---|---|
| No. | score | Int.^{a} | ~CFU/mL^{b} | Organism ID | genes^{c} |
| HH-025 | 0.2600 | TP | 10^{4to5} | E. coli | CTX-M-15, |
| | | | | | TEM-1 |
| HH-055 | 1.6023 | TP | >10⁵, pure | E. coli | CTX-M-15, |
| | | | | | OXA-1 |
| HH-098 | 1.0155 | TP | >10⁵, | P. | presumed |
| | | | multiple | aeruginosa | cAmpC |
| | | | G− | E. coli | CTX-M-27 |
| | | | | P. mirabilis | ND |
| HH-099 | 1.8809 | TP | >10⁵ | K. | CTX-M-15, |
| | | | | pneumoniae | SHV-28 |
| HH-236 | X | Error | >10⁵, | K. | SHV-148 |
| | | | multiple | pneumoniae | |
| | | | G− | E. coli | TEM-10 |
| | | | | | (ESBL) |
| HH-244 | 1.9750 | TP | >10⁵, pure | E. coli | CTX-M-15, |
| | | | | | TEM-1, |
| | | | | | OXA-1 |
| HH-261 | 0.0400 | FN | 10^{4to5}, pure | K. | CTX-M-15, |
| | | | | pneumoniae | SHV-28, |
| | | | | | OXA-1 |
| HH-281 | 2.0950 | TP | >10⁵ | E. coli | CTX-M-15, |
| | | | | | OXA-1 |
| HH-293 | 0.0410 | TN | 10⁴ | K. | SHV-9/12 |
| | | | | pneumoniae | (ESBL), |
| | | | | | TEM-1 |
| HH-415 | 1.6040 | TP | >10⁵ | E. coli | CTX-M-15, |
| | | | | | OXA-1 |
| HH-434 | 0.5443 | TP | >10⁵, | K. | SHV-60 |
| | | | multiple | pneumoniae | |
| | | | G− | P. mirabilis | CTX-M-14, |
| | | | | | TEM-1 |
| HH-465 | 1.4840 | TP | >10⁵, pure | E. coli | CTX-M-15, |
| | | | | | OXA-1 |

^{a}Int., interpretation of DETECT result (threshold = 0.2588); TP, true positive; Error, DETECT Score could not be generated due to an oversaturation of signal at 30 min; FN, false-negative; TN, true negative.
^{b}“Pure” indicates the urine sample yielded a pure culture of the indicated organism. When “pure” is not indicated, the sample also contained insignificant CFU of skin/urogenital flora. G−, Gram-negative bacteria.
^{c}Presumed cAmpC indicates the species is known to contain a cAmpC. Due to their intrinsic nature, these enzymes were not tested for by PCR but were assumed to be present. ND, none detected.

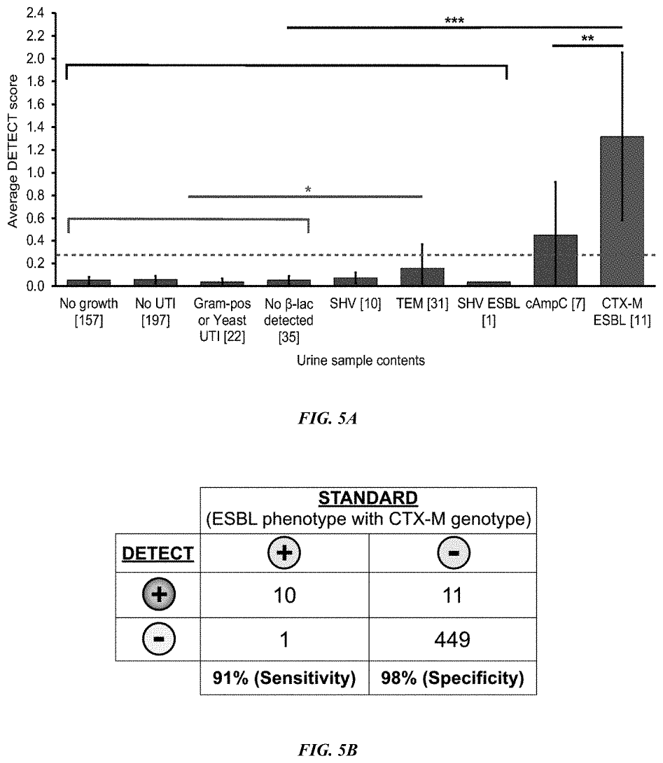

Urine samples were grouped by microbiologic contents, to evaluate DETECT Scores generated by these different types of samples (see FIG. 5A). These groups included: urine samples that did not grow bacteria (no growth); urine samples that grew bacteria that were not indicative of UTI (no UTI); urine samples from UTIs caused by GPB or yeast (Gram-pos or Yeast UTI); and urine samples from UTIs caused by GNB that contained no β-lactamase detected (No β-lactamase detected), GNB with SHV (SHV), GNB with TEM (TEM), GNB with an SHV ESBL (SHV ESBL), GNB with a chromosomal AmpC (cAmpC), or GNB with a CTX-M (CTX-M). The average DETECT Score generated by UTI samples containing CTX-M-producing GNB was 1.3, which was three times greater than the average DETECT Score generated by UTI samples containing cAmpC-producing GNB (0.44, P<0.01), and 8 to 36 times greater than the average DETECT Score generated by all other types of urine samples (0.04-0.16, P<0.001 for all). A DETECT Score could not be calculated for one urine sample—at 30 min this sample generated a signal that exceeded the spectrophotometer's detection range. Full urine sample data is provided in see TABLE 6.

TABLE 6
Clinical urine samples tested with DETECT

| | | | DETECT | | | ESBL |
|---|---|---|---|---|---|---|
| | Urine | Urine | Score | | | confirmatory |
| Urine | Appearance | CFU/mL | 30 min | Organism | β-lactamase | testing |
| No.^{a} | (clarity, color) | estimate | Urine | ID | gene list^{c} | result^{d} |
| HH-001 | Clear, pale | >10\hat{ }5, | 0.3177 | E. coli | TEM-1 | X |
| | yellow | pure | | | | |
| HH-002 | Clear, pale | NG | 0.0685 | | | |
| | yellow | | | | | |
| HH-003 | Clear, pale | >10\hat{ }5, | 0.4551 | E. coli | TEM-1 | X |
| | yellow | pure | | | | |
| HH-004 | Turbid, pale | >10\hat{ }5 | 0.0993 | E. coli | ND | X |
| | yellow | | | | | |
| HH-005 | Slightly | >10\hat{ }5 | 0.0575 | | | |
| | turbid, pink | S/GEN | | | | |
| HH-006 | Clear, pale | NG | 0.0539 | | | |
| | yellow | | | | | |
| HH-007 | Slightly | 10\hat{ }4 | 0.0851 | | | |
| | turbid, pale | S/GEN | | | | |
| | yellow | | | | | |
| HH-008 | Clear, pale | NG | 0.1099 | | | |
| | yellow | | | | | |
| HH-009 | Turbid, pale | NG | 0.0503 | | | |
| | yellow | | | | | |
| HH-010 | Turbid, pale | NG | 0.0730 | | | |
| | yellow | | | | | |
| HH-011 | Slightly | >10\hat{ }5 | 0.0115 | E. coli | TEM-1 | X |
| | turbid, pale | | | | | |
| | yellow | | | | | |
| HH-012 | Slightly | >10\hat{ }5 | 0.1212 | E. coli | SHV-1 | X |
| | turbid, pale | | | | | |
| | yellow | | | | | |
| HH-013 | Clear, pale | NG | 0.0665 | | | |
| | yellow | | | | | |
| HH-014 | Slightly | >10\hat{ }5 | 0.0916 | | | |
| | turbid, pink | S/GEN | | | | |
| HH-015 | Turbid, red | 10\hat{ }5 | 0.0872 | | | |
| | | S/GEN | | | | |
| HH-016 | Clear, pale | 10\hat{ }3 | 0.0783 | | | |
| | yellow | S/GEN | | | | |
| HH-017 | Clear, pale | NG | 0.0512 | | | |
| | yellow | | | | | |
| HH-018 | Clear, pale | >10\hat{ }5 | 0.0601 | | | |
| | yellow | S/GEN | | | | |
| HH-019 | Clear, pale | 10\hat{ }3 | 0.0604 | | | |
| | yellow | S/GEN | | | | |
| HH-020 | Turbid, pink | NG | 0.1273 | | | |
| HH-021 | Clear, pale | NG | 0.0307 | | | |
| | yellow | | | | | |
| HH-022 | Clear, pale | NG | 0.0000 | | | |
| | yellow | | | | | |
| HH-023 | Slightly | >10\hat{ }5 | 0.0291 | E. coli | ND | X |
| | turbid, pale | | | | | |
| | yellow | | | | | |
| HH-024 | Clear, | 10\hat{ }3 | 0.0192 | | | |
| | yellow/brown | S/GEN | | | | |
| HH-025 | Clear, bright | 10\hat{ }4-5 | 0.2600 | E. coli | TEM-1, | Positive |
| | orange | | | | CTX-M-15 | |
| HH-027 | Clear, pale | NG | 0.0205 | | | |
| | yellow | | | | | |
| HH-028 | Clear, | 10\hat{ }3 | 0.0384 | | | |
| | yellow/brown | S/GEN | | | | |
| HH-029 | Clear, bright | NG | 0.0104 | | | |
| | yellow | | | | | |
| HH-030 | Clear, pale | 10\hat{ }4-5 | 0.0155 | | | |
| | yellow | S/GEN | | | | |
| HH-031 | Clear, bright | 10\hat{ }3 | 0.0223 | | | |
| | yellow | S/GEN | | | | |
| HH-032 | Turbid, | NG | 0.0768 | | | |
| | bright orange | | | | | |
| HH-033 | Clear, pale | 10\hat{ }3 | 0.0317 | | | |
| | yellow | S/GEN | | | | |
| HH-034 | Turbid, | >10\hat{ }5, | 0.0000 | E. faecalis | | |
| | bright orange | pure | | | | |
| HH-035 | Clear, bright | 10\hat{ }4 | 0.0125 | | | |
| | orange | S/GEN | | | | |
| HH-036 | Turbid, pale | NG | 0.0414 | | | |
| | yellow | | | | | |
| HH-037-1 | Clear, pale | 10\hat{ }4 | 0.0320 | E. coli | TEM-1 | X |
| | yellow | multiple | | | | |
| | | G− | | | | |
| HH-037-2 | | | | E. coli | ND | X |
| HH-038 | Clear, pale | 10\hat{ }3 | 0.0594 | | | |
| | yellow | S/GEN | | | | |
| HH-039 | Clear, pale | NG | 0.0573 | | | |
| | yellow | | | | | |
| HH-040 | Clear, pale | NG | 0.0383 | | | |
| | yellow | | | | | |
| HH-041 | Slightly | 10\hat{ }3 | 0.0493 | | | |
| | turbid, pale | S/GEN | | | | |
| | yellow | | | | | |
| HH-042 | Slightly | >10\hat{ }5 | 0.0045 | E. coli | ND | X |
| | turbid, pale | | | | | |
| | yellow | | | | | |
| HH-043 | Turbid, pale | 10\hat{ }4 | 0.0916 | | | |
| | yellow | S/GEN | | | | |
| HH-044 | Clear, pale | 10\hat{ }4 | 0.0635 | S. epidermidis | | |
| | yellow | | | | | |
| HH-045 | Clear, pale | NG | 0.0491 | | | |
| | yellow | | | | | |
| HH-046 | Clear, bright | NG | 0.0468 | | | |
| | orange | | | | | |
| HH-047 | Clear, pale | 10\hat{ }4 | 0.0271 | | | |
| | yellow | S/GEN | | | | |
| HH-048 | Clear, pale | 10\hat{ }3 | 0.0346 | | | |
| | yellow | S/GEN | | | | |
| HH-049 | Clear, pink | 10\hat{ }4 | 0.0174 | | | |
| | | S/GEN | | | | |
| HH-050 | Clear, pale | NG | 0.0161 | | | |
| | yellow | | | | | |
| HH-051 | Clear, pale | 10\hat{ }4 | 0.0400 | | | |
| | yellow | S/GEN | | | | |
| HH-052 | Clear, pale | NG | 0.0476 | | | |
| | yellow | | | | | |
| HH-053 | Clear, pale | NG | 0.0353 | | | |
| | yellow | | | | | |
| HH-054 | Clear, pale | 10\hat{ }4 | 0.0409 | | | |
| | yellow | S/GEN | | | | |
| HH-055 | Clear, pale | >10\hat{ }5, | 1.6023 | E. coli | OXA-1, | Positive |
| | yellow | pure | | | CTX-M-15 | |
| HH-056 | Clear, pale | 10\hat{ }3 | 0.0997 | | | |
| | yellow | S/GEN | | | | |
| HH-057 | Clear, pale | 10\hat{ }4 | 0.0477 | K. oxytoca | ND | X |
| | yellow | | | | | |
| HH-058 | Clear, pale | NG | 0.0242 | | | |
| | yellow | | | | | |
| HH-059 | Clear, pale | NG | 0.0442 | | | |
| | yellow | | | | | |
| HH-060 | Clear, pale | 10\hat{ }3 | 0.0494 | | | |
| | yellow | S/GEN | | | | |
| HH-061 | Clear, pale | >10\hat{ }5, | 0.0396 | E. coli | TEM-1 | X |
| | yellow | pure | | | | |
| HH-062 | Clear, pale | NG | 0.0641 | | | |
| | yellow | | | | | |
| HH-063 | Clear, pale | >10\hat{ }5, | 0.0913 | E. coli | ND | X |
| | yellow | pure | | | | |
| HH-064 | Clear, pale | NG | 0.1017 | | | |
| | yellow | | | | | |
| HH-065 | Clear, pale | 10\hat{ }3 | 0.1164 | | | |
| | yellow | S/GEN | | | | |
| HH-066 | Clear, pale | 10\hat{ }4 | 0.0112 | | | |
| | yellow | S/GEN | | | | |
| HH-067 | Clear, pale | NG | 0.0711 | | | |
| | yellow | | | | | |
| HH-068 | Turbid, pale | >10\hat{ }5 | 0.5805 | E. coli | TEM-1 | X |
| | yellow | | | | | |
| HH-069 | Clear, pale | 10\hat{ }5 | 0.1096 | | | |
| | yellow | S/GEN | | | | |
| HH-070 | Clear, pale | NG | 0.0875 | | | |
| | yellow | | | | | |
| HH-071 | Clear, pale | 10\hat{ }4 | 0.0896 | | | |
| | yellow | S/GEN | | | | |
| HH-072 | Slightly | 10\hat{ }4 | 0.0827 | E. coli | ND | X |
| | turbid, pale | | | | | |
| | yellow | | | | | |
| HH-073 | Clear, pale | NG | 0.0594 | | | |
| | yellow | | | | | |
| HH-074 | Clear, pale | 10\hat{ }3 | 0.0363 | | | |
| | yellow | S/GEN | | | | |
| HH-075 | Clear, pale | NG | 0.0759 | | | |
| | yellow | | | | | |
| HH-076 | Turbid, pale | >10\hat{ }5 | 0.0339 | | | |
| | yellow | S/GEN | | | | |
| HH-077 | Clear, pale | NG | 0.0823 | | | |
| | yellow | | | | | |
| HH-078 | Clear, pale | >10\hat{ }5, | 0.0348 | E. coli | ND | X |
| | yellow | pure | | | | |
| HH-079 | Clear, pale | NG | 0.1005 | | | |
| | yellow | | | | | |
| HH-080 | Clear, pale | >10\hat{ }5 | 0.1835 | | | |
| | yellow | S/GEN | | | | |
| HH-081 | Clear, bright | >10\hat{ }5 | 0.1147 | E. coli | TEM-1 | X |
| | yellow | | | | | |
| HH-082 | Clear, bright | NG | 0.0352 | | | |
| | yellow | | | | | |
| HH-083 | Clear, pale | 10\hat{ }3 | 0.1064 | | | |
| | yellow | S/GEN | | | | |
| HH-084 | Turbid, pale | NG | 0.1047 | | | |
| | yellow | | | | | |
| HH-085 | Clear, pale | NG | 0.0451 | | | |
| | yellow | | | | | |
| HH-086 | Clear, pale | 10\hat{ }3 | 0.0651 | | | |
| | yellow | S/GEN | | | | |
| HH-087 | Clear, pale | 10\hat{ }5 | 0.0857 | | | |
| | yellow | S/GEN | | | | |
| HH-088 | Clear, pale | 10\hat{ }3 | 0.0620 | | | |
| | yellow | S/GEN | | | | |
| HH-089 | Clear, bright | NG | 0.0847 | | | |
| | yellow | | | | | |
| HH-090 | Clear, pale | NG | 0.1347 | | | |
| | yellow | | | | | |
| HH-091 | Clear, pale | 10\hat{ }5 | 0.1051 | | | |
| | yellow | S/GEN | | | | |
| HH-092 | Clear, pale | 10\hat{ }5 | 0.0968 | | | |
| | yellow | S/GEN | | | | |
| HH-093 | Clear, pale | 10\hat{ }3 | 0.0828 | | | |
| | yellow | S/GEN | | | | |
| HH-094 | Clear, pale | 10\hat{ }4-5 | 0.0561 | S. aureus | | |
| | yellow | | | | | |
| HH-095 | Clear, pale | 10\hat{ }3 | 0.0944 | | | |
| | yellow | S/GEN | | | | |
| HH-096 | Clear, pale | NG | 0.1204 | | | |
| | yellow | | | | | |
| HH-097 | Clear, pale | NG | 0.0894 | | | |
| | yellow | | | | | |
| HH-098-1 | Clear, pale | >10\hat{ }5 | 1.0155 | P. aeruginosa | presumed | Negative |
| | yellow | multiple | | | cAmpC: ND | |
| | | G− | | | for others | |
| HH-098-2 | | | | E. coli | CTX-M-27 | Positive |
| HH-098-3 | | | | P. mirabilis | ND | X |
| HH-099 | Clear, pale | >10\hat{ }5 | 1.8809 | K. | SHV-28, | Positive |
| | yellow | | | pneumoniae | CTX-M-15 | |
| HH-100 | Turbid, pale | NG | 0.0605 | | | |
| | yellow | | | | | |
| HH-101 | Clear, pale | NG | 0.0912 | | | |
| | yellow | | | | | |
| HH-102 | Clear, bright | NG | 0.0210 | | | |
| | yellow | | | | | |
| HH-103 | Clear, pale | >10\hat{ }5, | 0.1196 | E. coli | ND | X |
| | yellow | pure | | | | |
| HH-104 | Clear, pale | 10\hat{ }3 | 0.0776 | | | |
| | yellow | S/GEN | | | | |
| HH-105 | Clear, pale | >10\hat{ }5 | 0.0396 | Group B | | |
| | yellow | | | Streptococcus | | |
| HH-106 | Clear, pale | NG | 0.0980 | | | |
| | yellow | | | | | |
| HH-107 | Clear, pale | NG | 0.1274 | | | |
| | yellow | | | | | |
| HH-108 | Clear, pale | >10\hat{ }5 | 0.0582 | | | |
| | yellow | S/GEN | | | | |
| HH-109 | Clear, bright | NG | 0.0829 | | | |
| | yellow | | | | | |
| HH-110 | Clear, bright | NG | 0.0150 | | | |
| | yellow | | | | | |
| HH-111 | Clear, pale | NG | 0.0926 | | | |
| | yellow | | | | | |
| HH-112 | Turbid, pale | >10\hat{ }5 | 0.1211 | | | |
| | yellow | S/GEN | | | | |
| HH-113 | Clear, pale | 10\hat{ }3 | 0.1215 | | | |
| | yellow | S/GEN | | | | |
| HH-114 | Clear, pale | >10\hat{ }5 | 0.1339 | Group B | | |
| | yellow | | | Streptococcus | | |
| HH-115 | Clear, bright | NG | 0.0443 | | | |
| | yellow | | | | | |
| HH-116 | Turbid, pale | 10\hat{ }4 | 0.1120 | E. coli | TEM-1 | X |
| | yellow | | | | | |
| HH-117 | Clear, pale | >10\hat{ }5 | 0.0579 | | | |
| | yellow | S/GEN | | | | |
| HH-118 | Clear, pale | NG | 0.0097 | | | |
| | yellow | | | | | |
| HH-119 | Clear, pale | 10\hat{ }4 | 0.0206 | | | |
| | yellow | S/GEN | | | | |
| HH-120 | Clear, pale | 10\hat{ }4-5 | 0.0387 | Coagulase- | | |
| | yellow | | | negative | | |
| | | | | Staphylococcus | | |
| HH-121 | Clear, pale | 10\hat{ }3 | 0.0109 | | | |
| | yellow | S/GEN | | | | |
| HH-122 | Clear, pale | 10\hat{ }4 | 0.0929 | | | |
| | yellow | S/GEN | | | | |
| HH-123 | Clear, pale | NG | 0.0330 | | | |
| | yellow | | | | | |
| HH-124 | Clear, pale | NG | 0.0919 | | | |
| | yellow | | | | | |
| HH-125 | Clear, pale | 10\hat{ }4 | 0.0363 | | | |
| | yellow | S/GEN | | | | |
| HH-126 | Turbid, red | NG | 0.0427 | | | |
| HH-127 | Clear, pale | >10\hat{ }5 | 0.0884 | E. coli | ND | X |
| | yellow | | | | | |
| HH-128-1 | Clear, pale | >10\hat{ }5 | 0.2914 | E. coli | TEM-1 | X |
| | yellow | multiple | | | | |
| | | G− | | | | |
| HH-128-2 | | | | K. | SHV-11 | X |
| | | | | pneumoniae | | |
| HH-128-3 | | | | P. mirabilis | ND | X |
| HH-129 | Clear, pale | 10\hat{ }3 | 0.0276 | | | |
| | yellow | S/GEN | | | | |
| HH-130 | Clear, pale | NG | 0.0781 | | | |
| | yellow | | | | | |
| HH-131 | Clear, pale | >10\hat{ }5, | 0.2724 | E. coli | TEM-1 | Negative |
| | yellow | pure | | | | |
| HH-132 | Clear, pale | 10\hat{ }4 | 0.0604 | | | |
| | yellow | S/GEN | | | | |
| HH-133 | Clear, pale | 10\hat{ }3 | 0.0375 | | | |
| | yellow | S/GEN | | | | |
| HH-134 | Clear, pale | >10\hat{ }5 | 0.0503 | | | |
| | yellow | S/GEN | | | | |
| HH-135 | Clear, pale | 10\hat{ }3 | 0.0238 | | | |
| | yellow | S/GEN | | | | |
| HH-136 | Clear, pale | NG | 0.0388 | | | |
| | yellow | | | | | |
| HH-137 | Clear, pale | >10\hat{ }5 | 0.0542 | E. coli | TEM-1 | X |
| | yellow | | | | | |
| HH-138 | Clear, pale | NG | 0.0496 | | | |
| | yellow | | | | | |
| HH-139 | Clear, pale | NG | 0.0454 | | | |
| | yellow | | | | | |
| HH-140 | Clear, pale | NG | 0.0536 | | | |
| | yellow | | | | | |
| HH-141 | Clear, pale | NG | 0.0316 | | | |
| | yellow | | | | | |
| HH-142 | Clear, pale | >10\hat{ }5 | 0.0409 | | | |
| | yellow | S/GEN | | | | |
| HH-144 | Clear, pale | >10\hat{ }5 | 0.0383 | E. coli | ND | X |
| | yellow | | | | | |
| HH-145 | Clear, pale | 10\hat{ }4-5, | 0.0308 | Lactobacillus | | |
| | yellow | pure | | sp. | | |
| HH-146 | Clear, pale | 10\hat{ }5, | 0.0438 | E. coli | TEM-1 | X |
| | yellow | pure | | | | |
| HH-147 | Clear, pale | >10\hat{ }5 | 0.0785 | | | |
| | yellow | S/GEN | | | | |
| HH-148 | Clear, pale | 10\hat{ }4 | 0.0716 | | | |
| | yellow | S/GEN | | | | |
| HH-149 | Clear, pale | NG | 0.0772 | | | |
| | yellow | | | | | |
| HH-150 | Clear, pale | 10\hat{ }4 | 0.0281 | | | |
| | yellow | S/GEN | | | | |
| HH-151 | Clear, pale | 10\hat{ }4 | 0.0337 | | | |
| | yellow | S/GEN | | | | |
| HH-152 | Turbid, | 10\hat{ }5 | 0.0374 | | | |
| | bright yellow | S/GEN | | | | |
| HH-153 | Clear, pale | NG | 0.0285 | | | |
| | yellow | | | | | |
| HH-154 | Clear, pale | 10\hat{ }5 | 0.0317 | | | |
| | yellow | S/GEN | | | | |
| HH-155 | Turbid, | 10\hat{ }5 | 0.0373 | | | |
| | bright yellow | S/GEN | | | | |
| HH-156 | Clear, bright | NG | 0.0016 | | | |
| | yellow | | | | | |
| HH-157 | Clear, pale | 10\hat{ }3 | 0.0260 | | | |
| | yellow | S/GEN | | | | |
| HH-158 | Clear, pale | 10\hat{ }5 | 0.0426 | | | |
| | yellow | S/GEN | | | | |
| HH-159 | Turbid, pale | NG | 0.1256 | | | |
| | yellow | | | | | |
| HH-160 | Clear, pale | 10\hat{ }5 | 0.1452 | | | |
| | yellow | S/GEN | | | | |
| HH-161 | Clear, pale | 10\hat{ }5 | 0.0321 | | | |
| | yellow | S/GEN | | | | |
| HH-162 | Clear, pale | NG | 0.0357 | | | |
| | yellow | | | | | |
| HH-163 | Clear, pale | 10\hat{ }4-5 | 0.0943 | E. aerogenes | presumed | X |
| | yellow | | | | cAmpC: ND | |
| | | | | | for others | |
| HH-164 | Clear, pale | 10\hat{ }5 | 0.0418 | | | |
| | yellow | S/GEN | | | | |
| HH-165 | Turbid, | 10\hat{ }5 | 0.2608 | | | |
| | bright orange | S/GEN | | | | |
| HH-166 | Clear, pale | NG | 0.0332 | | | |
| | yellow | | | | | |
| HH-167 | Clear, pale | 10\hat{ }4 | 0.0411 | | | |
| | yellow | S/GEN | | | | |
| HH-168 | Clear, pale | NG | 0.0264 | | | |
| | yellow | | | | | |
| HH-169 | Clear, pale | NG | 0.0337 | | | |
| | yellow | | | | | |
| HH-170 | Clear, pale | 10\hat{ }4 | 0.0392 | | | |
| | yellow | S/GEN | | | | |
| HH-171 | Clear, pale | NG | 0.0321 | | | |
| | yellow | | | | | |
| HH-172 | Turbid, pale | NG | 0.0452 | | | |
| | yellow | | | | | |
| HH-173 | Clear, pale | >10\hat{ }5 | 0.0351 | E. coli | TEM-1 | X |
| | yellow | | | | | |
| HH-174 | Clear, pale | 10\hat{ }4 | 0.0141 | E. faecalis | | |
| | yellow | | | | | |
| HH-175 | Clear, pale | NG | 0.0146 | | | |
| | yellow | | | | | |
| HH-176 | Clear, pale | 10\hat{ }5 | 0.0379 | | | |
| | yellow | S/GEN | | | | |
| HH-177 | Slightly | >10\hat{ }5 | 0.1264 | E. coli | ND | X |
| | turbid, red | | | | | |
| HH-178 | Clear, pale | NG | 0.0551 | | | |
| | yellow | | | | | |
| HH-179 | Clear, bright | >10\hat{ }5, | 0.0154 | E. coli | TEM-1 | X |
| | yellow | pure | | | | |
| HH-180 | Clear, pale | >10\hat{ }5 | 0.1267 | E. coli | ND | X |
| | yellow | | | | | |
| HH-181 | Clear, pale | 10\hat{ }4, | 0.0327 | E. coli | ND | X |
| | yellow | pure | | | | |
| HH-182 | Clear, pale | 10\hat{ }4 | 0.0199 | | | |
| | yellow | S/GEN | | | | |
| HH-183 | Clear, pale | 10\hat{ }5 | 0.0357 | | | |
| | yellow | S/GEN | | | | |
| HH-184 | Clear, pale | 10\hat{ }4 | 0.0305 | | | |
| | yellow | S/GEN | | | | |
| HH-185 | Clear, bright | NG | 0.0063 | | | |
| | yellow | | | | | |
| HH-186 | Clear, pale | 10\hat{ }4 | 0.0484 | | | |
| | yellow | S/GEN | | | | |
| HH-187 | Clear, bright | 10\hat{ }3 | 0.0324 | | | |
| | yellow | S/GEN | | | | |
| HH-188 | Clear, pale | NG | 0.0246 | | | |
| | yellow | | | | | |
| HH-189 | Clear, pale | NG | 0.0514 | | | |
| | yellow | | | | | |
| HH-190 | Clear, pink | 10\hat{ }5 | 0.0804 | | | |
| | | S/GEN | | | | |
| HH-191 | Clear, pale | >10\hat{ }5, | 0.2575 | E. aerogenes | presumed | X |
| | yellow | pure | | | cAmpC: ND | |
| | | | | | for others | |
| HH-192 | Clear, pale | >10\hat{ }5, | 0.0512 | E. coli | TEM-1 | X |
| | yellow | pure | | | | |
| HH-193 | Clear, pale | 10\hat{ }4-5 | 0.0127 | E. coli | TEM-1 | X |
| | yellow | | | | | |
| HH-194 | Clear, pale | 10\hat{ }3 | 0.0473 | | | |
| | yellow | S/GEN | | | | |
| HH-195 | Clear, pale | 10\hat{ }4 | 0.0523 | | | |
| | yellow | S/GEN | | | | |
| HH-196 | Clear, pale | NG | 0.0344 | | | |
| | yellow | | | | | |
| HH-197 | Clear, pale | NG | 0.0856 | | | |
| | yellow | | | | | |
| HH-198 | Turbid, red | 10\hat{ }4 | 0.0883 | | | |
| | | S/GEN | | | | |
| HH-199 | Clear, pale | 10\hat{ }4-5 | 0.0729 | E. coli | TEM-1 | X |
| | yellow | | | | | |
| HH-200 | Clear, pale | NG | 0.0515 | | | |
| | yellow | | | | | |
| HH-201 | Slightly | NG | 0.0433 | | | |
| | turbid, pale | | | | | |
| | yellow | | | | | |
| HH-202 | Clear, pale | NG | 0.0185 | | | |
| | yellow | | | | | |
| HH-203-1 | Clear, pale | >10\hat{ }5 | 0.0938 | K. | SHV-28/83 | X |
| | yellow | multiple | | pneumoniae | | |
| | | G− | | | | |
| HH-203-2 | | | | P. mirabilis | ND | X |
| HH-204 | Clear, pale | 10\hat{ }4-5 | 0.0150 | | | |
| | yellow | S/GEN | | | | |
| HH-205 | Clear, pale | 10\hat{ }4 | 0.0373 | | | |
| | yellow | S/GEN | | | | |
| HH-206 | Clear, pale | >10\hat{ }5 | 0.0322 | S. epidermidis | | |
| | yellow | | | | | |
| HH-207 | Clear, pale | NG | 0.0181 | | | |
| | yellow | | | | | |
| HH-208 | Clear, bright | NG | 0.0364 | | | |
| | yellow | | | | | |
| HH-209 | Clear, pale | NG | 0.0365 | | | |
| | yellow | | | | | |
| HH-210 | Clear, pale | 10\hat{ }4 | 0.0291 | | | |
| | yellow | S/GEN | | | | |
| HH-211 | Clear, pale | 10\hat{ }4-5 | 0.0554 | E. coli | ND | X |
| | yellow | | | | | |
| HH-212 | Clear, pale | 10\hat{ }4-5 | 0.0511 | | | |
| | yellow | | | | | |
| HH-213 | Clear, pale | NG | 0.0426 | | | |
| | yellow | | | | | |
| HH-214 | Clear, pale | NG | 0.0511 | | | |
| | yellow | | | | | |
| HH-215 | Slightly | NG | 0.0713 | | | |
| | turbid, bright | | | | | |
| | yellow | | | | | |
| HH-216 | Clear, pale | NG | 0.0583 | | | |
| | yellow | | | | | |
| HH-217 | Clear, pale | 10\hat{ }4-5 | 0.0323 | | | |
| | yellow | S/GEN | | | | |
| HH-218 | Clear, bright | 10\hat{ }3 | 0.0444 | | | |
| | yellow | | | | | |
| HH-219 | Clear, pale | NG | 0.0227 | | | |
| | yellow | | | | | |
| HH-220 | Clear, pale | NG | 0.0365 | | | |
| | yellow | | | | | |
| HH-221 | Clear, pale | 10\hat{ }4 | 0.0379 | | | |
| | yellow | S/GEN | | | | |
| HH-222 | Clear, pale | NG | 0.0319 | | | |
| | yellow | | | | | |
| HH-223 | Clear, pale | >10\hat{ }5 | 0.0463 | K. | LEN | X |
| | yellow | | | pneumoniae | (detected | |
| | | | | | by SHV | |
| | | | | | primers) | |
| HH-224 | Clear, pale | 10\hat{ }4-5 | 0.1240 | | | |
| | yellow | S/GEN | | | | |
| HH-225 | Clear, pale | 10\hat{ }4-5 | 0.1203 | | | |
| | yellow | S/GEN | | | | |
| HH-226 | Clear, pale | 10\hat{ }5 | 0.0308 | | | |
| | yellow | S/GEN | | | | |
| HH-227 | Clear, pale | NG | 0.0242 | | | |
| | yellow | | | | | |
| HH-228 | Clear, pale | NG | 0.0558 | | | |
| | yellow | | | | | |
| HH-229 | Clear, pale | 10\hat{ }4 | 0.0978 | | | |
| | yellow | S/GEN | | | | |
| HH-230 | Clear, pale | NG | 0.0325 | | | |
| | yellow | | | | | |
| HH-231 | Clear, pale | 10\hat{ }4 | 0.0368 | S. bovis | | |
| | yellow | | | | | |
| HH-232 | Turbid, | 10\hat{ }4 | 0.0681 | | | |
| | bright yellow | S/GEN | | | | |
| HH-233 | Clear, pale | 10\hat{ }4-5 | 0.0968 | | | |
| | yellow | S/GEN | | | | |
| HH-234 | Clear, pale | NG | 0.0422 | | | |
| | yellow | | | | | |
| HH-235 | Slightly | 10\hat{ }4 | 0.0584 | | | |
| | turbid, pale | S/GEN | | | | |
| | yellow | | | | | |
| HH-236-1 | Red, clear | 10\hat{ }5 | X (could | K. | SHV-148 | X |
| | | multiple | not obtain | pneumoniae | | |
| | | G− | score) | | | |
| HH-236-2 | | | | E. coli | TEM-10 | Positive |
| HH-237 | Clear, pale | >10\hat{ }5 | 0.0150 | E. coli | ND | X |
| | yellow | | | | | |
| HH-238 | Clear, pale | 10\hat{ }4 | 0.0358 | | | |
| | yellow | S/GEN | | | | |
| HH-239 | Clear, pale | >10\hat{ }5 | 0.0006 | Yeast | | |
| | yellow | | | | | |
| HH-240 | Clear, pale | 10\hat{ }3 | 0.0306 | | | |
| | yellow | S/GEN | | | | |
| HH-241 | Clear, pale | 10\hat{ }3 | 0.0417 | | | |
| | yellow | S/GEN | | | | |
| HH-242 | Turbid, pale | 10\hat{ }3 | 0.0552 | | | |
| | yellow | S/GEN | | | | |
| HH-243 | Clear, pale | >10\hat{ }5 | 0.0546 | | | |
| | yellow | S/GEN | | | | |
| HH-244 | Clear, pale | >10\hat{ }5, | 1.9750 | E. coli | TEM-1, | Positive |
| | yellow | pure | | | OXA-1, | |
| | | | | | CTX-M-15 | |
| HH-245 | Clear, pale | 10\hat{ }3 | 0.0836 | | | |
| | yellow | S/GEN | | | | |
| HH-246 | Clear, pale | NG | 0.0218 | | | |
| | yellow | | | | | |
| HH-247 | Clear, pale | NG | 0.0691 | | | |
| | yellow | | | | | |
| HH-248 | Clear, pale | >10\hat{ }5, | 0.1333 | E. coli | TEM-1 | X |
| | yellow | pure | | | | |
| HH-249 | Clear, pale | 10\hat{ }3 | 0.0368 | | | |
| | yellow | S/GEN | | | | |
| HH-250 | Clear, pale | >10\hat{ }5 | 0.0364 | E. coli | TEM-1 | X |
| | yellow | | | | | |
| HH-251 | Clear, pale | 10\hat{ }4 | 0.0501 | | | |
| | yellow | S/GEN | | | | |
| HH-252 | Clear, pale | NG | 0.0707 | | | |
| | yellow | | | | | |
| HH-253 | Clear, pale | >10\hat{ }5, | 0.0769 | E. coli | TEM-1 | X |
| | yellow | pure | | | | |
| HH-254 | Clear, pale | NG | 0.0305 | | | |
| | yellow | | | | | |
| HH-255 | Clear, pale | 10\hat{ }4 | 0.0266 | | | |
| | yellow | S/GEN | | | | |
| HH-256 | Clear, pale | 10\hat{ }4-5, | 0.0134 | E. coli | ND | X |
| | yellow | pure | | | | |
| HH-257 | Clear, pale | NG | 0.0426 | | | |
| | yellow | | | | | |
| HH-258 | Clear, pale | >10\hat{ }5 | 0.0417 | S. | | |
| | yellow | | | saprophyticus | | |
| HH-259 | Clear, pale | 10\hat{ }3 | 0.0629 | | | |
| | yellow | S/GEN | | | | |
| HH-260 | Clear, pale | 10\hat{ }4-5 | 0.0454 | K. oxytoca | ND | X |
| | yellow | | | | | |
| HH-261 | Clear, pale | 10\hat{ }4-5, | 0.0400 | K. | SHV-28, | Positive |
| | yellow | pure | | pneumoniae | OXA-1, | |
| | | | | | CTX-M-15 | |
| HH-262-1 | Clear, pale | 10\hat{ }4-5 | 0.1493 | E. coli | ND | X |
| | yellow | multiple | | | | |
| | | G− | | | | |
| HH-262-2 | | | | K. | SHV-83/187 | X |
| | | | | pneumoniae | | |
| HH-263 | Clear, pale | 10\hat{ }4-5 | 0.0797 | | | |
| | yellow | S/GEN | | | | |
| HH-264 | Clear, pale | 10\hat{ }4-5 | 0.0447 | | | |
| | yellow | S/GEN | | | | |
| HH-265 | Clear, pale | NG | 0.0418 | | | |
| | yellow | | | | | |
| HH-266 | Turbid, pale | NG | 0.1062 | | | |
| | yellow | | | | | |
| HH-267 | Clear, pale | 10\hat{ }3 | 0.0448 | | | |
| | yellow | S/GEN | | | | |
| HH-268 | Clear, pale | NG | 0.0201 | | | |
| | yellow | | | | | |
| HH-269 | Clear, pale | >10\hat{ }5, | 0.0508 | E. coli | TEM-1 | X |
| | yellow | pure | | | | |
| HH-270 | Clear, pale | NG | 0.0570 | | | |
| | yellow | | | | | |
| HH-271 | Clear, pale | NG | 0.0342 | | | |
| | yellow | | | | | |
| HH-272 | Clear, pale | 10\hat{ }3 | 0.0453 | | | |
| | yellow | S/GEN | | | | |
| HH-273 | Clear, pale | 10\hat{ }3 | 0.0555 | | | |
| | yellow | S/GEN | | | | |
| HH-274 | Clear, pale | >10\hat{ }5, | 0.0000 | K. | SHV-36 | X |
| | yellow | pure | | pneumoniae | | |
| HH-275 | Clear, pale | >10\hat{ }5 | 0.0280 | | | |
| | yellow | S/GEN | | | | |
| HH-276 | Clear, pale | 10\hat{ }4 | 0.0377 | | | |
| | yellow | S/GEN | | | | |
| HH-277 | Clear, bright | NG | 0.0827 | | | |
| | yellow | | | | | |
| HH-278 | Clear, pale | 10\hat{ }4-5 | 0.0103 | | | |
| | yellow | S/GEN | | | | |
| HH-280 | Clear, pale | NG | 0.0408 | | | |
| | yellow | | | | | |
| HH-281 | Clear, pale | >10\hat{ }5 | 2.0950 | E. coli | OXA-1, | Positive |
| | yellow | | | | CTX-M-15 | |
| HH-282 | Clear, pale | >10\hat{ }5 | 0.0523 | K. | ND | X |
| | yellow | | | pneumoniae | | |
| HH-283 | Clear, pale | 10\hat{ }4 | 0.0636 | | | |
| | yellow | S/GEN | | | | |
| HH-284 | Clear, pale | NG | 0.0343 | | | |
| | yellow | | | | | |
| HH-285 | Clear, bright | >10\hat{ }5 | 0.0099 | P. | ND | X |
| | yellow | | | agglomerans | | |
| HH-286 | Clear, pale | 10\hat{ }4 | 0.0726 | | | |
| | yellow | S/GEN | | | | |
| HH-287 | Clear, pale | NG | 0.0420 | | | |
| | yellow | | | | | |
| HH-288 | Clear, pale | 10\hat{ }4-5 | 0.0399 | | | |
| | yellow | S/GEN | | | | |
| HH-289 | Clear, pale | 10\hat{ }4 | 0.0268 | | | |
| | yellow | S/GEN | | | | |
| HH-290 | Turbid, pale | 10\hat{ }3 | 0.0831 | | | |
| | yellow | S/GEN | | | | |
| HH-291 | Clear, pale | 10\hat{ }3 | 0.0167 | | | |
| | yellow | S/GEN | | | | |
| HH-292 | Turbid, pale | NG | 0.0647 | | | |
| | yellow | | | | | |
| HH-293 | Clear, pale | 10\hat{ }4 | 0.0410 | K. | TEM-1, | Positive |
| | yellow | | | pneumoniae | SHV- | |
| | | | | | 9/12/129 | |
| | | | | | ESBL | |
| HH-294 | Slightly | 10\hat{ }4-5, | 0.0308 | E. coli | ND | X |
| | turbid, pale | pure | | | | |
| | yellow | | | | | |
| HH-295 | Clear, pale | 10\hat{ }4 | 0.0486 | | | |
| | yellow | S/GEN | | | | |
| HH-296 | Clear, pale | NG | 0.0333 | | | |
| | yellow | | | | | |
| HH-297 | Turbid, red | >10\hat{ }5 | 0.8374 | P. rettgeri | ND | X |
| | | morpho | | | | |
| | | variants | | | | |
| HH-298 | Clear, pale | >10\hat{ }5 | 0.0279 | E. coli | ND | X |
| | yellow | | | | | |
| HH-299 | Clear, pale | 10\hat{ }3, | 0.0443 | | | |
| | yellow | pure | | | | |
| HH-300 | Clear, pale | 10\hat{ }3, | 0.0714 | | | |
| | yellow | S/GEN | | | | |
| HH-301 | Clear, pale | NG | 0.0235 | | | |
| | yellow | | | | | |
| HH-302 | Clear, pale | 10\hat{ }4 | 0.0291 | | | |
| | yellow | S/GEN | | | | |
| HH-303 | Clear, pale | 10\hat{ }4 | 0.0483 | | | |
| | yellow | S/GEN | | | | |
| HH-304 | Clear, pale | NG | 0.0468 | | | |
| | yellow | | | | | |
| HH-305 | Clear, pale | >10\hat{ }5, | 0.0422 | E. coli | TEM-1 | X |
| | yellow | pure | | | | |
| HH-306 | Clear, pale | 10\hat{ }4 | 0.0416 | | | |
| | yellow | S/GEN | | | | |
| HH-307 | Clear, pale | NG | 0.0460 | | | |
| | yellow | | | | | |
| HH-308 | Clear, pale | NG | 0.0701 | | | |
| | yellow | | | | | |
| HH-309 | Clear, pale | NG | 0.0581 | | | |
| | yellow | | | | | |
| HH-310 | Clear, bright | NG | 0.0334 | | | |
| | yellow | | | | | |
| HH-311 | Turbid, pale | 10\hat{ }4 | 0.0724 | | | |
| | yellow | S/GEN | | | | |
| HH-312 | Slightly | 10\hat{ }4 | 0.0068 | | | |
| | turbid, bright | S/GEN | | | | |
| | yellow | | | | | |
| HH-313 | Clear, pale | >10\hat{ }5, | 0.0827 | E. coli | ND | X |
| | yellow | pure | | | | |
| HH-314 | Turbid, pale | >10\hat{ }5 | 0.0000 | Yeast | | |
| | yellow | | | | | |
| HH-315 | Clear, pale | 10\hat{ }4 | 0.0427 | | | |
| | yellow | S/GEN | | | | |
| HH-316 | Clear, pale | NG | 0.0181 | | | |
| | yellow | | | | | |
| HH-318 | Clear, pale | 10\hat{ }3, | 0.0243 | | | |
| | yellow | S/GEN | | | | |
| HH-319 | Turbid, pale | 10\hat{ }4-5 | 0.0000 | E. coli | ND | X |
| | yellow | | | | | |
| HH-320 | Clear, pale | >10\hat{ }5 | 0.0000 | E. coli | ND | X |
| | yellow | | | | | |
| HH-321 | Turbid, | >10\hat{ }5, | 0.0457 | K. | LEN | X |
| | bright yellow | pure | | pneumoniae | (detected by | |
| | | | | | SHV | |
| | | | | | primers) | |
| HH-322 | Turbid, pale | 10\hat{ }3, | 0.0502 | | | |
| | yellow | S/GEN | | | | |
| HH-323 | Clear, pale | 10\hat{ }4 | 0.0440 | | | |
| | yellow | S/GEN | | | | |
| HH-324 | Clear, pale | 10\hat{ }4-5, | 0.0433 | | | |
| | yellow | S/GEN | | | | |
| HH-325 | Clear, pale | 10\hat{ }5 | 0.0229 | Lactobacillus | | |
| | yellow | | | sp. | | |
| HH-326 | Slightly | >10\hat{ }5, | 0.1280 | E. coli | TEM-1 | X |
| | turbid, pale | pure | | | | |
| | yellow | | | | | |
| HH-327 | Turbid, pale | 10\hat{ }4 | 0.0432 | | | |
| | yellow | S/GEN | | | | |
| HH-328 | Clear, pale | NG | 0.0469 | | | |
| | yellow | | | | | |
| HH-329 | Clear, pale | >10\hat{ }5, | 0.0464 | E. coli | ND | X |
| | yellow | pure | | | | |
| HH-330 | Clear, pale | NG | 0.0137 | | | |
| | yellow | | | | | |
| HH-331 | Clear, pale | 10\hat{ }3, | 0.0409 | | | |
| | yellow | S/GEN | | | | |
| HH-332 | Clear, pale | NG | 0.0319 | | | |
| | yellow | | | | | |
| HH-333 | Clear, pale | NG | 0.0582 | | | |
| | yellow | | | | | |
| HH-334 | Clear, pale | NG | 0.0653 | | | |
| | yellow | | | | | |
| HH-335 | Clear, pale | 10\hat{ }3, | 0.0287 | | | |
| | yellow | S/GEN | | | | |
| HH-336 | Clear, pale | NG | 0.0322 | | | |
| | yellow | | | | | |
| HH-337 | Clear, pale | 10\hat{ }3, | 0.0416 | | | |
| | yellow | S/GEN | | | | |
| HH-338 | Clear, pale | NG | 0.0153 | | | |
| | yellow | | | | | |
| HH-339 | Clear, pale | >10\hat{ }5 | 0.0131 | Corynebacterium | | |
| | yellow | | | sp. | | |
| HH-340 | Slightly | 10\hat{ }3, | 0.0407 | | | |
| | turbid, pale | S/GEN | | | | |
| | yellow | | | | | |
| HH-341 | Turbid, pale | 10\hat{ }3, | 0.0743 | | | |
| | yellow | S/GEN | | | | |
| HH-342 | Slightly | 10\hat{ }5, | 0.0231 | | | |
| | turbid, pale | S/GEN | | | | |
| | yellow | | | | | |
| HH-343 | Clear, pale | >10\hat{ }5 | 0.0392 | E. coli | ND | X |
| | yellow | | | | | |
| HH-344 | Clear, pale | >10\hat{ }5, | 0.0323 | | | |
| | yellow | S/GEN | | | | |
| HH-345 | Clear, pale | NG | 0.0586 | | | |
| | yellow | | | | | |
| HH-346 | Clear, pale | 10\hat{ }4, | 0.0171 | E. coli | TEM-1 | X |
| | yellow | pure | | | | |
| HH-347 | Clear, pale | NG | 0.0232 | | | |
| | yellow | | | | | |
| HH-348 | Clear, pale | NG | 0.0183 | | | |
| | yellow | | | | | |
| HH-349 | Clear, pale | NG | 0.0447 | | | |
| | yellow | | | | | |
| HH-350 | Clear, pale | 10\hat{ }4 | 0.0417 | | | |
| | yellow | S/GEN | | | | |
| HH-351-1 | Clear, pale | 10\hat{ }4 | 0.6123 | E. hormaechei | presumed | X |
| | yellow | multiple | | | cAmpC: ND | |
| | | G− | | | for others | |
| HH-351-2 | | | | K. | SHV-148 | X |
| | | | | pneumoniae | | |
| HH-352 | Clear, pale | 10\hat{ }4 | 0.0785 | | | |
| | yellow | S/GEN | | | | |
| HH-353 | Clear, pale | >10\hat{ }5 | 0.0547 | E. coli | ND | X |
| | yellow | | | | | |
| HH-354 | Clear, pale | 10\hat{ }4 | 0.0107 | | | |
| | yellow | S/GEN | | | | |
| HH-355 | Clear, pale | 10\hat{ }4 | 0.0596 | | | |
| | yellow | S/GEN | | | | |
| HH-356 | Clear, pale | NG | 0.0500 | | | |
| | yellow | | | | | |
| HH-357 | Slightly | NG | 0.0279 | | | |
| | turbid, pale | | | | | |
| | yellow | | | | | |
| HH-358 | Slightly | >10\hat{ }5 | 0.0412 | E. coli | TEM-1 | X |
| | turbid, pale | | | | | |
| | yellow | | | | | |
| HH-359 | Clear, pale | >10\hat{ }5 | 0.0590 | P. mirabilis | ND | X |
| | yellow | | | | | |
| HH-360 | Clear, pale | 10\hat{ }5 | 0.0699 | | | |
| | yellow | S/GEN | | | | |
| HH-361 | Slightly | NG | 0.1812 | | | |
| | turbid, pale | | | | | |
| | yellow | | | | | |
| HH-362 | Clear, pale | 10\hat{ }4 | 0.0451 | | | |
| | yellow | S/GEN | | | | |
| HH-363 | Clear, pale | >10\hat{ }5 | 0.0564 | K. | SHV-100 | X |
| | yellow | | | pneumoniae | | |
| HH-364 | Clear, pale | 10\hat{ }4 | 0.0306 | | | |
| | yellow | S/GEN | | | | |
| HH-365 | Clear, pale | >10\hat{ }5, | 0.0343 | K. | SHV-61 | X |
| | yellow | pure | | pneumoniae | | |
| HH-366 | Clear, pale | 10\hat{ }4 | 0.0618 | C. freundii | CMY-41/112 | Negative |
| | yellow | | | | | |
| HH-367 | Slightly | >10\hat{ }5 | 0.0600 | | | |
| | turbid, pale | S/GEN | | | | |
| | yellow | | | | | |
| HH-368 | Slightly | 10\hat{ }3, | 0.0604 | | | |
| | turbid, pale | S/GEN | | | | |
| | yellow | | | | | |
| HH-369 | Clear, pale | 10\hat{ }4 | 0.0512 | | | |
| | yellow | S/GEN | | | | |
| HH-370 | Clear, pale | NG | 0.0646 | | | |
| | yellow | | | | | |
| HH-371 | Turbid, pale | 10\hat{ }3, | 0.0471 | | | |
| | yellow | S/GEN | | | | |
| HH-372-1 | Clear, pale | >10\hat{ }5 | 1.2620 | P. mirabilis | ND | X |
| | yellow | multiple | | | | |
| | | G− | | | | |
| HH-372-2 | | | | P. | presumed | Negative |
| | | | | aeruginosa | cAmpC; ND | |
| | | | | | for others | |
| HH-373 | Clear, pale | >10\hat{ }5 | 0.0552 | E. coli | ND | X |
| | yellow | | | | | |
| HH-374 | Clear, pale | 10\hat{ }3, | 0.0813 | | | |
| | yellow | S/GEN | | | | |
| HH-375 | Slightly | >10\hat{ }5, | 0.0713 | E. coli | TEM-1 | X |
| | turbid, pale | pure | | | | |
| | yellow | | | | | |
| HH-376 | Clear, pale | >10\hat{ }5 | 0.0409 | P. mirabilis | ND | X |
| | yellow | | | | | |
| HH-377 | Clear, pale | >10\hat{ }5 | 0.0000 | E. coli | ND | X |
| | yellow | | | | | |
| HH-378 | Clear, pale | NG | 0.0691 | | | |
| | yellow | | | | | |
| HH-379 | Turbid, pale | 10\hat{ }4 | 0.0841 | | | |
| | yellow | S/GEN | | | | |
| HH-380 | Clear, pale | NG | 0.0048 | | | |
| | yellow | | | | | |
| HH-381 | Clear, pale | 10\hat{ }4 | 0.0761 | | | |
| | yellow | S/GEN | | | | |
| HH-382 | Clear, pale | 10\hat{ }3, | 0.0606 | | | |
| | yellow | S/GEN | | | | |
| HH-383 | Clear, pale | NG | 0.0673 | | | |
| | yellow | | | | | |
| HH-384 | Turbid, pale | >10\hat{ }5, | 0.0000 | E. coli | ND | X |
| | yellow | pure | | | | |
| HH-385 | Clear, bright | NG | 0.0634 | | | |
| | orange | | | | | |
| HH-386 | Clear, pale | NG | 0.0769 | | | |
| | yellow | | | | | |
| HH-387 | Clear, pale | 10\hat{ }5 | 0.0663 | | | |
| | yellow | S/GEN | | | | |
| HH-388 | Clear, pale | 10\hat{ }4 | 0.0969 | | | |
| | yellow | S/GEN | | | | |
| HH-389 | Clear, pale | 10\hat{ }5 | 0.0667 | | | |
| | yellow | S/GEN | | | | |
| HH-390 | Clear, pale | 10\hat{ }3 | 0.1243 | | | |
| | yellow | S/GEN | | | | |
| HH-391 | Clear, pale | >10\hat{ }5, | 0.1181 | E. coli | ND | X |
| | yellow | pure | | | | |
| HH-392 | Clear, pale | NG | 0.0557 | | | |
| | yellow | | | | | |
| HH-393 | Clear, pale | NG | 0.0905 | | | |
| | yellow | | | | | |
| HH-394 | Clear, pale | NG | 0.1337 | | | |
| | yellow | | | | | |
| HH-395 | Slightly | 10\hat{ }4 | 0.0730 | | | |
| | turbid, pale | S/GEN | | | | |
| | yellow | | | | | |
| HH-396 | Clear, pale | 10\hat{ }3, | 0.0696 | | | |
| | yellow | pure | | | | |
| HH-397 | Clear, pale | 10\hat{ }3 | 0.1248 | | | |
| | yellow | S/GEN | | | | |
| HH-398 | Clear, pale | 10\hat{ }3 | 0.0736 | | | |
| | yellow | S/GEN | | | | |
| HH-399 | Clear, pale | 10\hat{ }3 | 0.0681 | | | |
| | yellow | S/GEN | | | | |
| HH-400 | Clear, pale | NG | 0.0849 | | | |
| | yellow | | | | | |
| HH-401 | Clear, pale | 10\hat{ }3 | 0.0829 | | | |
| | yellow | S/GEN | | | | |
| HH-402 | Slightly | 10\hat{ }4 | 0.0931 | | | |
| | turbid, pale | S/GEN | | | | |
| | yellow | | | | | |
| HH-403 | Clear, pale | 10\hat{ }3 | 0.0928 | | | |
| | yellow | S/GEN | | | | |
| HH-404 | Clear, pale | 10\hat{ }4 | 0.1005 | | | |
| | yellow | S/GEN | | | | |
| HH-405 | Clear, pale | 10\hat{ }4 | 0.1127 | | | |
| | yellow | S/GEN | | | | |
| HH-406 | Clear, pale | NG | 0.0941 | | | |
| | yellow | | | | | |
| HH-407 | Turbid, pale | >10\hat{ }5 | 0.1195 | E. coli | ND | X |
| | yellow | | | | | |
| HH-408 | Clear, pale | 10\hat{ }4 | 0.0890 | | | |
| | yellow | S/GEN | | | | |
| HH-409 | Turbid, pale | >10\hat{ }5 | 0.8693 | P. mirabilis | TEM-1, | X |
| | yellow | | | | DHA-9? | |
| HH-410 | Slightly | 10\hat{ }4 | 0.0456 | E. faecalis | X | X |
| | turbid, pale | | | | | |
| | yellow | | | | | |
| HH-411 | Clear, pale | 10\hat{ }4 | 0.0620 | | | |
| | yellow | S/GEN | | | | |
| HH-412 | Clear, pale | 10\hat{ }3 | 0.0618 | | | |
| | yellow | S/GEN | | | | |
| HH-413 | Clear, pale | NG | 0.0422 | | | |
| | yellow | | | | | |
| HH-414 | Clear, pale | 10\hat{ }4 | 0.0766 | | | |
| | yellow | S/GEN | | | | |
| HH-415 | Clear, pale | >10\hat{ }5 | 1.6040 | E. coli | OXA-1, | Positive |
| | yellow | | | | CTX-M-15 | |
| HH-416 | Clear, pale | 10\hat{ }3 | 0.0953 | | | |
| | yellow | S/GEN | | | | |
| HH-417 | Clear, pale | 10\hat{ }4 | 0.0721 | | | |
| | yellow | S/GEN | | | | |
| HH-418 | Clear, pale | 10\hat{ }3 | 0.0889 | | | |
| | yellow | S/GEN | | | | |
| HH-419 | Clear, pale | >10\hat{ }5, | 0.0490 | E. coli | ND | X |
| | yellow | pure | | | | |
| HH-420 | Slightly | 10\hat{ }3 | 0.0990 | | | |
| | turbid, pale | S/GEN | | | | |
| | yellow | | | | | |
| HH-421 | Clear, pale | 10\hat{ }3 | 0.0594 | | | |
| | yellow | S/GEN | | | | |
| HH-422 | Clear, pale | 10\hat{ }3 | 0.0724 | | | |
| | yellow | S/GEN | | | | |
| HH-423 | Clear, pale | NG | 0.0469 | | | |
| | yellow | | | | | |
| HH-424 | Slightly | 10\hat{ }4 | 0.0690 | E. coli | TEM-1 | X |
| | turbid, pale | | | | | |
| | yellow | | | | | |
| HH-425 | Clear, pale | 10\hat{ }4 | 0.0562 | | | |
| | yellow | S/GEN | | | | |
| HH-426 | Clear, pale | 10\hat{ }4 | 0.0580 | | | |
| | yellow | S/GEN | | | | |
| HH-427 | Clear, pale | 10\hat{ }4 | 0.0553 | | | |
| | yellow | S/GEN | | | | |
| HH-428 | Clear, pale | 10\hat{ }3 | 0.0705 | | | |
| | yellow | S/GEN | | | | |
| HH-429 | Slightly | 10\hat{ }4-5 | 0.0152 | Group B | | |
| | turbid, pale | | | Streptococcus | | |
| | yellow | | | | | |
| HH-430 | Clear, pale | 10\hat{ }4-5 | 0.0895 | E. coli | TEM-1 | X |
| | yellow | | | | | |
| HH-431 | Clear, pale | 10\hat{ }3 | 0.0939 | | | |
| | yellow | S/GEN | | | | |
| HH-432 | Clear, pale | NG | 0.0621 | | | |
| | yellow | | | | | |
| HH-433 | Clear, pale | 10\hat{ }5 | 0.0765 | | | |
| | yellow | S/GEN | | | | |
| HH-434-1 | Slightly | >10\hat{ }5 | 0.5443 | K. | SHV-60 | X |
| | turbid, red | multiple | | pneumoniae | | |
| | | G− | | | | |
| HH-434-2 | | | | P. mirabilis | TEM-1, | Positive |
| | | | | | CTX-M14 | |
| HH-435 | Turbid, pale | >10\hat{ }5 | 0.0890 | | | |
| | yellow | S/GEN | | | | |
| HH-436 | Turbid, pale | NG | 0.0627 | | | |
| | yellow | | | | | |
| HH-437 | Turbid, pale | 10\hat{ }3 | 0.0606 | | | |
| | yellow | S/GEN | | | | |
| HH-438 | Clear, bright | 10\hat{ }4 | 0.0576 | | | |
| | orange | S/GEN | | | | |
| HH-439 | Clear, pale | NG | 0.0525 | | | |
| | yellow | | | | | |
| HH-440 | Slightly | >10\hat{ }5 | 0.1058 | Staphylococcus | | |
| | turbid, pale | | | sp. | | |
| | yellow | | | | | |
| HH-441 | Clear, pale | 10\hat{ }3 | 0.0729 | | | |
| | yellow | S/GEN | | | | |
| HH-442 | Clear, bright | NG | 0.0000 | | | |
| | orange | | | | | |
| HH-443 | Clear, pale | 10\hat{ }4 | 0.0789 | | | |
| | yellow | S/GEN | | | | |
| HH-444 | Clear, pale | NG | 0.0301 | | | |
| | yellow | | | | | |
| HH-445 | Turbid, | NG | 0.0000 | | | |
| | bright orange | | | | | |
| HH-446 | Slightly | >10\hat{ }5, | 0.6987 | E. coli | TEM-1 | X |
| | turbid, pale | pure | | | | |
| | yellow | | | | | |
| HH-447 | Turbid, | NG | 0.1019 | | | |
| | bright orange | | | | | |
| HH-448 | Clear, bright | 10\hat{ }3 | 0.0563 | | | |
| | orange | S/GEN | | | | |
| HH-449 | Clear, pale | NG | 0.0623 | | | |
| | yellow | | | | | |
| HH-450-1 | Slightly | >10\hat{ }5 | 0.1053 | K. | SHV-83 | X |
| | turbid, pale | multiple | | pneumoniae | | |
| | yellow | G− | | | | |
| HH-450-2 | | | | P. mirabilis | ND | X |
| HH-451 | Clear, pale | NG | 0.0683 | | | |
| | yellow | | | | | |
| HH-452-1 | Slightly | >10\hat{ }5 | 0.0992 | K. | SHV-83/187 | X |
| | turbid, pale | multiple | | pneumoniae | | |
| | yellow | G− | | | | |
| HH-452-2 | | | | E. coli | ND | X |
| HH-453 | Turbid, | NG | 0.0156 | | | |
| | bright orange | | | | | |
| HH-454 | Turbid, pale | 10\hat{ }3 | 0.0230 | | | |
| | yellow | S/GEN | | | | |
| HH-455 | *None | >10\hat{ }5 | 0.0358 | Alpha- | | |
| | recorded* | | | hemolytic | | |
| | | | | Viridans | | |
| | | | | Streptococcus | | |
| HH-456 | Clear, pale | 10\hat{ }4 | 0.0000 | | | |
| | yellow | S/GEN | | | | |
| HH-457 | Turbid, pale | >10\hat{ }5, | 0.0402 | E. coli | ND | X |
| | yellow | pure | | | | |
| HH-458 | Clear, pale | >10\hat{ }5 | 0.0267 | E. faecalis | X | X |
| | yellow | | | | | |
| HH-459 | Clear, pale | NG | 0.0525 | | | |
| | yellow | | | | | |
| HH-460 | Clear, pale | 10\hat{ }3 | 0.0606 | | | |
| | yellow | S/GEN | | | | |
| HH-461 | Clear, pale | NG | 0.0140 | | | |
| | yellow | | | | | |
| HH-462 | Slightly | 10\hat{ }4-5 | 0.0230 | | | |
| | turbid, pale | S/GEN | | | | |
| | yellow | | | | | |
| HH-463 | Clear, pale | NG | 0.0332 | | | |
| | yellow | | | | | |
| HH-464 | Turbid, pale | NG | 0.0549 | | | |
| | yellow | | | | | |
| HH-465 | Slightly | >10\hat{ }5, | 1.4840 | E. coli | OXA-1, | Positive |
| | turbid, pale | pure | | | CTX-M-15 | |
| | yellow | | | | | |
| HH-466 | Clear, bright | NG | 0.0281 | | | |
| | orange | | | | | |
| HH-467 | Clear, pale | 10\hat{ }4 | 0.0407 | | | |
| | yellow | S/GEN | | | | |
| HH-468 | Clear, pale | >10\hat{ }5 | 0.0187 | Group B | | |
| | yellow | | | Streptococcus | | |
| HH-469 | Clear, pale | 10\hat{ }4-5, | 0.0468 | | | |
| | yellow | S/GEN | | | | |
| HH-470 | Clear, pale | >10\hat{ }5, | 1.9742 | E. coli | CTX-M-15 | Positive |
| | yellow | pure | | | | |
| HH-471 | Clear, pale | NG | 0.0445 | | | |
| | yellow | | | | | |
| HH-472 | Clear, bright | >10\hat{ }5 | 0.0246 | Group B | | |
| | orange | | | Streptococcus | | |
| HH-473 | Turbid, pale | 10\hat{ }3 | 0.0271 | | | |
| | yellow | S/GEN | | | | |
| HH-474 | Slightly | >10\hat{ }5 | 0.0648 | E. coli | TEM-1 | X |
| | turbid, pale | | | | | |
| | yellow | | | | | |
| HH-475 | Clear, pale | 10\hat{ }4 | 0.0322 | | | |
| | yellow | S/GEN | | | | |
| HH-476 | Clear, pale | 10\hat{ }4 | 0.0261 | E. coli | TEM-1 | X |
| | yellow | S/GEN | | | | |

^{a}If more than one organism was isolated from the urine sample, the urine sample no. is listed more than once to indicate the number of species identified at significant CFU/mL (ex: HH-098-1, HH-098-2, HH-098-3).
^{b}Isolates with any β-lactam resistance (resistant at least to ampicillin) were tested for carriage of β-lactamase genes. The chromosomal AmpC of E. coli was not screened for by PCR, and of the K. pneumoniae chromosomal β-lactamases, only SHV was properly screened for (though LEN was sometimes detected with SHV primers). The cAmpCs from other Gram-negative bacterial species were also not tested for, but were assumed to be present.
^{c}The Kirby-Bauer disk-diffusion method of ESBL confirmatory testing (according to CLSI) was used.

A combination of microbiology and molecular biology results were used as the reference by which DETECT was compared: (a) a “reference standard positive” was defined as a microbiologically-defined UTI sample containing a GNB isolate with a positive ESBL confirmatory test (CLSI disk-diffusion method) that was also positive for a CTX-M gene (by PCR and amplicon sequencing) [N=11 samples]; (b) a “reference standard negative” was defined as any sample not satisfying the reference standard positive criteria [N=460 samples]. A ROC curve was constructed to establish a threshold value for a positive DETECT Score, and optimize DETECT assay specifications. This resulted in an AUC of 0.937 (95% CI: 0.828 to 1.047). A cutoff value of 0.2588 was selected, which afforded a dually high sensitivity (91%) and specificity (98%) for DETECT (see FIG. 5B).

Only twelve urine samples generated DETECT results that were considered incorrect. When possible, bacteria isolated from these urine samples were retested with DETECT as individual clinical isolates, to further understand the discordance between expected and observed DETECT results. One “reference standard positive” urine sample tested false-negative by DETECT; the CTX-M-15-producing K. pneumoniae isolated from this sample generated a correct positive DETECT result (see TABLE 7).

TABLE 7
Bacterial isolates from urine samples generating discrepant
results, tested with DETECT.

| | DETECT | | | | | DETECT | |
|---|---|---|---|---|---|---|---|
| | Score | | | | β-lactamase | Score | |
| Urine No. | (urine) | Int.^{a} | CFU/mL^{b} | Organism ID | genes^{c} | (isolate) | Int.^{e} |
| HH-001 | 0.3177 | FP | >10⁵, | E. coli | TEM-1 | 0.1595 | Neg |
| | | | pure | | | | |
| HH-003 | 0.4551 | FP | >10⁵, | E. coli | TEM-1 | 0.1226 | Neg |
| | | | pure | | | | |
| HH-068 | 0.5805 | FP | >10⁵ | E. coli | TEM-1 | 0.2047 | Neg |
| HH-128 | 0.2914 | FP | >10⁵ | E. coli | TEM-1 | 0.1682 | Neg |
| | | | | K. pneumoniae | SHV-11 | 0.843 | Neg |
| | | | | P. mirabilis | ND | 0.122 | Neg |
| HH-131 | 0.2724 | FP | >10⁵ | E. coli | TEM-1 | 0.1596 | Neg |
| HH-165 | 0.2608 | FP | >10⁵ | X | X | X | X |
| | | | S/GEN | | | | |
| HH-236 | X | Error | >10⁵ | K. pneumoniae | SHV-148 | 0.1155 | Neg |
| | | | | E. coli | TEM-10 | | |
| | | | | | (ESBL) | | |
| HH-261 | 0.0400 | FN | 10^{4to 5}, | K. pneumoniae | SHV-28, | 0.3192 | Pos |
| | | | pure | | OXA-1, | 0.4519 | Pos |
| | | | | | CTX-M-15 | | |
| HH-297 | 0.8374 | FP | >10⁵, | P. rettgeri | Presumed | 0.1299 | Neg |
| | | | pure | | cAmpC | | |
| HH-351 | 0.6123 | FP | 10⁴ | E. hormaechei | Presumed | 0.2012 | Neg |
| | | | | | cAmpC | | |
| | | | | K. pneumoniae | SHV-148 | 0.1228 | Neg |
| HH-372 | 1.2620 | FP | >10⁵ | P. mirabilis | ND | 0.1401 | Neg |
| | | | | P. aeruginosa | Presumed | 0.1302 | Neg |
| | | | | | cAMPC | | |
| HH-409 | 0.8693 | FP | >10⁵ | P. mirabilis | TEM-1, | 0.173 | Neg |
| | | | | | DHA-9^{d} | | |
| HH-446 | 0.6987 | FP | >10⁵, | E. coli | TEM-1 | 0.1988 | Neg |
| | | | pure | | | | |
| HH-366 | 0.0618 | TN, | 10⁴ | C. freundii | cAmpC | 1.9926 | Pos |
| | | (EP) | | | (CMY-41/112) | | |

^{a}Int., interpretation of DETECT result with urine (threshold = 0.2588); FP, false-positive; Error, DETECT Score could not be generated due to an oversaturation of signal at 30 min; FN, false-negative; EP, expected positive (even though the urine sample generated a “correct” result, it was expected to produce a FP result due to CMY β-lactamase content and 3^{rd}-generation cephalosporin resistance).
^{b}“Pure” indicates the urine sample yielded a pure culture of the indicated organism. When “pure” is not indicated, the sample also contained insignificant CFU of skin/urogenital flora. G−, Gram-negative bacteria.
^{c}Presumed cAmpC indicates the species is known to contain cAmpCs. Due to their intrinsic nature, these enzymes were not tested for by PCR but were assumed to be present. ND, none detected.
^{d}The P. mirabilis isolate was found to be DHA-9-positive by PCR (pArnpC). though it lacked a (β-lactam-resistance phenotype associated with plasmid-mediated DHA genes (i.e. third-generation cephalosporin resistance).
^{e}Interpretation of DETECT result with clinical isolates (threshold = 0.2806).

Eleven “reference standard negative” urine samples tested false-positive by DETECT. Bacteria cultured from 10 of these samples generated the following correct negative DETECT results (note that some samples grew more than one organism in significant numbers, so all isolates were tested): six TEM-1-producing E. coli tested negative; two SHV-producing K. pneumoniae tested negative; two β-lactam-susceptible P. mirabilis and one TEM-1/DHA-9-positive P. mirabilis tested negative; three cAmpC-producing GNB tested negative. One “reference standard negative” urine sample was not able to be retested since it had not been considered by the clinical laboratory to be a UTI (10⁵ CFU/mL mixed skin/genitourinary flora), and the mixed bacteria cultured from this urine sample had not been saved. A DETECT Score could not be determined for one urine sample (error) because the sample generated an A_{405 nm} signal at 30 min that exceeded the spectrophotometer's detection range (A_{405 nm}>4.0). Surprisingly, the TEM-10-producing E. coli isolated from this sample generated a positive DETECT result. Interestingly, one DETECT-negative urine sample grew a 3′-generation cephalosporin-resistant C. freundii (produces a CMY type cAmpC); based on the CMY genotype and resistance phenotype of this organism, we would have expected this urine sample to generate a positive result in DETECT. Therefore, we tested the C. freundii isolate with DETECT and found that it generated a positive result (demonstrating concordance with previous CMY-producing isolate experiments).

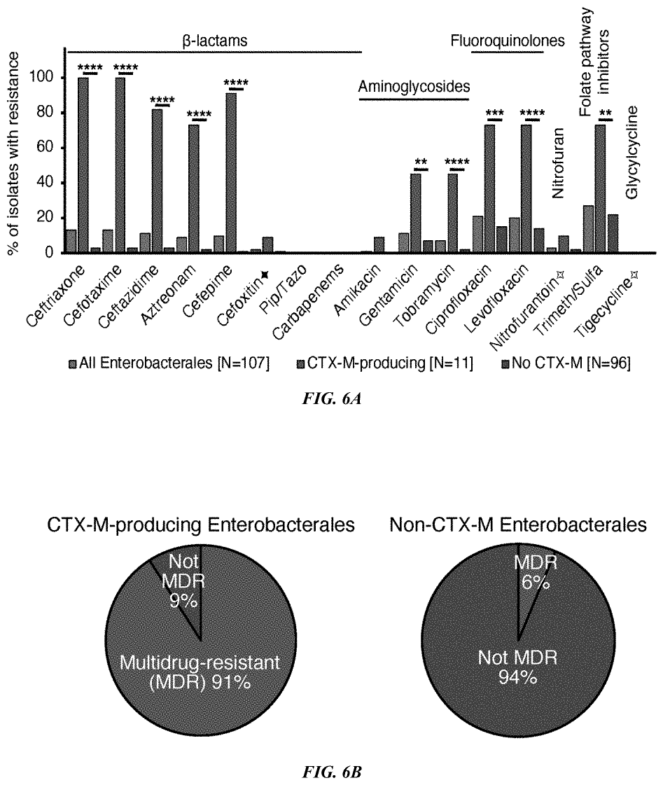

CTX-M-producing bacteria causing UTI have limited antibiotic treatment options. The CTX-M-producing isolates identified in this study included E. coli (8 isolates), K. pneumoniae (2 isolates), and P. mirabilis (1 isolate)—all members of the family Enterobacteriaceae, and the only family containing CTX-M-producing bacteria in this study. The Enterobacteriaceae isolates were further evaluated to determine the antimicrobial resistance profile across CTX-M-producing bacteria and bacteria lacking CTX-Ms in this study (see FIG. 6A). Most 3^{rd}-generation cephalosporin resistance (ceftriaxone, cefotaxime, ceftazidime) could be attributed to CTX-M-producing bacteria. Three exceptions were a TEM-10 ESBL-producing E. coli, an SHV-9/12 ESBL-producing K. pneumoniae, and a cAmpC CMY-41/112-producing C. freundii. Likewise, resistance to aztreonam (monobactam) and cefepime (4^{th}-generation cephalosporin) were mainly due to CTX-M-producing bacteria. Excluding intrinsic resistance from cAmpC-producing Enterobacteriaceae, resistance to cefoxitin was rare; piperacillin/tazobactam resistance and carbapenem resistance were not detected in the isolates. Therefore, by correctly identifying 10 (91%) of 11 CTX-M-positive urine samples, DETECT identified 71% (10 of 14) of the expanded-spectrum cephalosporin resistance found in this study.

Of the aminoglycosides, amikacin resistance occurred in only one CTX-M-producing E. coli. In contrast, gentamicin resistance was identified in 5 (45%) CTX-M-producing bacteria and 7 (7%) bacteria lacking CTX-Ms (P<0.01), while tobramycin resistance was identified in 5 (45%) CTX-M-producing bacteria and 2 (2%) bacteria lacking CTX-Ms (P<0.0001). Fluoroquinolone and trimethoprim/sulfamethoxazole resistance was more prevalent across all isolates; however, resistance to agents in these classes was still more likely to occur in CTX-M-producing bacteria. Ciprofloxacin resistance was identified in 8 (73%) CTX-M-producing bacteria and 14 (15%) bacteria lacking CTX-Ms (P=0.0001); similarly, levofloxacin resistance was identified in 8 (73%) CTX-M-producing bacteria and 13 (14%) bacteria lacking CTX-Ms (P<0.0001). Additionally, trimethoprim/sulfamethoxazole resistance was identified in 8 (73%) CTX-M-producing bacteria and 21 (22%) bacteria lacking CTX-Ms (P<0.01). Excluding intrinsic resistance (P. mirabilis and P. rettgeri), nitrofurantoin resistance was rare; it was identified in 1 (10%) CTX-M-producing bacteria and 2 (2%) bacteria lacking CTX-Ms. Tigecycline has been considered for the treatment of UTIs caused by GNB with limited treatment options (including ESBL-EK). Excluding intrinsic resistance (P. mirabilis and P. rettgeri), no tigecycline-resistant isolates were identified.

Multidrug resistance (MDR) is typically defined as resistance to at least one agent in three or more classes of antimicrobial agents, excluding intrinsic resistance. Patients with MDR infections are less likely to receive concordant (by AST results) empiric treatment, because MDR bacteria are resistant to multiple potential treatment choices. CTX-M-producing bacteria were more likely to be MDR than other GNB causing UTI; 10 (91%) CTX-M-producing bacteria compared to six (6%) non-CTX-M bacteria (FIG. 6B) were MDR (P<0.0001). The positive predictive value for CTX-M-positive Enterobacteriaceae being MDR was 90.9% (CI: 57.8% to 98.6%), and the negative predictive value was 93.7% (CI: 88.8% to 96.6%). DETECT identified nine (90%) of 10 UTIs caused by MDR CTX-M-producing GNB.

It will be understood that various modifications may be made without departing from the spirit and scope of this disclosure. Accordingly, other embodiments are within the scope of the following claims.

What is claimed is:

1. A compound having the structure of Formula I or Formula II: 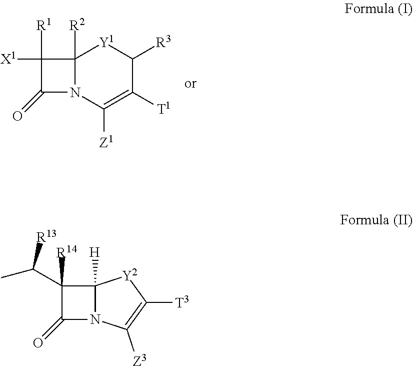
or a salt, stereoisomer, tautomer, polymorph, or solvate thereof, wherein:
T¹ is a benzenethiol containing group or Z², wherein if T¹ is Z², then Z¹ is T²;
Z¹ is a carboxylate, a carbonyl, an ester, an amide, a sulfone, a sulfonamide, a sulfonyl, —S(O)₂OH or T², wherein if Z¹ is T², then T¹ is Z²;
T² is a benzenethiol containing group;
T³ is a benzenethiol containing group;
Z² is a carboxylate, a carbonyl, an ester, an amide, a sulfone, a sulfonamide, a sulfonyl, or —S(O)₂OH;
Z³ is a carboxylate, a carbonyl, an ester, an amide, a sulfone, a sulfonamide, a sulfonyl, or —S(O)₂OH;
X¹ is 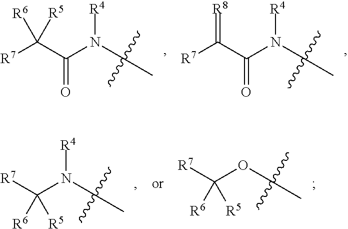
Y¹ is 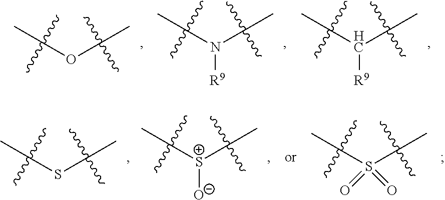
Y² is 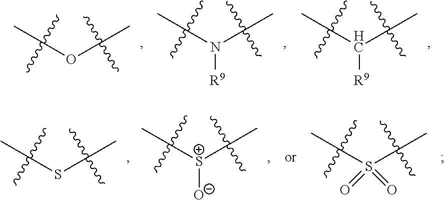
R¹-R⁶, R⁹-R¹¹, R¹³ and R¹⁴ are each independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle;
R⁷ is an optionally substituted (C₅-C₇) cycloalkyl, optionally substituted aryl, optionally substituted benzyl, or optionally substituted heterocycle; and
R⁸ is 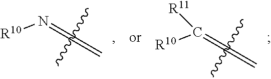
with the proviso that the compound does not have the structure of:

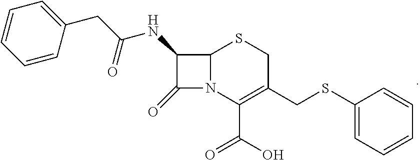 [SMILES: O=C(Cc1ccccc1)N[C@@H]1C(=O)N2C(C(=O)O)=C(CSc3ccccc3)CSC12]

2. The compound of claim 1, wherein T¹ or T² is a benzenethiol group selected from the group consisting of: 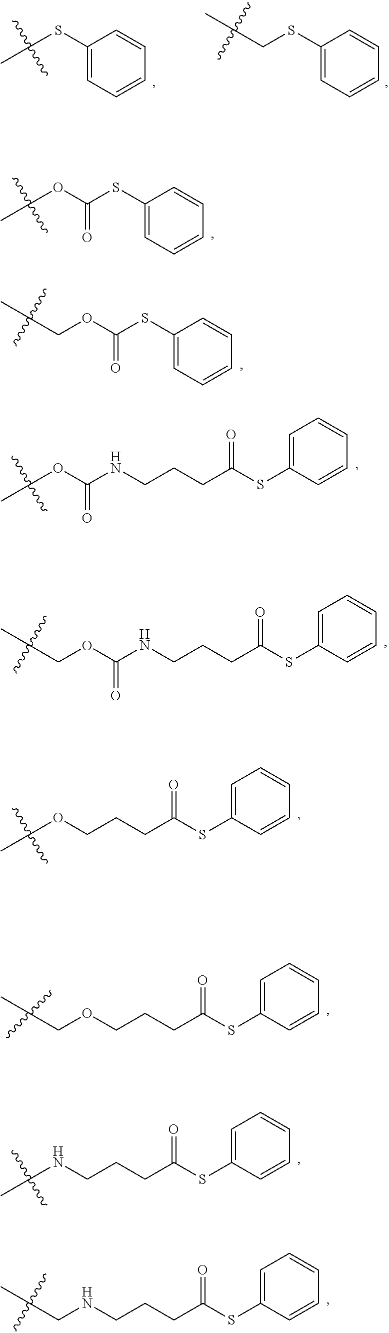 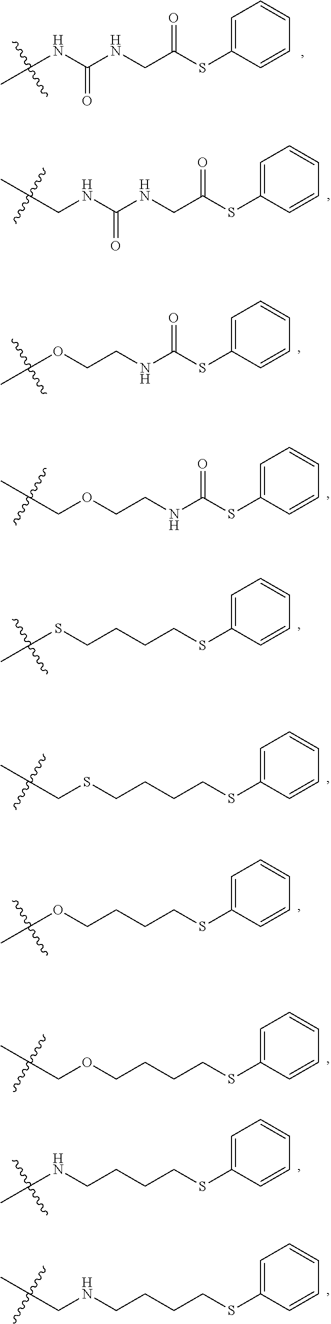 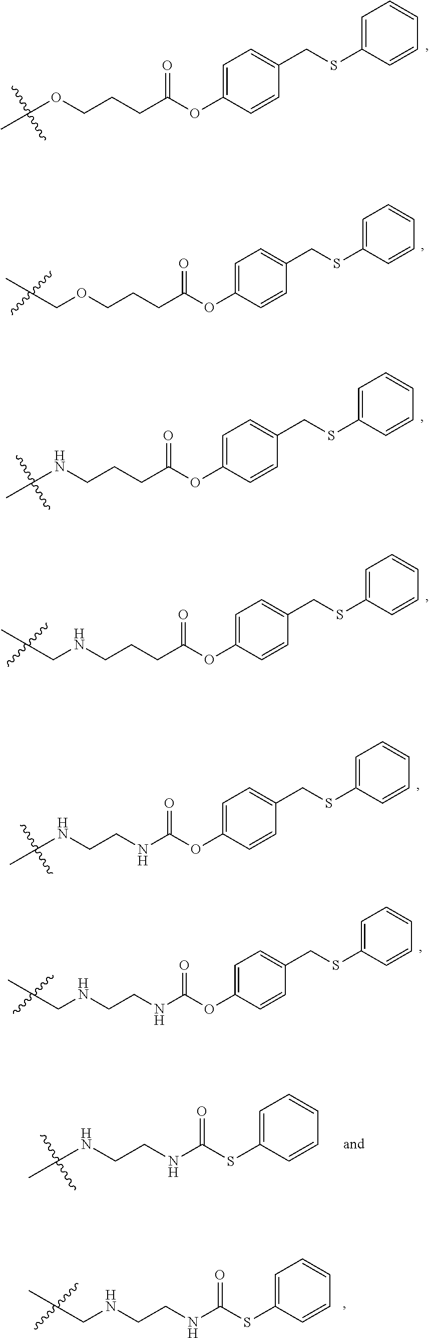
and/or wherein R⁷ is selected from the group consisting of:

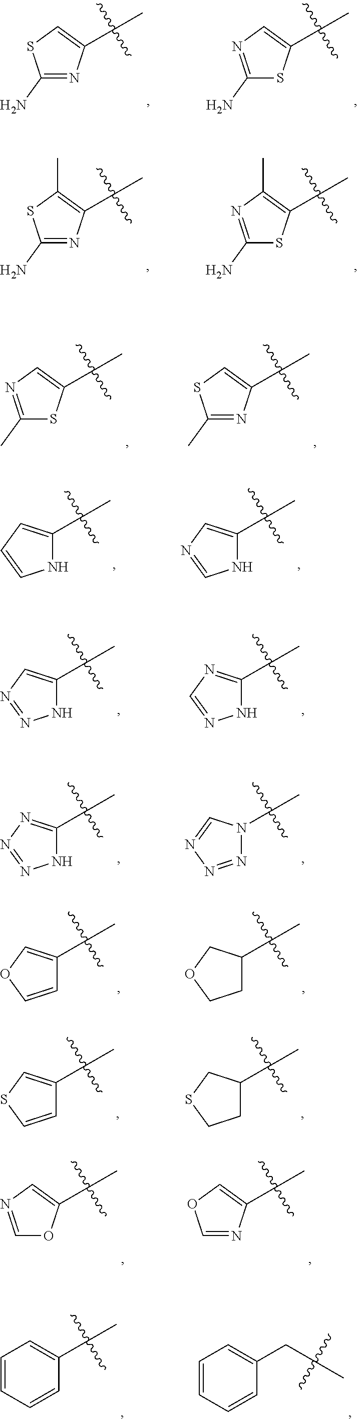

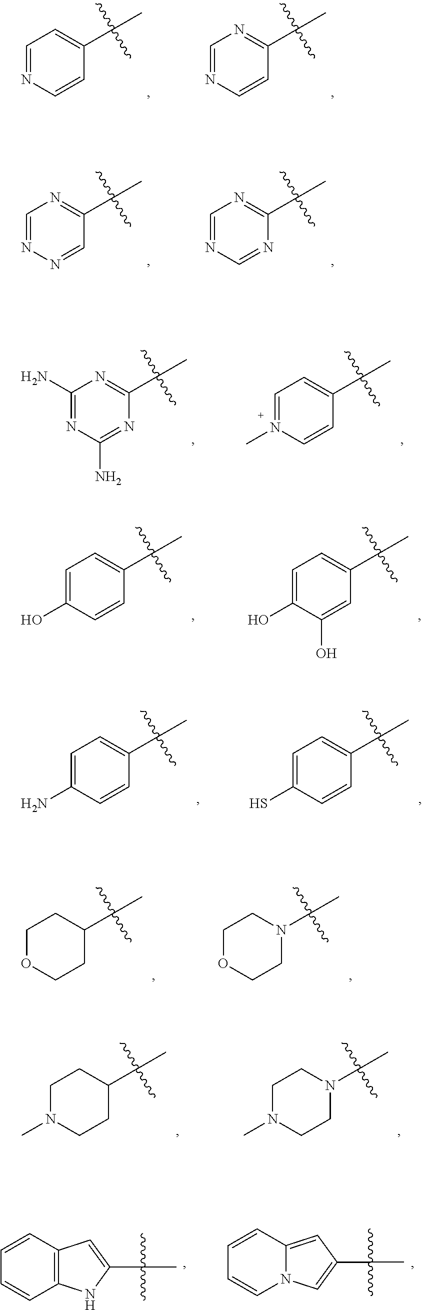

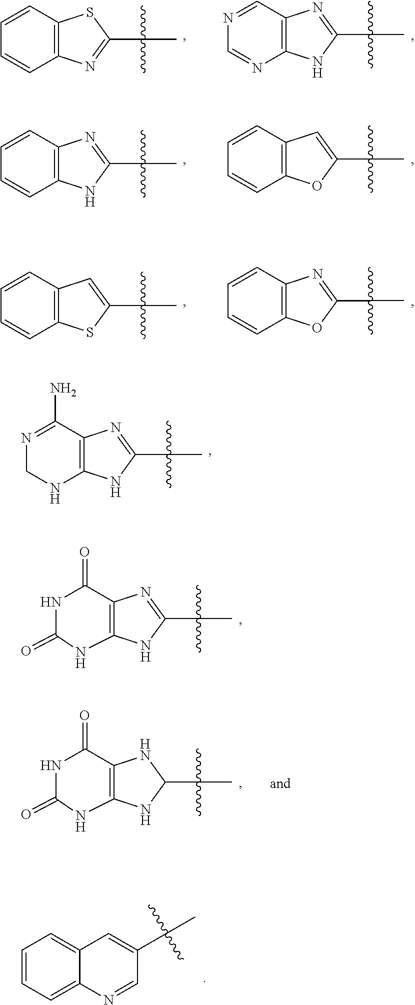

3. The compound of claim 1, wherein the compound has a structure of Formula I(a): 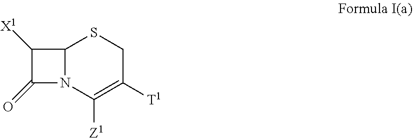
or a salt, stereoisomer, tautomer, polymorph, or solvate thereof, wherein:
T¹ is a benzenethiol containing group or Z², wherein if T¹ is Z², then Z¹ is T²;
Z¹ is a carboxylate, a carbonyl, an ester, an amide, a sulfone, a sulfonamide, a sulfonyl, —S(O)₂OH or T², wherein if Z¹ is T², then T¹ is Z²;
T² is a benzenethiol containing group;
Z² is a carboxylate, a carbonyl, an ester, an amide, a sulfone, a sulfonamide, a sulfonyl, or —S(O)₂OH;
X¹ is 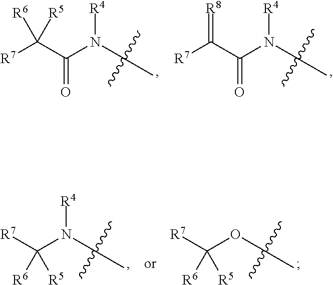
R⁴, R⁵, and R¹⁰ are independently an H or a (C₁-C₆)alkyl;
R⁶ is an H, or an amine;
R⁷ is an optionally substituted (C₅-C₇) cycloalkyl, optionally substituted aryl, optionally substituted benzyl, or optionally substituted heterocycle;
R⁸ is 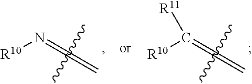
and
R⁹ is a hydroxyl or an (C₁-C₃)alkoxy.

4. The compound of claim 1, wherein the compound has the structure of Formula I(b): 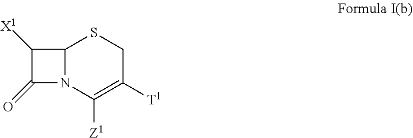
or a salt, stereoisomer, tautomer, polymorph, or solvate thereof, wherein:
T¹ a benzenethiol containing group selected from the group consisting 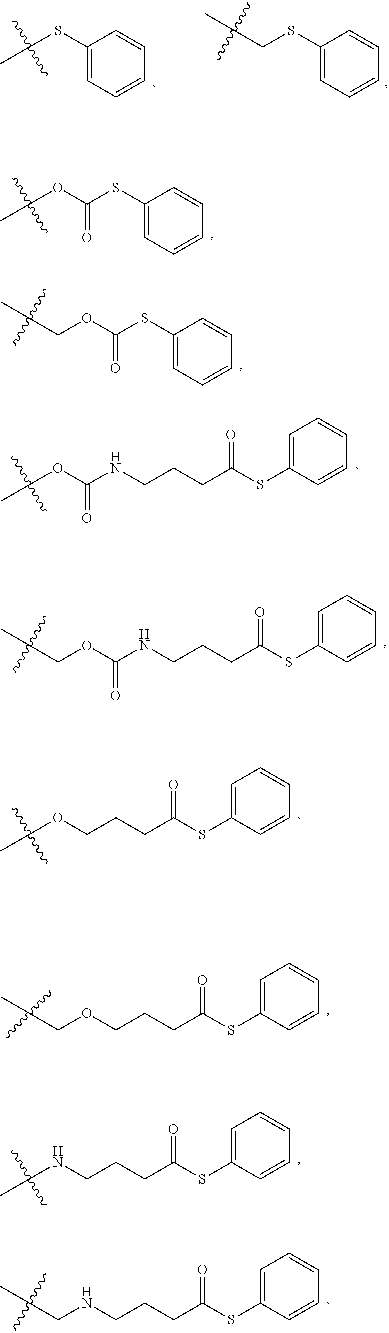 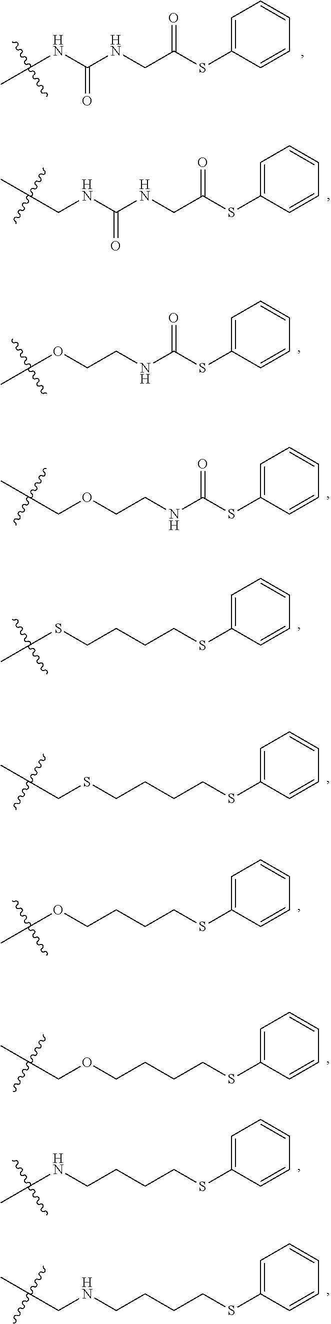 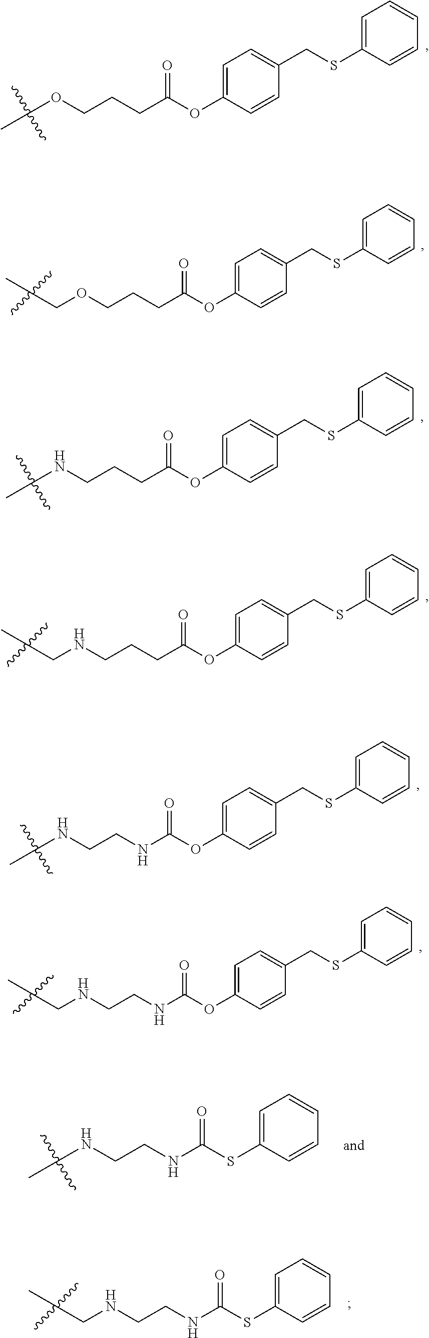
Z¹ is a carboxylate, a carbonyl, an ester, an amide, a sulfone, a sulfonamide, a sulfonyl, —S(O)₂OH or T²;
X¹ is 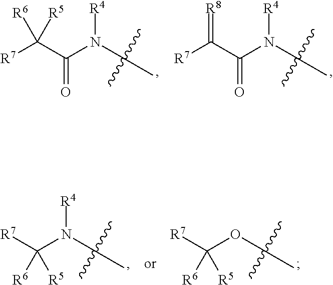
R⁴, R⁵, and R¹⁰ are independently an H or a (C₁-C₆)alkyl;
R⁶ is an H, or an amine;
R⁷ is an optionally substituted aryl, optionally substituted benzyl, or optionally substituted heterocycle;
R⁸ is 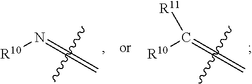
and
R⁹ is a hydroxyl or an (C₁-C₃)alkoxy.

5. The compound of claim 1, wherein the compound has the structure of Formula I(c): 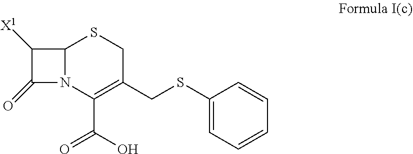
X¹ is 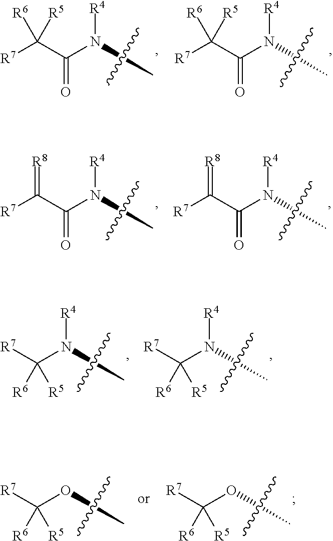
R⁴, R⁵, and R¹⁰ are independently an H or a (C₁-C₆)alkyl;
R⁶ is an H, or an amine;
R⁷ selected from the group consisting of: 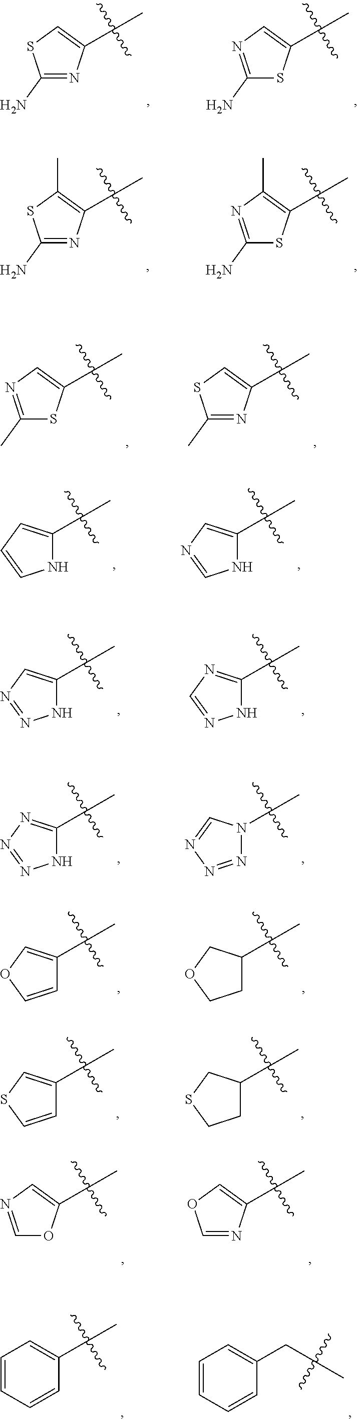 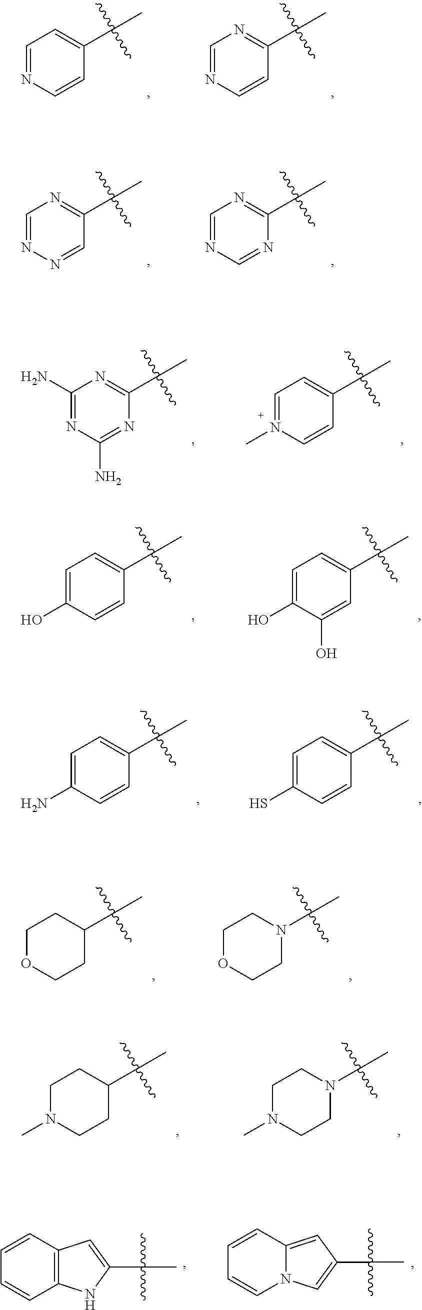 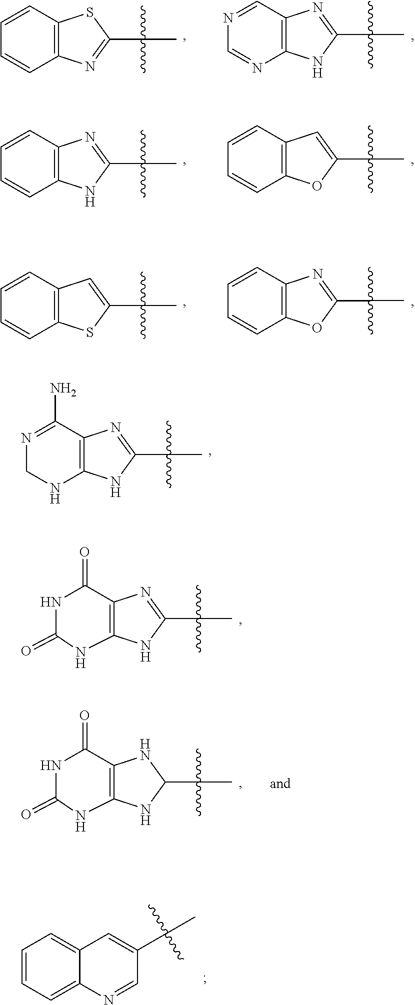
R⁸ is 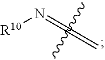
and
R⁹ is

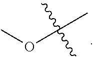 [SMILES: *OC]

6. The compound of claim 1, wherein the compound is selected from the group consisting of: 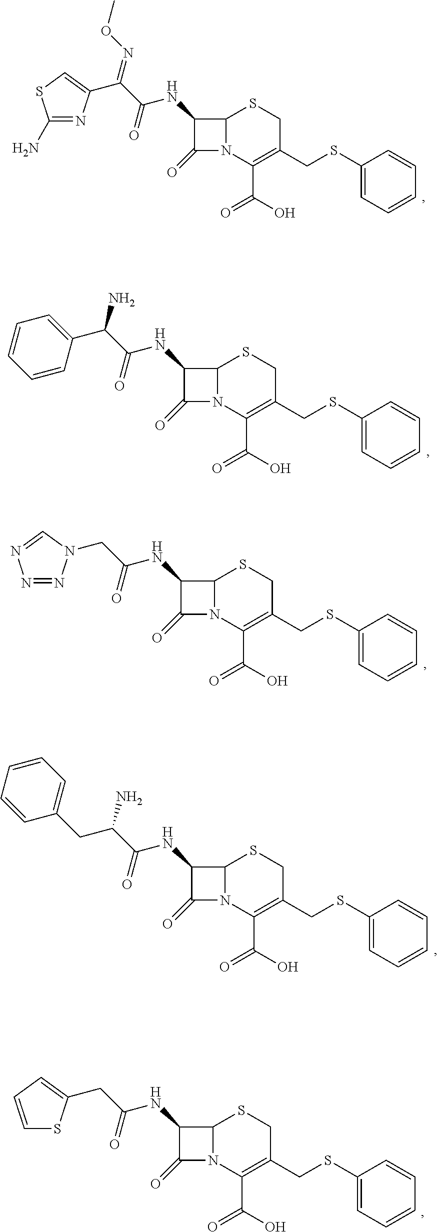 [SMILES: CO/N=C(/C(=O)N[C@@H]1C(=O)N2C(C(=O)O)=C(CSc3ccccc3)CSC12)c1csc(N)n1 ; N[C@@H](C(=O)N[C@@H]1C(=O)N2C(C(=O)O)=C(CSc3ccccc3)CSC12)c1ccccc1 ; O=C(Cn1cnnn1)N[C@@H]1C(=O)N2C(C(=O)O)=C(CSc3ccccc3)CSC12 ; N[C@@H](Cc1ccccc1)C(=O)N[C@@H]1C(=O)N2C(C(=O)O)=C(CSc3ccccc3)CSC12 ; O=C(Cc1cccs1)N[C@@H]1C(=O)N2C(C(=O)O)=C(CSc3ccccc3)CSC12] 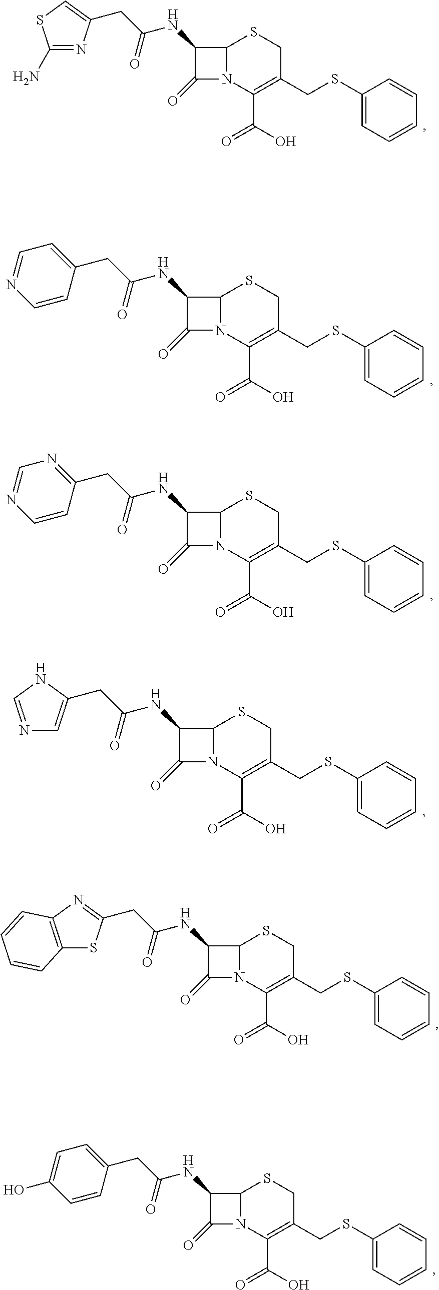 [SMILES: Nc1nc(CC(=O)N[C@@H]2C(=O)N3C(C(=O)O)=C(CSc4ccccc4)CSC23)cs1 ; O=C(Cc1ccncc1)N[C@@H]1C(=O)N2C(C(=O)O)=C(CSc3ccccc3)CSC12 ; O=C(Cc1ccncn1)N[C@@H]1C(=O)N2C(C(=O)O)=C(CSc3ccccc3)CSC12 ; O=C(Cc1cnc[nH]1)N[C@@H]1C(=O)N2C(C(=O)O)=C(CSc3ccccc3)CSC12 ; O=C(Cc1nc2ccccc2s1)N[C@@H]1C(=O)N2C(C(=O)O)=C(CSc3ccccc3)CSC12 ; O=C(Cc1ccc(O)cc1)N[C@@H]1C(=O)N2C(C(=O)O)=C(CSc3ccccc3)CSC12] 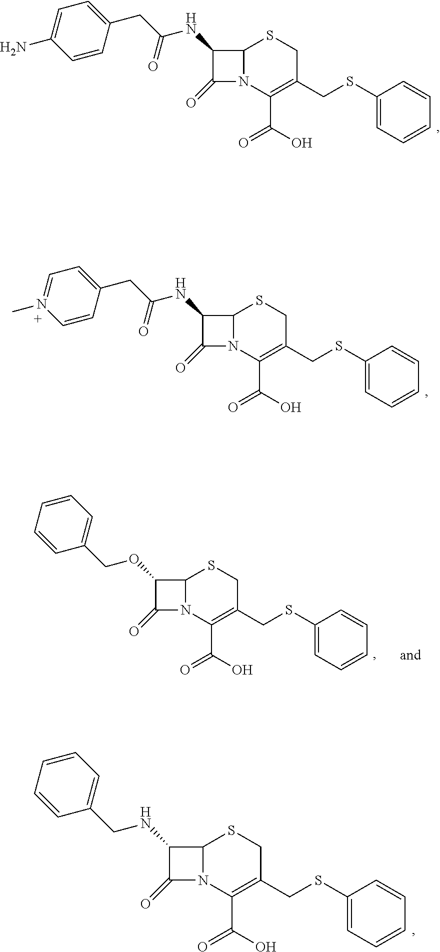
or a salt, stereoisomer, tautomer, polymorph, or solvate thereof.

7. The compound of claim 10, wherein the compound has the structure of:

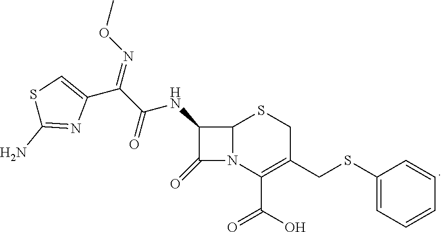 [SMILES: CO/N=C(/C(=O)N[C@@H]1C(=O)N2C(C(=O)O)=C(CSc3ccccc3)CSC12)c1csc(N)n1]

8. The compound of claim 1, wherein T³ is a benzenethiol containing group selected from the group consisting of:

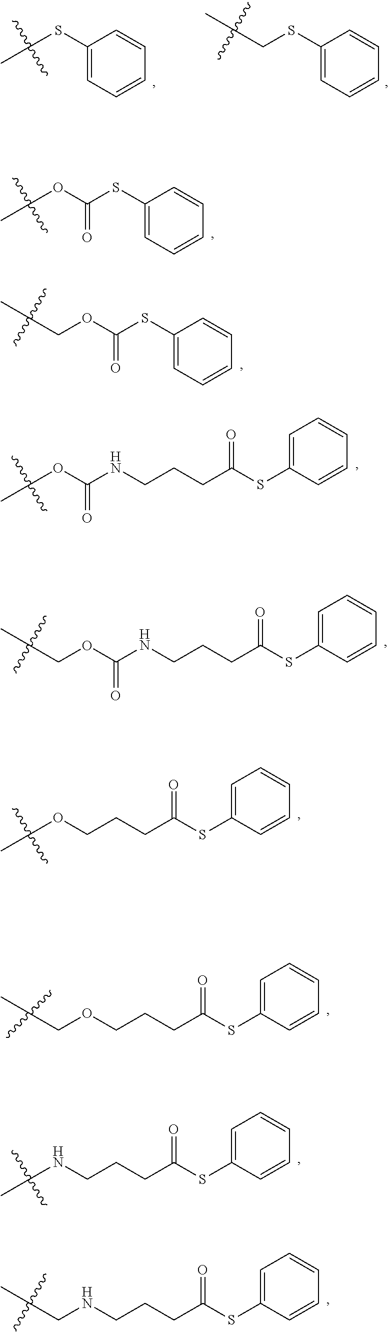

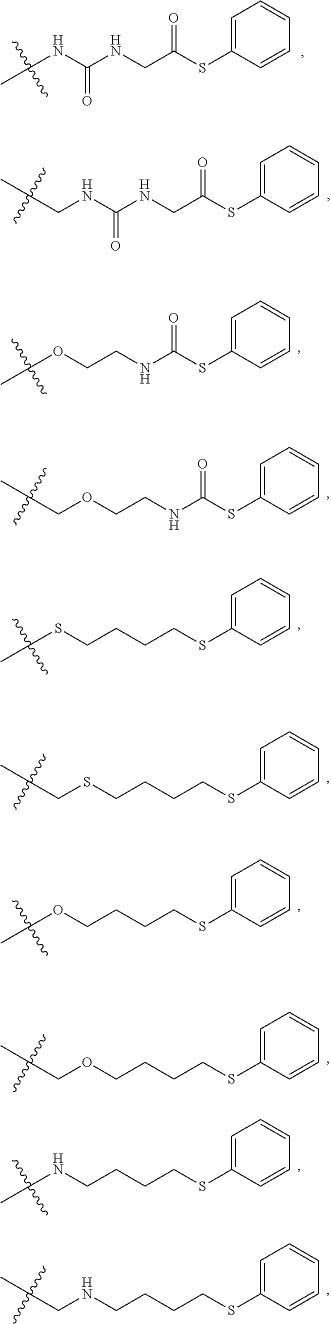

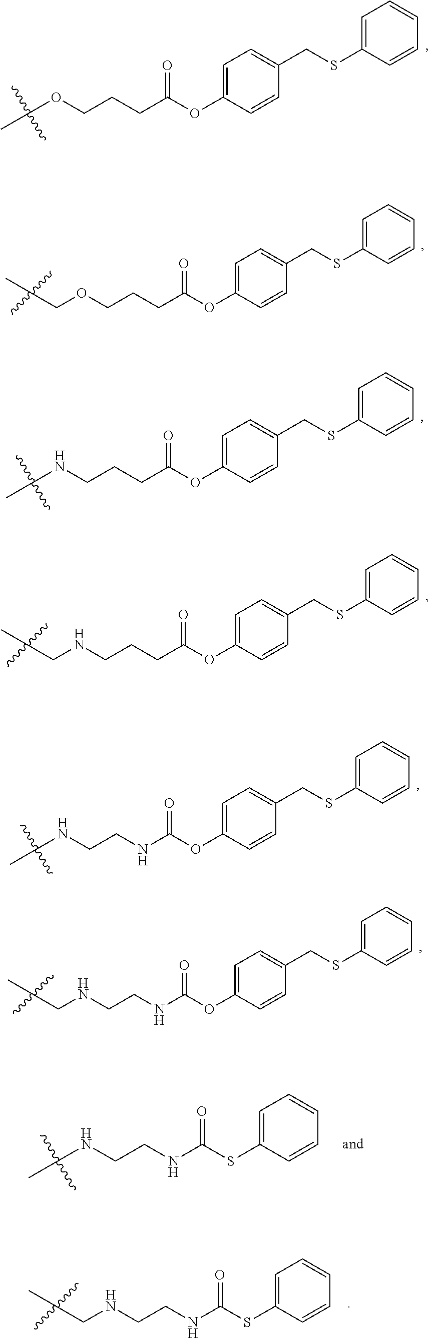

9. The compound of claim 1, wherein the compound has the structure of Formula II(a): 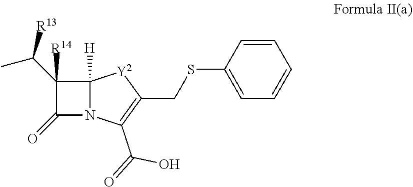
or a salt, stereoisomer, tautomer, polymorph, or solvate thereof, wherein:
Y² is 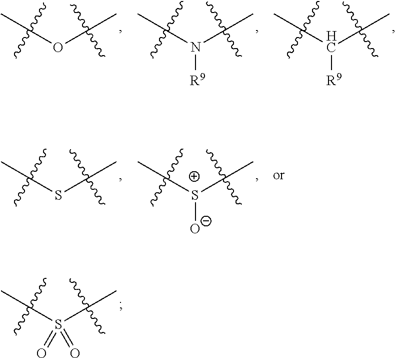
R⁹, R¹³ and R¹⁴ are independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, optionally substituted (C₁-C₆)alkyl, optionally substituted (C₁-C₆)alkenyl, optionally substituted (C₁-C₆)alkynyl, optionally substituted (C₅-C₇) cycloalkyl, optionally substituted aryl, optionally substituted benzyl, and optionally substituted heterocycle.

10. The compound of claim 1, wherein the compound has the structure of Formula II(b): 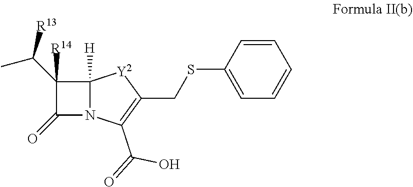
or a salt, stereoisomer, tautomer, polymorph, or solvate thereof, wherein:
Y² is 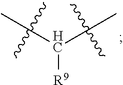
R⁹, R¹³ and R¹⁴ are independently selected from H, D, hydroxyl, nitrile, halo, amine, nitro, amide, thiol, aldehyde, carboxylic acid, alkoxy, optionally substituted (C₁-C₄) ester, optionally substituted (C₁-C₄) ketone, and optionally substituted (C₁-C₆)alkyl.

11. The compound of claim 1, wherein the compound has a structure selected from:

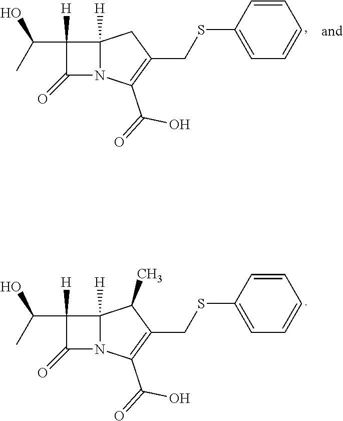 [SMILES: C[C@@H](O)[C@H]1C(=O)N2C(C(=O)O)=C(CSc3ccccc3)C[C@H]12 ; "," ; "and" ; C[C@@H](O)[C@H]1C(=O)N2C(C(=O)O)=C(CSc3ccccc3)[C@H](C)[C@H]12 ; "."]

12. The compound of claim 1, wherein the compound is substantially a single enantiomer or a single diastereomer, wherein the compound has an (R) stereocenter.

13. A method using a compound of claim 1, to detect the presence of one or more target β-lactamases in a sample, comprising:
(1) adding reagents to a sample suspected of comprising one or more target β-lactamases, wherein the reagents comprise:
(i) the compound of claim 1;
(ii) a chromogenic substrate for a cysteine protease;
(iii) a caged/inactive cysteine protease; and
(iv) optionally, an inhibitor to specific type(s) or class(es) of β-lactamases;
(2) measuring the absorbance of the sample;
(3) incubating the sample for at least 10 min and then re-measuring the absorbance of the sample;
(4) calculating a score by subtracting the absorbance of the sample measured in step (2) from the absorbance of the sample measured in step (3);
(5) comparing the score with an experimentally determined threshold value; wherein if the score exceeds a threshold value indicates that the sample comprises the one or more target β-lactamases; and wherein if the score is lower than the threshold value indicates the sample does not comprise the one or more target β-lactamases.

14. The method of claim 13, wherein:
for step (1), the sample is obtained from a subject, wherein the subject is a human patient that has or is suspected of having a bacterial infection, wherein the human patient has or is suspected of having a urinary tract infection;
for step (1), the sample is a blood sample, a urine sample, a cerebrospinal fluid sample, a saliva sample, a rectal sample, a urethral sample, or an ocular sample, wherein for step (1), the sample is a blood sample or urine sample, wherein the sample is a urine sample; or
for step (1), the one or more target β-lactamases are selected from penicillinases, extended-spectrum β-lactamases (ESBLs), inhibitor-resistant β-lactamases, AmpC-type β-lactamases, and carbapenemases, wherein the ESBLs are selected from TEM β-lactamases, SHV β-lactamases, CTX-M β-lactamases, OXA β-lactamases, PER β-lactamases, VEB β-lactamases, GES β-lactamases, and IBC β-lactamase, where the one or more target β-lactamases comprise CTX-M β-lactamases, wherein the carbapenemases are selected from metallo-β-lactamases, KPC β-lactamases, Verona integron-encoded metallo-β-lactamases, oxacillinases, CMY β-lactamases, New Delhi metallo-β-lactamases, Serratia marcescens enzymes, IMIpenem-hydrolysing β-lactamases, NMC β-lactamases and CcrA β-lactamases, wherein the one or more target β-lactamases comprise CMY β-lactamases and/or KPC β-lactamases, wherein the one or more target β-lactamases further comprise CTX-M β-lactamases.

15. The method of claim 13, wherein for step (1)(ii), the chromogenic substrate for a cysteine protease is a chromogenic substrate for papain, bromelain, cathepsin K, calpain, caspase-1, galactosidase, seperase, adenain, pyroglutamyl-peptidase I, sortase A, hepatitis C virus peptidase, sindbis virus-type nsP2 peptidase, dipeptidyl-peptidase VI, deSI-1 peptidase, TEV protease, amidophosphoribosyl transferase precursor, gamma-glutamyl hydrolase, hedgehog protein, or dmpA aminopeptidase, wherein the chromogenic substrate for a cysteine protease is a chromogenic substrate for papain, wherein the chromogenic substrate for papain is selected from the group consisting of azocasein, L-pyroglutamyl-L-phenylalanyl-L-leucine-p-nitroanilide (PFLNA), Nα-benzoyl-L-arginine 4-nitroanilide hydrochloride (BAPA), pyroglutamyl-L-phenylalanyl-L-leucine-p-nitroanilide (Pyr-Phe-Leu-pNA), and Z-Phe-Arg-β-nitroanilide, wherein the chromogenic substrate for papain is BAPA.

16. The method of claim 13, wherein for step (1)(iii), the caged/inactive cysteine protease comprises a cysteine protease selected from the group consisting of papain, bromelain, cathepsin K, calpain, caspase-1, galactosidase, seperase, adenain, pyroglutamyl-peptidase I, sortase A, hepatitis C virus peptidase, sindbis virus-type nsP2 peptidase, dipeptidyl-peptidase VI, deSI-1 peptidase, TEV protease, amidophosphoribosyl transferase precursor, gamma-glutamyl hydrolase, hedgehog protein, and dmpA aminopeptidase, wherein the caged/inactive cysteine protease comprises papain, wherein the caged/inactive cysteine protease is papapin-S—SCH₃.

17. The method of claim 13, wherein for step (1)(iii), the caged/inactive cysteine protease can be re-activated by reaction with low molecular weight thiolate anions or inorganic sulfides, wherein the caged/inactive cysteine protease can be reactivated by reaction with a benzenethiolate anion, wherein the one or more target β-lactamases react with the compound of (i) to produce a benzenethiolate anion, wherein the benzenethiolate anion liberated from the compound of step (I1)(i) reacts with the caged/inactive cysteine protease to reactivate the cysteine protease, wherein the caged/inactive cysteine protease is papain-S—SCH₃, wherein the chromogenic substrate for a cysteine protease is BAPA.

18. The method of claim 13, wherein for step (2), the absorbance of the sample is measured at 0 min, wherein for step (3), the sample is incubated for 15 min to 60 min, wherein the sample is incubated for 30 min.

19. The method of claim 13, wherein for steps (2) and (3), the absorbance of the sample is measured at a wavelength of 400 nm to 450 nm, wherein for steps (2) and (3), the absorbance of the sample is measured at a wavelength of 405 nm.

20. The method of claim 13, wherein for steps (2) and (3), the absorbance of the sample is measured using a spectrophotometer, or a plate reader, wherein for step (5), the experimentally determined threshold value was determined by analysis of a receiver operating characteristic (ROC) curve generated from an isolate panel of bacteria that produce β-lactamases, wherein the one of more target β-lactamases have the lowest limit of detection (LOD) in the isolate panel, wherein the method is performed with and without the inhibitor to specific type(s) or class(es) of β-lactamase in step (1)(iv), wherein a measured change in the score of step (4), between the method performed without the inhibitor and the method performed with the inhibitor indicates that the specific type or class of β-lactamases is present in the sample, wherein the inhibitor to specific type(s) or class(es) of β-lactamases is an inhibitor to class of β-lactamases selected from the group consisting of penicillinases, extended-spectrum β-lactamases (ESBLs), inhibitor-resistant β-lactamases, AmpC-type β-lactamases, and carbapenemases, wherein the inhibitor to a specific type(s) or class(es) of β-lactamases inhibits ESBLs but does not inhibit AmpC-type β-lactamases, wherein the inhibitor is clavulanic acid or sulbactam.


SEQUENCE LISTING (20 sequences)

SEQ ID NO: 1 - DNA, 39 bp, Artificial Sequence
  OXA-1 forward primer
tatacatatgtcaacagatatctctactgttgcatctcc

SEQ ID NO: 2 - DNA, 47 bp, Artificial Sequence
  OXA-1 reverse primer
ggtgctcgagtaaatttagtgtgtttagaatggtgatcgcatttttc

SEQ ID NO: 3 - DNA, 26 bp, Artificial Sequence
  SHV-12 forward primer
tatacatatgagcccgcagccgcttg

SEQ ID NO: 4 - DNA, 31 bp, Artificial Sequence
  SHV-12 reverse primer
ggtgctcgaggcgttgccagtgctcgatcag

SEQ ID NO: 5 - DNA, 32 bp, Artificial Sequence
  TEM-20 forward primer
tatacatatgcacccagaaacgctggtgaaag

SEQ ID NO: 6 - DNA, 34 bp, Artificial Sequence
  TEM-20 reverse primer
ggtgctcgagccaatgcttaatcagtgaggcacc

SEQ ID NO: 7 - DNA, 21 bp, Artificial Sequence
  TEM-268 forward primer
ggtcgccgcatacactattct

SEQ ID NO: 8 - DNA, 22 bp, Artificial Sequence
  TEM-268 reverse primer
tcctccgatcgttgtcagaagt

SEQ ID NO: 9 - DNA, 20 bp, Artificial Sequence
  SHV-68 forward primer
cgcagccgcttgagcaaatt

SEQ ID NO: 10 - DNA, 20 bp, Artificial Sequence
  SHV-68 reverse primer
ctgttcgtcaccggcatcca

SEQ ID NO: 11 - DNA, 19 bp, Artificial Sequence
  CTX1-681 forward primer
actgcctgcttcctgggtt

SEQ ID NO: 12 - DNA, 21 bp, Artificial Sequence
  CTX1-681 reverse primer
tttagccgccgacgctaatac

SEQ ID NO: 13 - DNA, 20 bp, Artificial Sequence
  CTX9-681 forward primer
cttaccgacgtcgtggactg

SEQ ID NO: 14 - DNA, 20 bp, Artificial Sequence
  CTX9-681 reverse primer
cgatgattctcgccgctgaa

SEQ ID NO: 15 - DNA, 20 bp, Artificial Sequence
  CMY-877 forward primer
tgggagatgctgaactggcc

SEQ ID NO: 16 - DNA, 21 bp, Artificial Sequence
  CMY-877 reverse primer
atgcacccatgaggctttcac

SEQ ID NO: 17 - DNA, 21 bp, Artificial Sequence
  KPC-625 forward primer
tggctaaagggaaacacgacc

SEQ ID NO: 18 - DNA, 22 bp, Artificial Sequence
  KPC-625 reverse primer
gtagacggccaacacaataggt

SEQ ID NO: 19 - DNA, 23 bp, Artificial Sequence
  rpoB forward primer
aaggcgaatccagcttgttcagc

SEQ ID NO: 20 - DNA, 25 bp, Artificial Sequence
  rpoB reverse primer
tgacgttgcatgttcgcacccatca